(19) United States
(12) Patent Application Publication
(10) Pub. No.: US 2006/0189550 A1
(43) Pub. Date: Aug. 24, 2006

(54) Immunostimulatory, Covalently lipidated oligonucleotides

(75) Inventors: Zi-Hua Jiang, Thunder Bay (CA); Rao Koganty, Edmonton (CA); Damayanthi Yalamati, Edmonton (CA); Myung-Gi Baek, Edmonton (CA)

Correspondence Address: BROWDY AND NEIMARK, P.L.L.C.;624 NINTH STREET, NW, SUITE 300, WASHINGTON, DC, 20001-5303, US

(73) Assignee: Biomira Inc., Edmonton (CA)

(21) Appl. No.: 10/502,085

(22) PCT Filed: Feb. 4, 2003
(86) PCT No.: PCT/CA03/00135

§ 371 Date: Apr. 12, 2005

Related U.S. Application Data
(60) Provisional application No. 60/353,195, filed on Feb. 4, 2002.

(51) Int. Cl. A61K 48/00; C07J 17/00; C07H 21/04

(52) U.S. Cl. 514/26; 514/44; 536/6.1; 536/23.1

(57) ABSTRACT

Covalently lipidated oligonucleotides comprising the CpG dinucleotide unit, or an analogue thereof, may be used as immunostimulatory agents to protect against a disease caused by a cancer cell or a pathogen, either alone or in conjunction with immunogens and/or non-immunological agents. Lipidated oligonucleotides with special backbones, lipidated oligonucleotides with fewer than eight nucleotides, and lipidated oligonucleotides comprising a plurality of CpG dinucleotide-containing segments connected by a long internucleoside linkage are of particular interest. These compounds are also novel per se.

This application is a nonprovisional of U.S. provisional appl. 60/353,195 filed Feb. 4, 2002, which is hereby incorporated by reference in its entirety.

### BACKGROUND OF THE INVENTION

1. Field of the Invention

This invention relates to the use of covalently lipidated oligonucleotides comprising the CpG dinucleotide unit, or an analogue thereof, as immunostimulatory agents. Lipidated oligonucleotides with special backbones, lipidated oligonucleotides with fewer than eight nucleotides, and lipidated oligonucleotides comprising a plurality of CpG dinucleotide-containing segments connected by a long internucleoside linkage are of particular interest.

2. Description of the Background Art

Effective host defense against invading microorganisms requires the detection of foreign pathogens and the rapid deployment of an antimicrobial effector response (Krutzik et al, 2001). Indeed, the innate immune system detects the presence and the nature of the infection by recognizing the constitutive and conserved products of microbial metabolism, which can be viewed as molecular signatures of microbial invaders (Janeway, 1992), and also called pathogen-associated molecular patterns (PAMPs). PAMPs are recognized by various pattern-recognition receptors (PRRs) of the innate immune system, which are expressed on the cell surface, in intracellular compartments, or secreted into the blood stream and tissue fluids. Also, the innate immune system provides the first line of host defense, and controls the initiation and determination of the effector class of the adaptive immune response (Medzhitov, 2001).

Toll-Like Receptor Ligands

The recent discovery and characterization of the Toll-like receptor (TLR) family have incited new interest in the field of innate immunity. TLRs are also pattern-recognition receptors (PRRs) that have a unique and essential function in animal immunity. In mammalian species there are at least ten TLRs currently known (Medzhitov, 2001), and each seems to have a distinct function in innate immune recognition. Dozens of TLR ligands have been identified (Akira et al, 2001).

Though quite diverse in structure and origin, TLR ligands have several common features. For instance, most TLR ligands are conserved microbial products (PAMPs) that signal the presence of infection; and many individual TLRs can recognize several structurally unrelated ligands. It is obvious that TLR ligands are potent activators of innate immune system, which in turn directs and determines the adaptive immune response.

CpG Motifs

Probably the most enigmatic example of pattern recognition is the recognition of unmethylated CpG motifs in bacterial DNA by TLR9 (Hemmi et al, 2000). As a matter of fact, unmethylated bacterial DNA in a particular sequence context (the so-called CpG motif) has been known for its potent immune stimulatory activity for quite some time (Krieg et al, Nature, 1995).

WO98/18810 (University of Iowa Research Foundation) teaches that certain nucleic acids containing unmethylated CpG dinucleotides activate lymphocytes in a subject and redirect a subject's immune responses from a Th2 to a TH1, i.e., increase production of Th1 cytokines including IL-12, IFN-gamma and GM-CSF. In particular, it discloses an isolated immunostimulatory nucleic acid sequence of about 8-30 bases “represented” by the formula
5′N₁X₁CGX₂N₂3′
where at least one nucleotide separates consecutive CpGs; X₁ is A, G or T, X₂ is C or T, N is any nucleotide, and N1+N2 is 0-26 nucleotides, with the proviso that the latter does not contain a CCGG quadmer or more than one CCG or CGG trimer. With respect to stimulation of murine cells, a preference is expressed for a CpG flanked by two 5′ purines (preferably GpA) and two 3′ pyrimidines (preferably TpT or TpC).

The authors reported that oligomers shorter than 8 bases were non-stimulatory (page 25, lines 16-17); the tested oligomer was a 7-mer (Table 1, 4e). See also Sonehara, et al., “Hexamer Palindromic Oligonucleotides with 5′-CG-3′ Motif(s) Induce Production of Interferon,” J. Interferon & Cytokine Res., 16:799-803 (1996)(IFN-inducing activity of ACGT “insignificant”). In contrast, in the present invention, we have found that if lipidated, even a dinucleotide by itself has activity.

WO98/40100 (Ottawa Civic Loeb Research Laboratory, Qi-Agen GmbH, and University of Iowa Research Foundation), WO99/51259 (University of Iowa Research Foundation), WO99/61056 (Loeb Health Research Institute at the Ottawa Hospital, CPG Immunopharmaceuticals, Inc.) have similar teachings. While WO98/40100 defines an oligonucleotide as being at least five bases in length, it nonetheless teaches that for the desired immunostimulatory activity, at least 8 bases are needed. WO01/97843 says that the immunostimulatory nucleic acid can have any length greater than 6 nucleotides (p. 7, l. 12). WO99/61056 (p3, L30) implies that a hexanucleotide can induce mucosal immunity, although an octanucleotide is preferred (p 8, l. 17).

WO01/12804 (Hybridon, Inc.) teaches that it is desirable that the two bases immediately flanking the CpG on its 5′ end be 2′-OMe substituted.

WO01/97843 (University of Iowa Research Foundation) teaches that it is desirable that the CpG oligonucleotide be “T-rich”, i.e., greater than 25% T, more preferably, greater than 40%, 50%, 60%, 80% or 90% T. It teaches that it is desirable that it comprise at least one poly-T motif consisting of at least three consecutive T bases. It expresses a preference for longer poly-T motifs (at least 4-9 Ts) and for a plurality of poly-T motifs (at least 2-8). Likewise, it discloses the desirability of poly-A, poly-C, and poly-G motifs.

WO00/54803 (Panacea Pharmaceuticals, LLC) relates to use of the CpG-containing oligonucleototides to ameliorate allergic responses to immunogens. See also DeKruyff, U.S. Pat. No. 6,086,898 (2000), on converting a Th2-type allergic immune response into a Th1-type immune response.

WO01/07055 teaches use of a CpG-oligonucleotide having a particular topology, for higher stability in vivo.

Lipoteichoic Acid; Doubse-Stranded Ribonucleic Acid

Structurally related lipo-teichoic acid (LTA) from Gram-positive bacteria and double-stranded RNA (dsRNA) from viruses are also well known for their properties of activating host innate immunity. Recent studies have shown that TLR4 is involved in the recognition of LTA (Takeuchi et al, 1999) and TLR3 functions as a cell-surface receptor for dsRNA (Alexopoulou et al, 2001).

Teichoic acids are polyol phosphate polymers, with either ribitol or glycerol linked by phosphodiester bonds. Substituent groups on the polyol chains of the naturally occurring teichoic acids can include D-alanine (ester-linked), N-acetylglucosamine, and glucose. In the ribitol teichoic acids, there are 1,5-phosphodiester linkages. In the glycerol teichoic acids, there are 1,2- or 1,3-phosphodiester linkages. The glycerol teichoic acids may be unsubstituted, or substituted (e.g. alanyl or glycosyl) at the remaining position.

Glycerol Nucleic Acids

Usman, Juby and Ogilvie, “Preparation of Glyceronucleoside Phosphoramidite Synthons and Their Use in the Sold Phase Synthesis of Acyclic Oligonucleotides” Tetrahedron Lett., 29: 4831-4 (1988) descibes the synthesis of homooligomers (2-8 units long) of the nucleotides in which either adenine or thymine are part of a glyceronucleoside. It is noted in passing that glyceronucleosides per se, especially those containing purines (adenine and thymine) as the base component, are potent antiviral agents.

Schneider and Benner, “Oligonucleotides Containing Flexible Nucleoside Analogues,” J. Am. Chem. Soc. 112: 453-55 (1990) disclose oligonucleotides in which ribose is replaced by a glycerol derivative. However, they found that the change in backbone reduced the ability of the oligonucleotide to form a stable duplex structure. Each glycerol nucleoside reduced the melting point of the duplex DNA by about 9-15° C. The oligonucleotides synthesized by Schneider and Benner were at least 9 bases long, and none of them comprised 5′-CG-3′.

Other Oligonucleotide Analogues

Oligonucleotide analogues, especially those with modified internucleoside linkages, are known in the art. See e.g. Cook, U.S. Pat. No. 5,717,083; Weis, U.S. Pat. No. 5,677,439; Rosch, U.S. Pat. No. 5,750,669; Cook, U.S. Pat. No. 6,114,513; Cook, U.S. Pat. No. 6,111,085; Uhlmann, et al., “PNA: Synthetic Polyamide Nucleic Acids with Unusual Binding Properties,” Angew. Chem. Int. Ed. 37:2796-2823 (1998).

Chemically Modified Nucleic Acids

Englisch and Gauss, “Chemically Modified Oligonucleotides as Probes and Inhibitors”, Angew. Chem. Int. Ed. Engl., 30: 613-29 (1991) make reference to oligonucleotides modified to include psoralen, acridine, biotin, or enzymes.

Known terminal radicals (hydroxyl substituents) include 1-ethoxyethyl, ethoxymethyl, benzhydryl, benzyl, trityl, monomethoxytrityl, dimethoxytrityl, methyl, ethyl, acetyl, tosyl, tetrahydropyranyl, trifluoroacetyl, aminoacyl, glycyl, leucyl, cyanoethyl, anisyl, benzyl, and phenyl, and, as bifunctional protecting groups (usually bridging 2′ and 31),

Known terminal glycol-protecting (bifunctional) radicals, bridging the 2′ and 3′ hydroxyls unless otherwise indicated, include isopropylidene, borate, and carbonyl 2′:3′-phosphate (cyclic).

N-protecting radicals used in synthetic work include benzoyl, benzyl, tosyl, trityl, anisoyl, benzhydryl (diphenylmethyl), monomethoxytrityl (p-anisyldiphenylmethyl), dimethoxytrityl (di-p-anisylphenylmethyl), tetrahydropyranyl, dansyl, and N-cyclohexyl-N′[-(4-methylmorpholino)amidino].

Phosphoric acid protecting groups include 5′-cyanoethyl; 3′(or 2′)-cyanoethyl, anisyl (MeOPh), benzyl, and phenyl.

Lipidated Nucleic Acids

Hostetler, U.S. Pat. No. 5,827,831 teaches that oral delivery of many clases of drugs is facilitated by converting drugs having suitable functional groups to 1-O-alkyl, 1-O-acyl, 1-S-acyl, and 1-S-alkyl-sn-glycero-3-phosphate derivatives; he refers to these derivatives as lipid prodrugs. The classes of drugs taught by Hostetler include “anticancer agents, comprising nucleoside analogues, for example, 9-beta-D-arabinofuranosylcytosine (hereinafter, cytosine arabinoside or ara-C), 9-beta-D-arabinofuranosyladenine (hereinafter, adenine arabinoside or ara-A), 5-fluorouridine, 6-mercaptopurine riboside, or 2′-ara-fluoro-2-chlorodeoxyadenosine”, and “antiviral nucleosides, particularly the 1-O-alkyl phospholipid derivatives of those antiviral nucleosides disclosed in U.S. Pat. No. 5,223,263”. In the antiviral category, specific reference is made to 3′deoxy, 3′-azidothymidine (AZT), acyclovir, foscarnet, ganciclovir, idoxuridine, ribavarin, 5-fluoro-3′-thia-2′,3′-dideoxycytidine, trifluridine, and vidarabine. There is no reference to lipidation of any oligonucleotides.

Sridhar, U.S. Pat. No. 5,756,352 discloses thiocationic lipid-nucleic acid conjugates. The cationic lipid binds noncovalently to the anionic nucleic acid molecules. The present invention requires covalent attachment of a lipophilic group to the oligonucleotide.

Cheng, et al. U.S. Pat. No. 5,646,126 discloses double stranded oligonucleotides having a lipophilic group, preferably a steroid structure, attached to the 3′ end. The oligonucleotides comprised 8-18 bases (per strand). They do not disclose or suggest any shorter oligonucleotides, or any molecules without at least some double-stranded structure.

Cheng et al. conceived of three types of molecules with double stranded structure. In type 1, the oligonucleotide is palindromic, so two molecules together form a duplex. In type 2, there are two different but substantially complementary strands, which hybridize to form the duplex. Finally, in type 3, the oligonucleotides are at least partially self-complementary, so they “hairpin” to form a double-stranded structure.

The oligonucleotides of interest to them were those with anticancer activity. They do not disclose or suggest that any of his oligonucleotides have immunostimulatory activity.

Several of these oligonucleotides (e.g., 120H, 128H, 001H, 167H, 002H, 089H, 589H, 178H, 678H) comprise 5′-CG-3′. The 3′ modifications of sequence 128H (CACACGTGTG)(SEQ ID NO: 1) included cholesterol*, hexylamine, acridine, hexanol, hexadecane, cholestanol*, ergosterol, stigmastanol*, stigmasterol*, and methyl-lithacholic acid; only the starred modifications had anticancer activity (see Cheng FIG. 10). Thus, Cheng et al. discouraged further experimentation with 3′ lipophilic modifications other than those with the steroid skeleton of his Formula 1. Cheng et al. do not disclose or suggest any 5′ lipophilic attachments.

While Cheng et al. contemplated the possibility of backbone modification, especially, phosphorothioate (P—S0 linkages, they did not specifically suggest peptide-nucleic acid (PNA) or glycerol nucleic acid (GNA) backbones. Indeed, since GNA backbones reduce duplex stability, use of a GNA backbone would have been contrary to Cheng et al.'s teaching of oligonucleotide duplexes.

Targeted Nucleic Acids

Manoharan, U.S. Pat. No. 6,300,319 discloses attaching a cell surface receptor ligand to an oligonucleotide to facilitate delivery of the oligonucleotide to the cell in question. Manoharan notes that natural oligonucleotides are polyanionic and poorly penetrate cells, while the methylphosphonates are neutral and are taken up much more readily. The ligands contemplated by Manoharan are primarily carbohydrates (targeting cell surface lectins) sch as galactose, N-acetylgalactosamine, fucose, mannose, and sialic acid. These would not be considered lipophilic groups (see Table K-2, below).

### SUMMARY OF THE INVENTION

The present invention relates to a method of stimulating the immune system which comprises the administration of an oligonucleotide which comprises (1) one or more “CxG” dinucleotide units (i.e., a nucleotide presenting the nucleobase cytosine is linked directly to a nucleotide presenting the nucleobase guanine), or an immunostimulatory analog thereof, and (2) one or more covalently attached (incorporated) lipophilic groups.

The presence of the CxG dinucleotide unit renders the oligonucleotide a potential TLR ligand.

The lipophilic group can facilitate the penetration of the oligonucleotide into a cell membrane, whether directly or through incorporation of the lipidated oligonucleotide into a liposomal drug delivery formulation. It may also interact with cell surface lipid receptors.

We have discovered that even CxG-containing oligonucleotides of less than five nucleobases have immunostimulatory activity, and we credit this to the presence of the lipophilic group.

We have also discovered that the oligonucleotide may comprise an alternative backbone, such as a glycerol-nucleic acid backbone, and have immunostimulatory activity.

The oligonucleotides of the present invention may, but need not, have the cytotoxicity for cancer cells sought by Cheng, and they may be administered to persons in need of immune system stimulation who are not suffering from cancer, as well as to those who are.

The present invention also relates to certain classes of lipidated oligonucleotides, as compounds per se. (The term “lipidated oligonucleotide” simply means an oligonucleotide having one or more lipophilic groups, as hereafter defined.)

In one class, the number of nucleotides in the lipidated oligonucleotides is less than eight (the smallest lipidated oligonucleotide taught by Cheng, U.S. Pat. No. 5,646,126). Eight is also the minimum number of nucleotides taught by WO98/18810 in connection with unlipidated CpG-containing oligonucleotides taught as immunostimulatory agents. More preferably it is less than five nucleotides. Compounds 1-7 in FIG. 7 are non-limiting examples of this class; compounds 1, 2, 6 and 7 are dinucleotides while compound 3, 4 and 5 are hexanucleotides.

In a second class, the lipidated oligonucleotides comprise a plurality of oligonucleotide segments, each of at least two of these segments containing at least one “CxG” dinucleotide unit, and at least two such “CxG”-containing segments being connected, directly or indirectly, by a moiety which comprises a “long” internucleoside linkage. A non-limiting example of such a multisegment lipidated oligonucleotide, with two CpG-containing segments connected by a long internucleoside linkage, appears in FIG. 3.

In a third class, the lipidated oligonucleotides comprise, not only at least one “CxG” dinucleotide unit, but also at least one pair of adjacent thymine nucleobases that have dimerized together as shown in FIG. 19.

### BRIEF DESCRIPTION OF THE DRAWINGS

FIG. 1: Structure-activity relationship of unmethylated CpG-containing nucleotide sequence

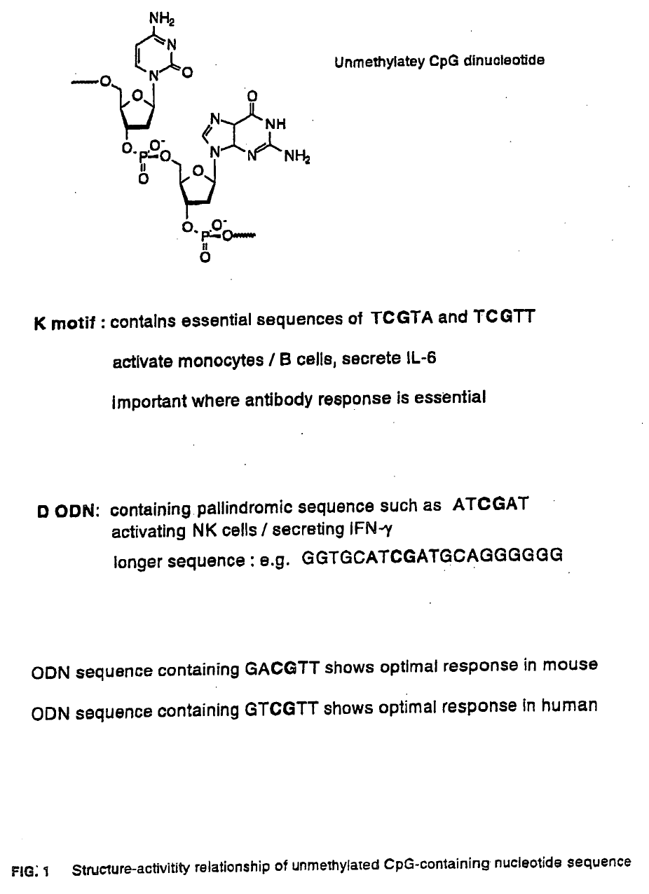

FIG. 2: CpG dinucleotide modified at 3′-end with various lipophilic groups.

FIG. 3: Modified CpG dinucleotide as divalent ligand; two segments, each presenting a CpG.

FIG. 4: Hexanucleotide ATCGAT modified at 5′-end with a lipophilic group.

FIG. 5: Comparison of structures of GNA (glycerol nucleic acid), DNA, and PNA (peptide nucleic acid).

FIG. 6: Hexanucleotide GtcgTT modified at 3′-end with a lipophilic group, wherein cg dinucleotide has glycerol-based nackbone.

FIG. 7: CpG-containing lipidated oligonucleotides with DNA (compounds 1-5), GNA (6) and PNA (7) backbones.

FIG. 8: Building blocks for solid-phase nucleotide synthesis by phosphoramidite method.

FIG. 9: Modification of long-chain amino alkyl controlled pore glass (lcaa-CPG) resin for the synthesis of lipidated oligonucleotides.

FIG. 10: Preparation of lipidated CpG dinucleotide 1 on solid phase.

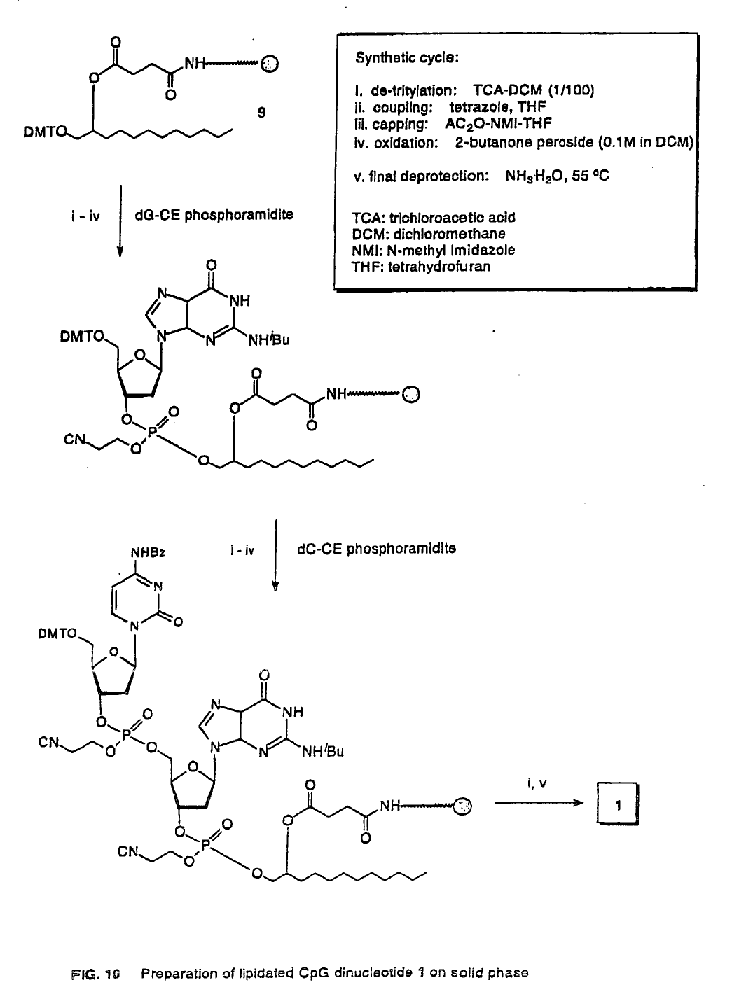

FIG. 11: Preparation of pentaerythritol-derived dilipo-alcohol 11 and its application for the synthesis of lipidated oligonucleotide 16.

FIG. 12: Preparation of glycerol-cytosine phosphoramidite 25.

FIG. 13: Preparation of glycerol-guanosine building block 32.

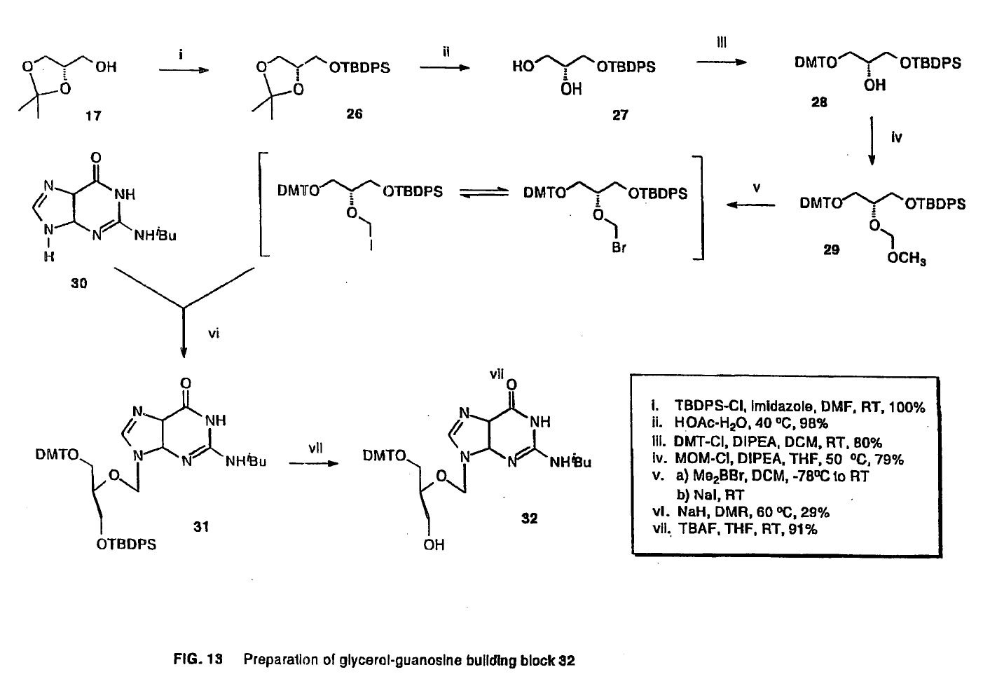

FIG. 14: Synthesis of glycerol-base CpG dinucleotide 6.

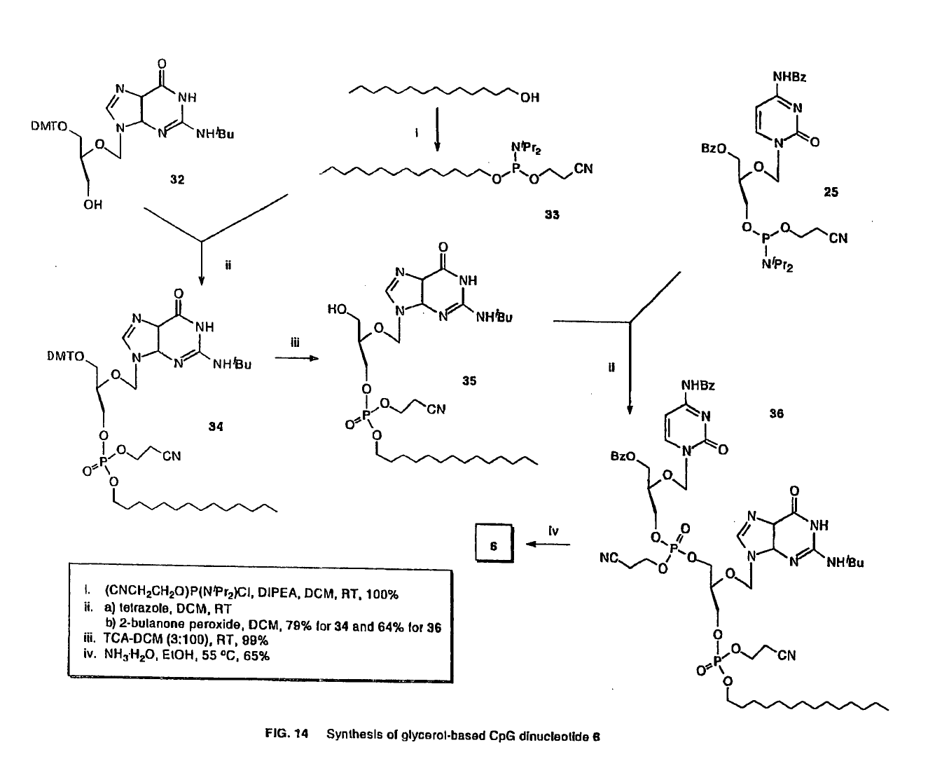

FIG. 15: Preparation of PNA-based CpG analogue 7 by standard solid phase peptide synthesis using Fmoc/Bhoc chemistry.

FIG. 16: Immunostimulatory adjuvant properties of CpG analogues 1-6. In vitro antigen specific proliferation of T cells from C57Bl/6 mice immunized with a single dose of BLP25 liposomal vaccine formulation. The vaccine dose contains 20 μg of MUC1-derived 25-mer lipopeptide as an antigen and 10 μg of one of synthetic CpG analogues 1-6 as an adjuvant. The adjuvant R595 lipid A (detoxified lipid A product isolated from Salmonella minnesota R595) is used for comparison.

FIG. 17: The first sequence is SEQ ID NO:11. The second structure is the structure of lipopeptide BP1-148, comprising a modified 25 amino acid sequence (SEQ ID NO:2) derived from tumor-associated MUC1 mucin. SEQ ID NO:2 corresponds to AAs 1-25 of SEQ ID NO:11, and to AAs 14-20, followed by AAs 1-18, of the MUC1 tandem repeat as set forth in SEQ ID NO:10.

The aforementioned BLP25 liposomal vaccine formulation is this lipopeptide in combination with one of the aforementioned adjuvants.

FIG. 18: A branched, three segment oligonucleotide with a long multivalent internucleoside linkage comprising a pentaerythritol element. The oligonucleotide further comprises a cholesterol residue as a lipophilic group.

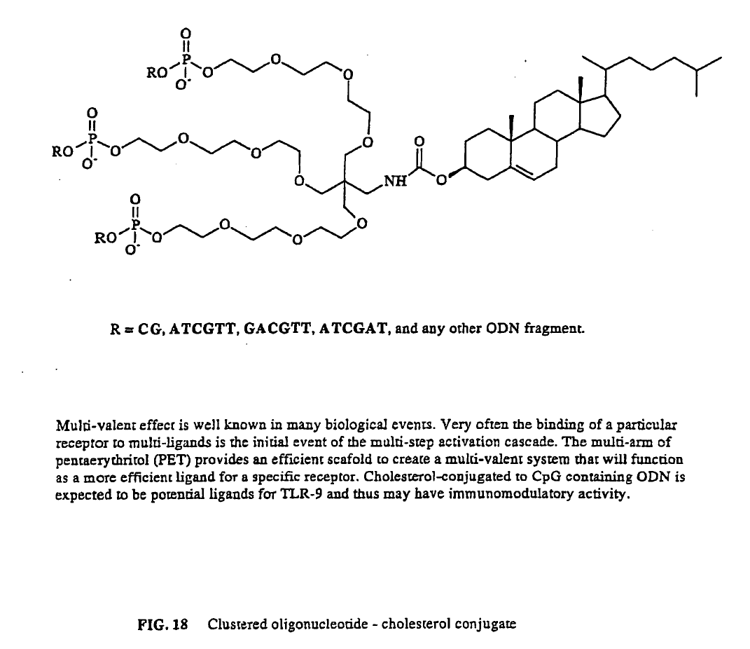

FIG. 19: CpG-containing oligonucleotides, with flanking bases, lipidated at 3′ end. Last oligonucleotide is SEQ ID NO:3.

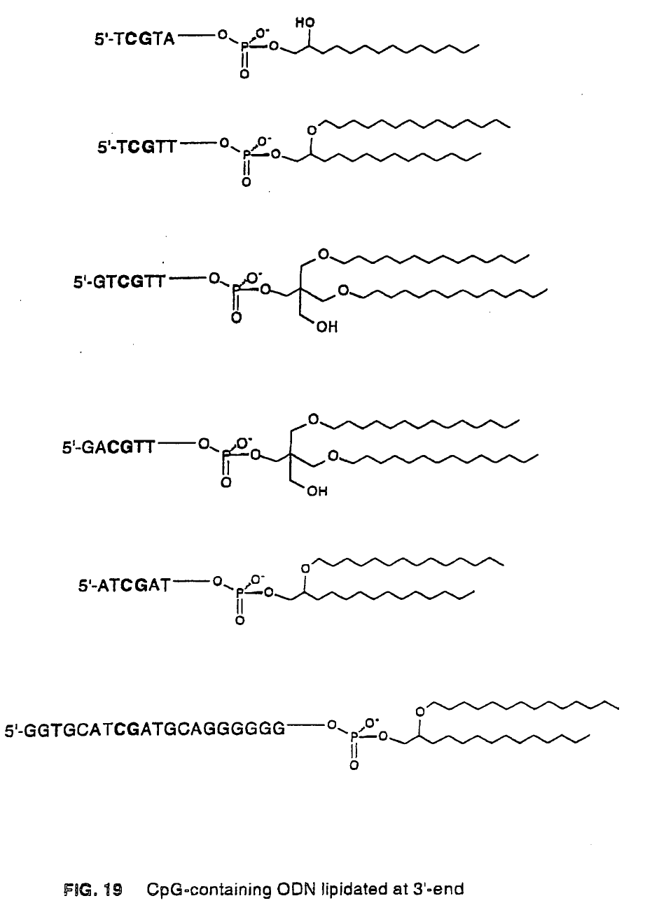

FIG. 20: lipidated oligonucleotide cyclized by long internucleoside linkage comprising lipophilic groups.

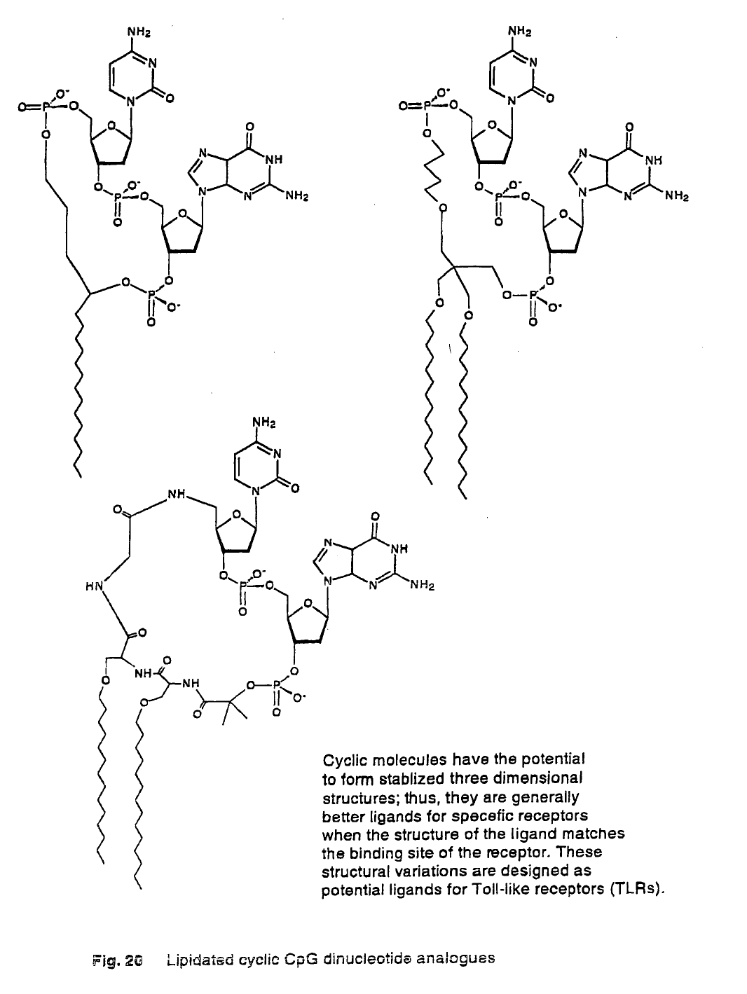

FIG. 21: Lipidated oligonucleotide comprising thymidine dimer as well as CpG dinucleotide.

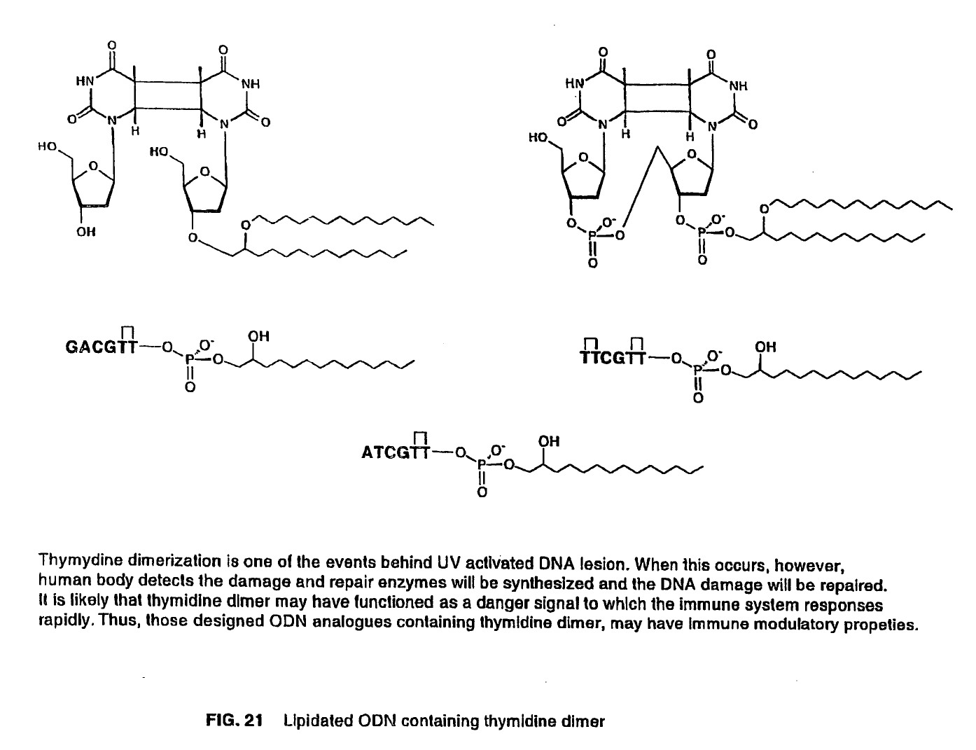

FIG. 22: Lipidated oligonucleotides with GNA backbone.

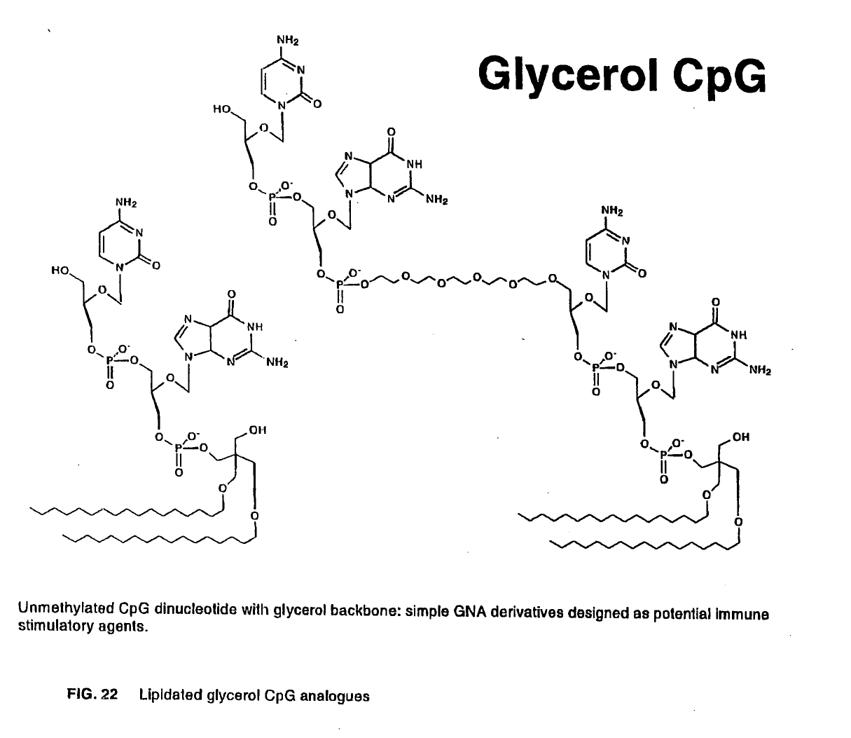

FIG. 23: Structures derived from lipoteichoic acid.

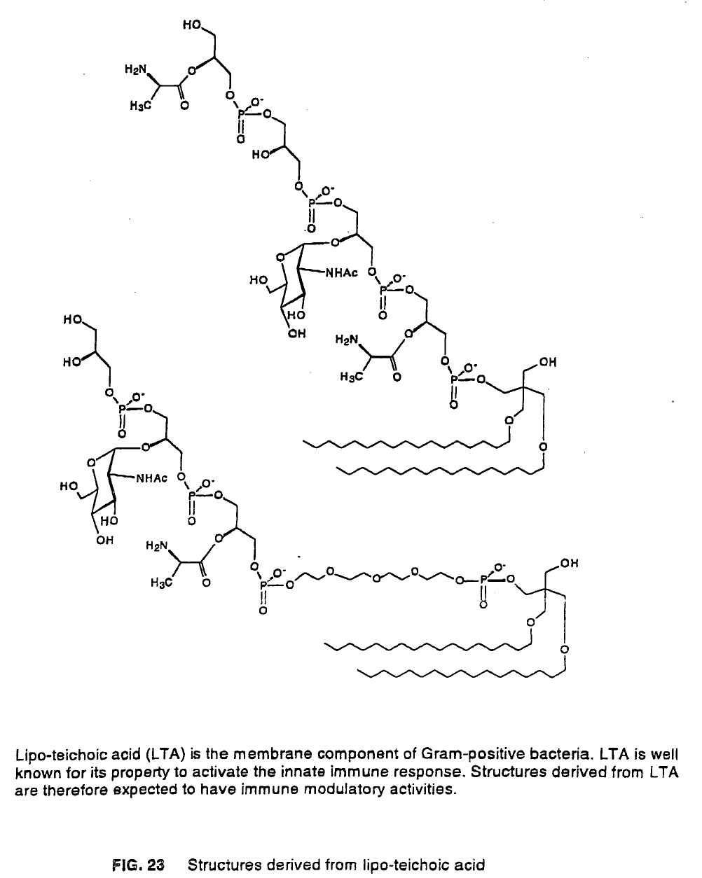

FIG. 24: Modified lipoteichoic acid backbone, wherein the ester linkage between the D-alanine and the secondary hydroxyl group of glycerol unit is replaced by an amide bond. This is expected to be more resistant to hydrolysis.

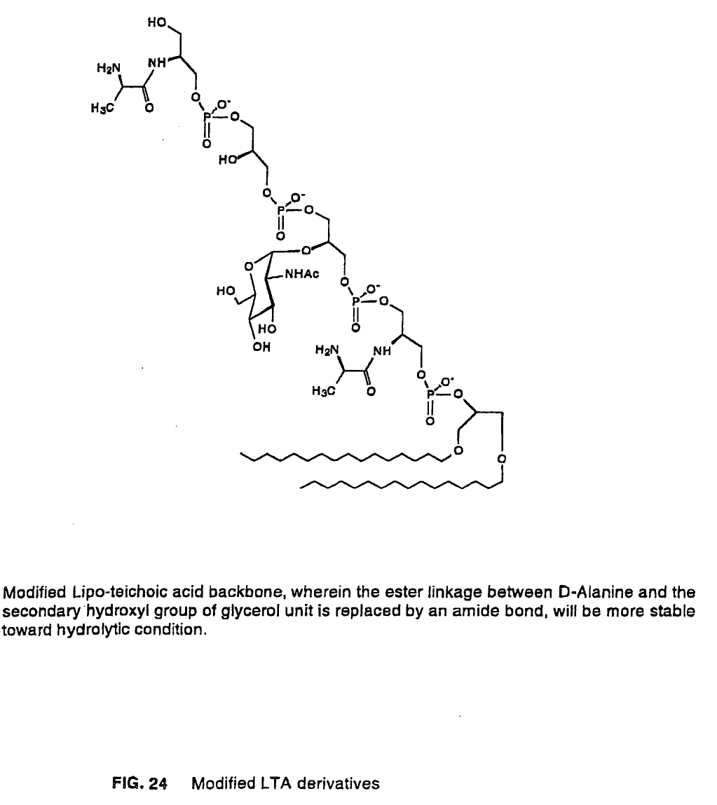

FIG. 25: LTA/GNA and LTA/DNA hybrids.

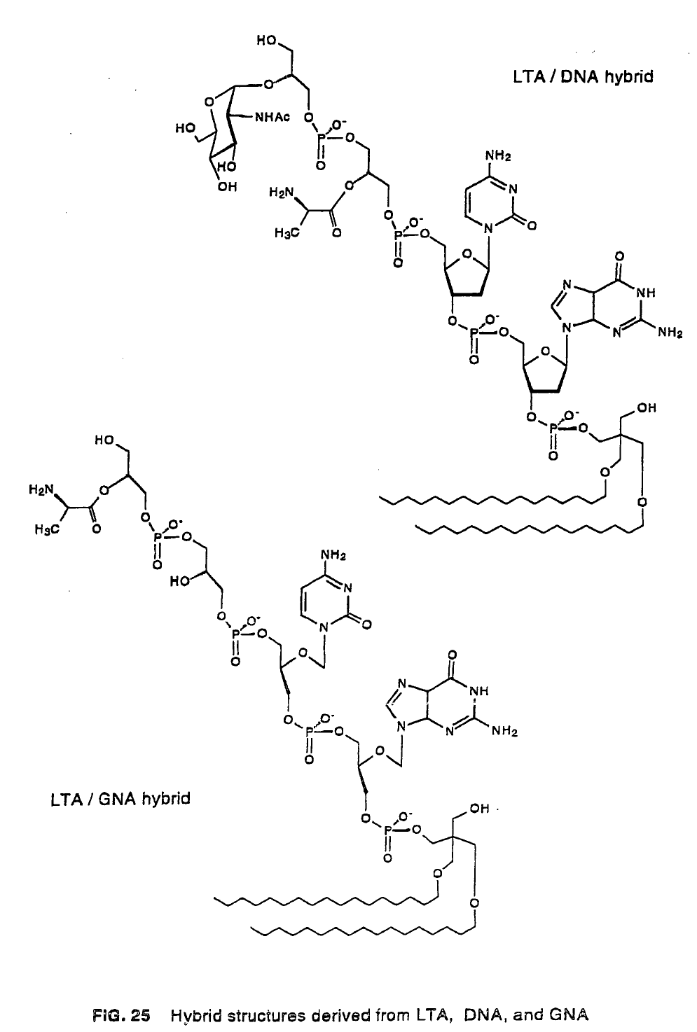

FIG. 26: Oligo (I:C). Lipidated oligo-inosinic and oligo-cytidylic acid may form double stranded complex and mimic the function of dsRNA, which is a ligand for TLR-3. While not shown, the oligonucleotide may additionally comprise CpG, or this oligonucleotide may be used in its own right as an additional adjuvant.

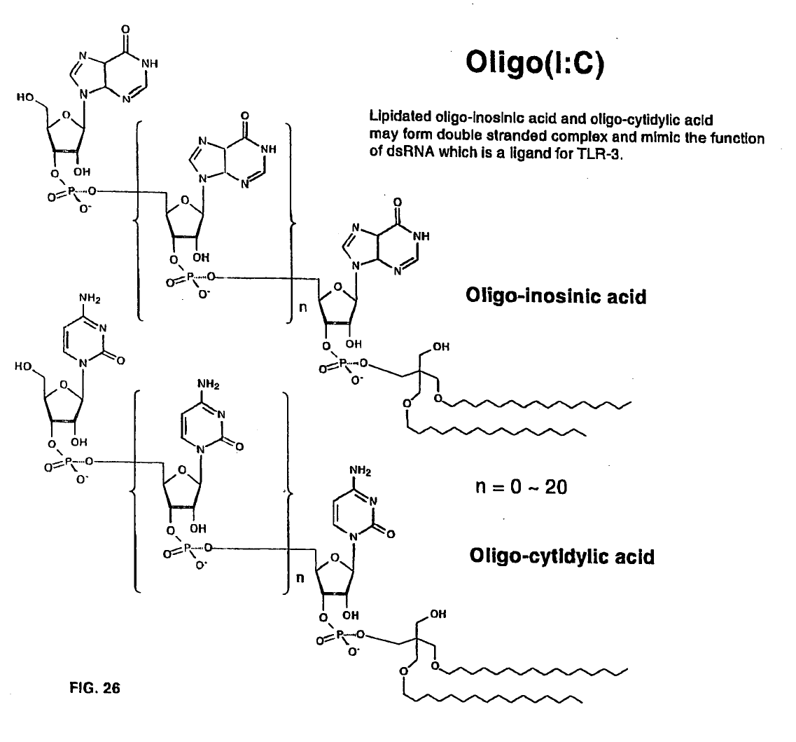

FIG. 27: Oligonucleotide comprising self-complementary regions. In figure, one segment is oligo-inosinic acid and a second segment is oligo-cytidylic acid, joined by a long internucleoside linker. The strand hairpins at the linker to form the double stranded secondary structure. The variable n is 0-6. While not shown, the oligonucleotide may additionally comprise CpG, or this oligonucleotide may be used in its own right as an additional adjuvant.

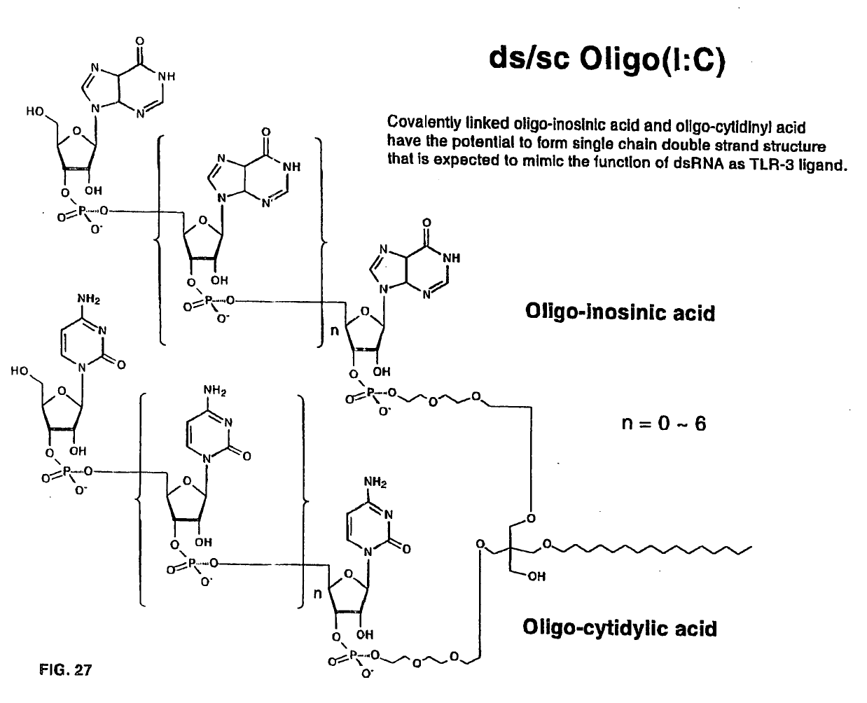

FIG. 28: Oligonucleotide comprising self-complementary regions. Oligonucleotide comprises two I(CI)ₙC segments. These complement each other when oriented and aligned as shown in inset drawing. The variable n is 0 to 4; in the inset drawing, n=3. While not shown, the oligonucleotide may additionally comprise CpG, or this oligonucleotide may be used in its own right as an additional adjuvant.

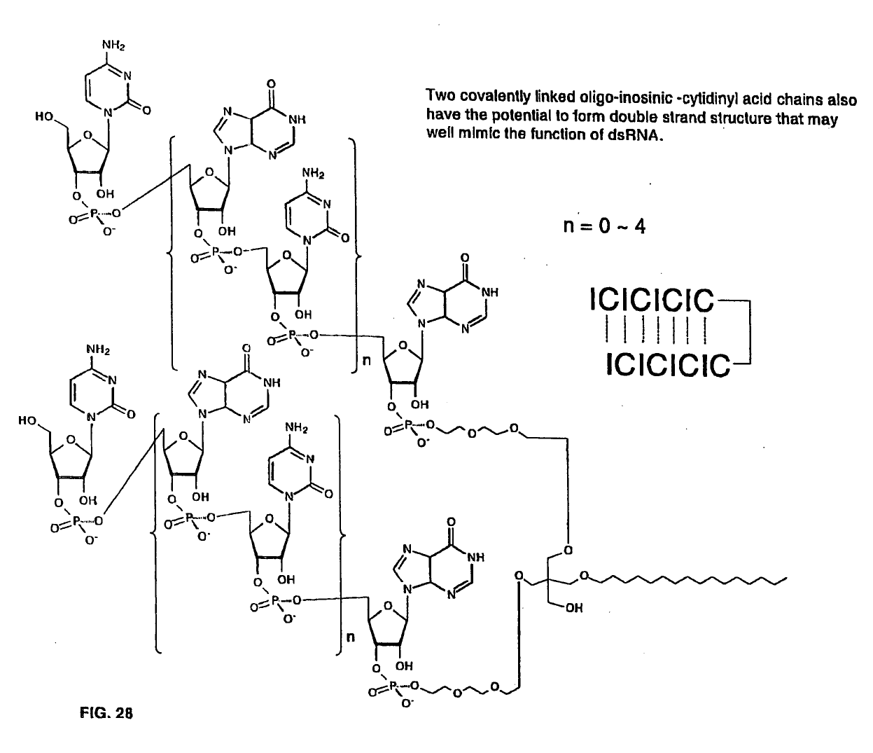

### DETAILED DESCRIPTION OF THE PREFERRED EMBODIMENTS OF THE INVENTION

The immunostimulatory molecules of the present invention are modified oligonucleotides. These oligonucleotides comprise at least one CxG dinucleotide (preferably, a CpG dinucleotide) as defined below, or an analogue thereof, and at least one covalently incorporated lipophilic group. The lipophilic groups(s) may be attached to the free end(s) of the oligonucleotide, or internal to the oligonucleotide. The oligonucleotides may be linear, cyclic or branched, and may include non-nucleic acid moieties (including, but not limited to, lipophilic groups). The nucleotides are not limited to the nucleotides normally found in DNA or RNA.

Molecules

A molecule is a chemical entity consisting of a plurality of atoms connected by covalent or noncovalent bonds. Thus, in a double-stranded DNA molecule, there are two oligonucleotide strands, held together by noncovalent base pairing to form a duplex. This duplex is considered a single molecule. If the duplex is dissociated, then each strand is considered a molecule in its own right.

Linkers and Linking Agents

In discussing the synthesis of molecules, it is helpful to distinguish between a “linker” and a “linking agent”. A “linking agent” is a unitary molecule with at least two reactive functional groups. After reaction with two (or more) target molecules (the same or different), it forms a new molecule of the form “substrate residue-linker-substrate residue”. Thus, the linker is the residue of the linking agent after it has accomplished its mission.

Immunostimulatory Molecules

The term “immunostimulatory oligonucleotide” shall mean an immunostimulatory molecule which comprises at least one oligonucleotide strand.

A molecule is considered immunostimulatory if it stimulates immunocytes in any manner. For example, it may stimulate cytokine (e.g., IL-1, IL-2, IL-4, IL-5, IL-6, IL-7, IL-10, IL-12, IL-15, IFN-gamma, TNF-alpha, G-CSF, GM-CSF, TGF-beta, FLT-3 ligand, CD40 ligand) production by, e.g., lymphocytes; it may stimulate natural killer cell lytic activity; it may stimulate B-cell or T-cell proliferation, antibody production, etc.

Preferably, the molecule causes T-cell proliferation. However, it may immunostimulate the immune system of a subject according to any art-recognized measure of immunostimulatory activity. See the section “Characterizing the Immune Response” of this disclosure, as well as WO98/18810 P20, L15-21, L26-P21, L3 and the references Krieg et al. 1995 (B-cell activation), Alexopoulo et al. 2001 (NF-κB activation, production of type I interferon), Hemml et al., 2000 (cytokine production from macrophages and presensitized lymph nodes), Takeuchi et al. 1999 (production of IL6, nitric oxide and TNFalpha by macrophages; B cell proliferation and MHC class II expression; activation of serine/threonine kinase IRAK); for various measures of immunostimulatory activity.

An immunostimulatory molecule may be immunostimulatory in some respects and immunosuppressive in other respects.

The term “immunostimulatory oligonucleotide” shall mean an immunostimulatory molecule which comprises at least one oligonucleotide strand.

The immunostimulatory oligonucleotides of the present invention may be used in conjunction with other immunostimulatory molecules.

The immunostimulatory oligonucleotides may, but need not, be presented in a liposome.

The immunostimulatory molecule may be used without a pharmaceutically administered immunogen to potentiate the innate immune response to a disease-associated immunogen, whether already presented (in which case the use is a treatment), or which the subject is at risk of experiencing (in which case the use is prophylactic).

The immunostimulatory oligonucleotides may be used in conjunction with a pharmaceutically administered immunogen. The immunogen elicits a specific immune response to one or more epitopes; the immunostimulatory oligonucleotide acts as an adjuvant, potentiating that immune response in a nonspecific way. Hence, the immunostimulatory oligonucleotide may be used with any immunogen.

In some embodiments, the immunostimulatory oligonucleotide and the immunogen are the same molecule, that is, the immunogen comprises the oligonucleotide as a moiety, as well as the specific epitope(s) of interest. The oligonucleotide may then be lipidated either directly (the lipophilic group is directly attached to (incorporated into) a nucleotide), or indirectly, e.g., attached to the peptide moiety.

In a preferred embodiment, the epitope of the immunogen is a MUC1 peptide epitope. If the immunogen comprises the oligonucleotide, it is then especially preferred that the backbone of the oligonucleotide moiety be at least partially a PNA oligomer. Alternatively, the oligonucleotide moiety could be attached to the peptide moiety (comprising the epitope)

Oligonucleotides

An oligonucleotide is an oligomer wherein the monomeric unit is called a nucleotide, or mononucleotide. An oligonucleotide comprises two or more mononucleotides. It may comprise any number of non-nucleotide chemical moieties, and indeed the oligonucleotides of the present invention must at least bear a lipophilic group. The oligonucleotides of the present invention are also heterooligomers, because they comprises a “CxG” dinucleotide unit, or analogue thereof.

An oligonucleotide may be considered to comprise

(1) a series of mononucleotides;

(2) a series of mononucleosides, with each adjacent pair connected by an internucleoside linker; or

(3) a series of nucleobases, with each adjacent pair connected by an interbase linker.

The term “comprise” is used because the oligonucleotide of the present invention must also include at least one lipophilic group, and may also include other chemical moieties, such as one or more amino acid residues, one or more carbohydrates not attached to a nucleobase, and so forth.

Nucleotides

A nucleotide, for the purpose of the present invention, is a monomeric unit which comprises (1) a nucleobase as defined below, and (2) linking means for linking the nucleobase of the instant nucleotide directly or indirectly to the corresponding (but not necessarily identical) linking means of at least one adjacent nucleotide (if any). (This linking means may be called the vertebral element; since, collectively, these vertebral elements are called the backbone.) This definition is deliberately broader than the conventional IUPAC definition of a nucleotide.

The vertebral elements of the oligonucleotide may be the same as that of DNA or RNA, or different, in which case they form what is called an “alternative backbone.” The vertebral elements may be the same for all nucleotides, or they may vary, leading to a hybrid backbone.

If the oligonucleotide strand is linear, as in the case of DNA and RNA, or is cyclic, then the linking means (vertebral element) will be a trivalent means, with one valence connecting to the nucleobase, and the other two valences directly connecting to the linking means (vertebral elements) of the prior and subsequent nucleotides in the sequence, if any (note that the oligonucleotide can be a dinucleotide, in which the vertebral elements will each have one free valence).

If the oligonucleotide strand is branched, then the linking means instead may be a divalent means which links the nucleobase to a branching core means. Each branching core means is linked to at least two vertebral elements, and thus serves to indirectly connect such elements. The demarcation between the linking means of each nucleotide, and the branching core means, may be somewhat arbitrary.

In DNA and RNA, each nucleotide consists essentially of a nitrogenous base (the nucleobase), a sugar, and a phosphate group attached to the 5′ carbon of the sugar. The sequence diversity of DNA and RNA is attributable to position-to-position variation of the nitrogenous base. In DNA, the base is adenine, guanine, cytosine or thymine. In RNA, thymine is replaced by uracil. Collectively, these five bases are referred to herein as normal nucleobases. Abnormal nucleobases may be used and are further discussed below.

The corresponding normal nucleotides, which each further comprises a sugar and a phosphate group, are properly called adenylic acid (AMP), guanylic acid (GMP), cytidylic acid (CMP), thymidylic acid (TMP), and uridylic acid (UMP). However, it is not unusual for them to be identified by reference to the corresponding base (A, C, G, T, U).

For proper nucleic acid nomenclature, see IUPAC-IUB Commission on Biochemical Nomenclature (CBN), “Abbreviations and Symbols for Nucleic Acids, Polynucleotides and their Constituents”, Recommendations 1970, www.chem.gmul.ac.uk/iupac/misc/naabb.html. However, we do at times depart from the conventional nomenclature for simplicity of description of certain analogues of DNA and RNA contemplated herein.

The DNA or RNA sugar is a pentose, with a five-membered ring (four carbons and one oxygen), and is ribose in the case of RNA and 2-deoxyribose in the case of DNA. These are considered normal sugars. Abnormal sugars may be used and are further discussed below. Non-carbohydrate moieties may also be used, in place of sugars.

The base-sugar component of the DNA or RNA nucleotide is called a nucleoside. The normal nucleosides of DNA and RNA are called adenosine, guanosine, cytidine, thymidine and uridine.

In the present invention, the nucleosides may be non-normal. The abnormality may take the form of the use of a non-normal nucleobase, the use of a non-normal sugar-equivalent, or the use of a non-normal attachment of the nucleobase to the sugar equivalent. In DNA and RNA, the nucleobase is connected to the sugar by virtue of a glycosidic bond from the N-3 of a pyrimidine base or the N-9 or a purine base.

The phosphate group provides the normal internucleoside linkage of DNA and RNA. Abnormal internucleoside linkages may be used and are further discussed below.

One may also visualize an interbase linker. In DNA and RNA, the interbase linker is sugar-phosphate-sugar. These interbase linkers are overlapping.

Topology of Interbase and Internucleoside Linkages

The topology of the interbase linkages in DNA and RNA may be described as an inverted “Y” topology. Each nucleobase is directly linked to a single sugar, each sugar is linked to two phosphate groups. The interbase linkers are sugar-phosphate-sugar, and are overlapping. The internucleoside linkers are phosphate groups, and are discrete. The sugars and/or phosphates may of course be replaced by other chemical moieties which preserve the “Y” topology, as in a GNA or PNA oligomer.

The oligonucleotides of the present invention may have other topologies. In a “V” topology, each nucleobase has two attachment sites. The first attachment site of one nucleobase is joined to the second attachment site of an adjacent one by a linker. This interbase linker takes the place of both the sugar and the phosphate in DNA and RNA. The interbase linkers in a “V” topology are discrete. In a “V” topology, the nucleoside is just the nucleobase and the internucleoside linker is the same as the interbase linker.

Nucleobases (Nitrogenous Bases)

The term “nucleobase” refers to a nitrogenous base that can be incorporated into a nucleotide which, in turn, is incorporated into an oligonucleotide.

The nucleobase is preferably a purine or a pyrimidine, although it may be an analogue thereof. In natural DNA, the purine is adenine (A) or guanine (G), and the pyrimidine is cytosine (C) or thymine (T). In natural RNA, uracil (U) appears instead of thymine. Nucleic acids have been prepared in which the nitrogenous base is one other than the five mentioned above, see, e.g., the abnormal bases listed in 37 CFR § 1.822(p)(1).

The pyrimidines have a six-membered ring, and, in DNA and RNA, it is N-3 which is directly bound to the sugar. Substitutions on N-2, C-2, C-4, C-5 and C-6 are possible.

Substituents are functional groups attached to a ring atom. The substituents are preferably fewer than six atoms other than hydrogen. Possible substituents include halogen (fluoro, chloro, bromo, iodo), alkyl, vinyl (—CH═CH2), allyl (—CH2CH═CH2), carboxy (—COO⁻), formyl (—CH═O), hydroxy (—OH), oxy (or oxo) (═O), thio (—SH), sulfono (—SO3H), thioxo (═S), selenoxo (═Se), amino (—NH2), aminooxo (═NH), cyano, nitro (—NO2) and nitroso (—N═O) functional groups, and further substituted forms obtained by combinations thereof, such as haloalkyl, hydroxyalkyl, alkoxy (—OR), aminoalkyl (—NHR or —NR2), alkylallyl, and thioalkyl (—SR). R is preferably methyl, ethyl, isopropyl, n-propyl, isobutyl, n-butyl, sec-butyl or tert-butyl.

There may be zero, one, two, three, four or five independently chosen substituents.

Cytosine is 2-oxy, 4-amino-pyrimidine. Thymine is 2,4-dioxy, 5-methylpyrimidine. Uracil is 2,4-dioxy pyrimidine.

The purines have fused five and six membered rings, and, in DNA and RNA, it is the N-9 which is directly bound to the sugar. Substitutions on N-1, X-2, N-3, C-8, N-7, and C-6 are possible. The possible substituents are the same as for the pyrimidines. There may be 0-6 substituents.

Adenine is 6-Amino-purine. Guanine is 2-Amino-6-oxy-purine.

Alternative purines of particular interest include hypoxanthine (6-oxypurine), xanthine (2,6-dioxypurine), and orotic acid (2,4-dioxy, 6-carboxypurine).

In addition, one may have aza (N replaces C), deaza (C replaces N),

CxG Dinucleotide Units

The olionucleotides of the present invention comprise at least one “CxG” dinucleotide or an analog thereof. The term “CxG” dinucleotide indicates that a first nucleotide which comprises the base cytosine (C) is attached, by a linker denoted “x”, to a second nucleotide which comprises the base guanine (G). It is not necessary that the linker “x” comprise a phosphate group or that the nucleotides comprise a sugar moiety.

The “CxG” dinucleotide is preferably a “CpG” dinucleotide. The term “CpG” dinucleotide indicates that a first nucleotide which comprises the base cytosine (C) is attached, by a linker comprising a phosphate group (p), to a second nucleotide which comprises the base guanine (G). It is not necessary that the nucleotides be phosphate-sugar nucleotides as in DNA or RNA. If they are DNA or RNA nucleotides, then it will be understood that the orientation is 5′-CpG-3¹.

Alternatively, the cytosine of the CxG (including CpG) dinucleotide may be replaced with a cytosine analogue, which is a pyrimidine more similar to cytosine than to thymine or uracil. Cytosine differs from thymine and uracil as follows: (1) at the 1-6 positions, cytosine has —N═C(NH2)— and thymine (and uracil) have —HN—C(═O)—; (2) at position 5, cytosine is unsubstituted and thymine is methylated. A cytosine analogue preserves these distinctions. However, the cytosine analogue is preferably not 5-methylcytosine, as this methylation is known to greatly reduce the immunostimulatory effect in unlipidated CpG oligonucleotides.

Likewise, the guanine of the CxG (including CpG) may be replaced with a guanine analog, which is a purine more similar to guanine than to adenine. Guanine differs from adenine in that (1) at the 1-6 positions, guanine has —HN—C(═O)— and adenine has —N═C(NH2)—; and (2), at the 2 position, guanine is aminated and adenine is unsubstituted. A guanine analogue preserves these distinctions. Preferably, the guanine analogue is not a methylated derivative of guanine.

If either or both of these replacements are made, we obtain an analogue of the “CxG” (or “CpG”) dinucleotide.

The CxG dinucleotide may have a standard DNA or RNA backbone (which is a special case of the CpG dinucleotide), or it may have an alternative backbone. The alternative backbone may be confined to the CpG linkage, or it may occur elsewhere in the oligonucleotide as well.

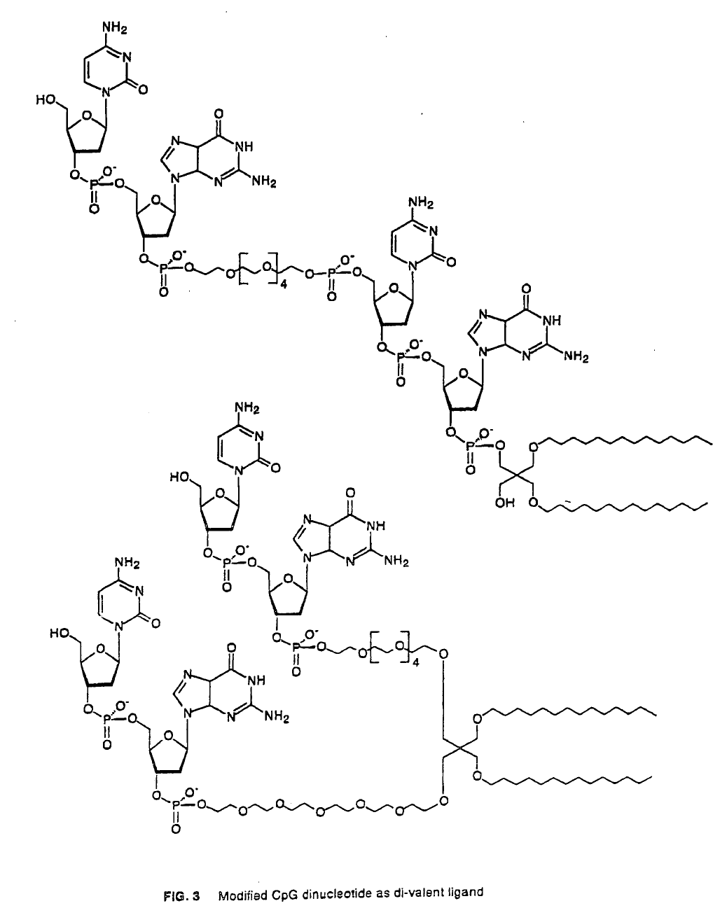

The immunostimulatory oligonucleotide may comprise a plurality of CxG (especially CpG) dinucleotides. See, e.g., FIG. 3. The CxG may be adjacent or non-adjacent.

Overall Base/Nucleoside Sequence

We here use the symbols A, C, G, T and U merely to identify a nucleoside comprising a particular nucleobase; the nucleoside does not necessarily comprise ribose or 2-deoxyribose as in DNA or RNA.

The bases, if any, flanking the CxG on its 5′ end (or what would be considered the 5′ end if the oligonucleotide were DNA) are preferably AT, GA, or GT. The bases, if any, flanking the CxG on its 3′ end (or what would be considered the 3′ end if the oligonucleotide were DNA) are preferably TA, TT, or AT.

These flanking bases may be modified (non-normal) bases as taught by WO01/12804.

In the embodiments in which the CxG dinucleotide is flanked by other nucleotides, the most preferred sequences are those comprising one or more copies of

(A) a K motif, which activates monocytes and B cells, and stimulates secretion of IL-6, such as TCGTA or TCGTT, and

(B) a D motif, which activates NK cells and secretion of IFN-gamma, such as a palindromic sequence like ATCGAT.

Sequences of particular interest include GTCGTT (optimal in human), GACGTT (optimal in mouse), and

| | |
|---|---|
| GGTGCATCGATGCAGGGGGG. | (SEQ ID NO: 3) |

The oligonucleotide may be T-rich as taught by WO01/97843.

A special case of nucleobase modification is that of a thymine dimer. In DNA, two adjacent thymidine nucleosides (TT) may dimerize in situ so that the single six membered ring of each is joined into a new fused polyaromatic structure having two six membered rings joined by a fused four membered ring. This is called a thymidine dimer. However, since the same reaction could occur between two non-normal nucleosides having thymine nucleobases, it may more generally be called a thymine dimer.

Thymidine dimerization occurs in nature as a result of ultraviolet damage to DNA. It is believed that the immune system is sensitive to the formation of thymidine dimers for this reason. Hence, by deliberately incorporating a thymine dimer into the base sequence, we can enhance the immune response elicited by the oligonucleotide. Formation of a thymine dimer can be represented by

|\overline{ }|
T T

Oligonucleotide Length

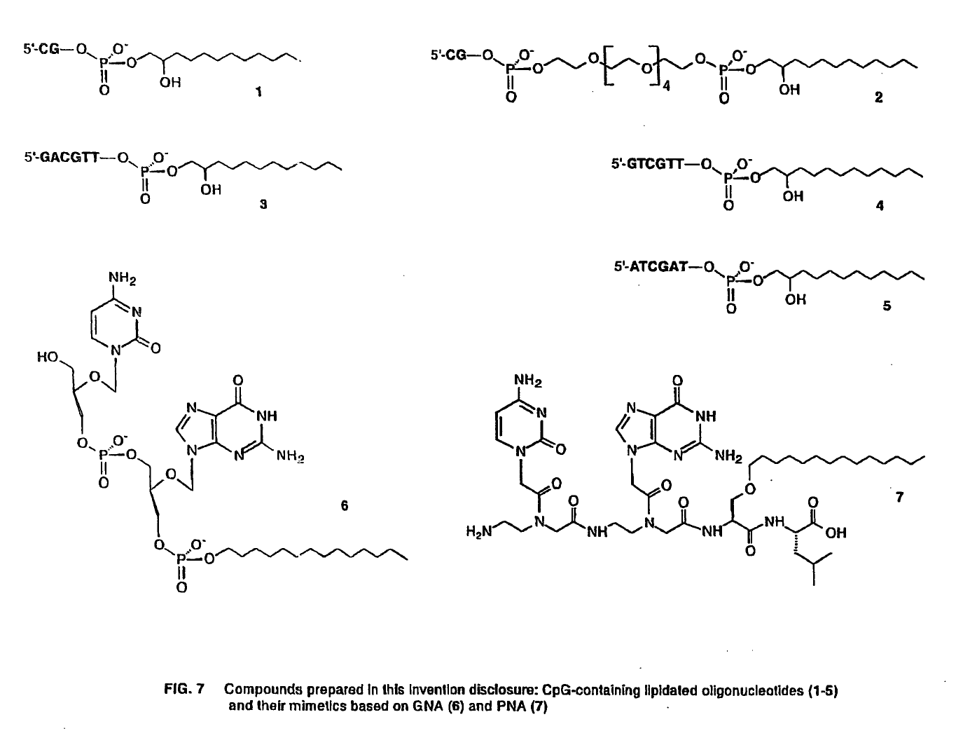

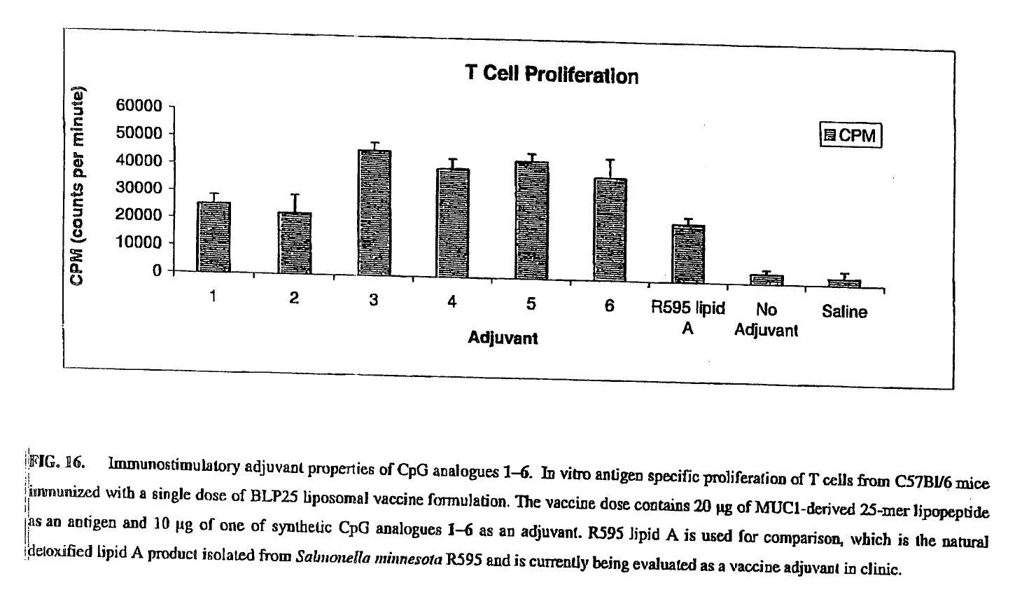

The art has generally taught that immunostimulatory oligonucleotides comprise CpG and are at least five, six or eight bases in length (per strand, if double stranded). While the present invention includes these ranges as embodiments (we distinguish the art in that we further teach inclusion of a lipophilic group), we have found that even a dinucleotide, when lipidated, has activity (cp. FIGS. 7 and 16). Hence, the present invention also includes oligonucleotides wherein the number of nucleobases on one strand is less than eight, i.e., two, three, four, five, six or seven.

The maximum number of nucleobases in the oligonucleotides of the present invention is 100, more preferably 50, still more preferably 30, even more preferably 20, most preferably 10. The preferred embodiments of the invention include any rational combination of the preferred minima and preferred maxima set forth above.

The lipidated oligonucleotides with fewer bases than those of the unlipidated oligonucleotides used by the art, i.e., those with 2-5 or 2-4 bases, are of particular interest.

Strandedness

The molecules of the present invention may be single- or double-stranded. To be double stranded, the two strands must feature sequences of substantially complementary bases (A:T, C:G, etc.) and the backbones must be compatible with the formation of a stable duplex.

A molecule is considered to be double stranded if it has two strands which are at least partially complementary. Hence, a double stranded molecule may be partially double stranded, or completely double stranded. A molecule may be a partially double stranded molecule because one strand is longer than the other, or because aligned bases in the strands do not hydrogen bond to each other.

A molecule has double stranded structure if (1) it is an at least partially double stranded molecule, or (2) it is at least partially self-complementary and thereby has a stable double stranded secondary structure as a result of the folding of a single strand.

When the molecule is at least partially double stranded, in a preferred embodiment, one strand comprises two or more hypoxanthines (hypoxanthine is the base corresponding to the nucleoside inosine, I.) and the other strand comprises two or more complementary cytosines. More preferably, there are no more than 21 such base pairs. The resulting oligo (I:C) moiety is intended to mimic the TLR-3 ligand activity of dsRNA.

This I:C pairing can be achieved in a number of ways. For example, one strand may comprise Iₙ and the other Cₙ. Another double stranded embodiment of interest is one in which one strand comprises (IC)ₙ, and the other, (CI)ₙ, so that I:C base pairing occurs. Other arrangements of I and C to achieve I:C base pairing are possible.

If the molecule will be used in single stranded form, it may be designed to include regions which are self-complementary to each other, so that the oligonucleotide will tend to fold so that these regions form secondary structures. These secondary structures may protect the nucleotide from enzymatic degradation.

While the strands of a double stranded molecule are normally held together by noncovalent bonds (Watson-Crick base pairing as a result of hydrogen bonding), it is possible to stabilize the duplex by an internucleoside linkage which joins a 5′ end of one strand to the proximal 3′ end of the other strand (after which the ends in question are no longer free ends). This can be done with just one pair of ends, or with both of them. In like manner, a single stranded molecule which folds as a result of self-complementarity can have its folded structure stabilized by such a linkage. In either case, a lipophilic group may be incorporated into this linkage.

Alternatively, the molecule may be single stranded and designed to avoid formation of stable secondary structures.

In general, double stranded structure provides greater resistance to degradation during delivery and single stranded structure provides greater innate activity once delivered successfully.

Alternative Backbones

The oligonucleotides of the present invention may, but need not, be characterized by the occurrence of one or more “alternative backbones” (i.e., backbones other than those of DNA or RNA) in some or all of the oligonucleotide. Alternative backbones may be used to make the oligonucleotide more resistant to enzymatic degradation, or to achieve other useful effects.

An alternative (abnormal) backbone may be provided by introducing one or more abnormal internucleoside linkages, or by replacement of the normal sugar with an abnormal moiety.

DNA, RNA and LTA are basically di-phosphate ester polymers with slightly different building units (deoxyribose for DNA, ribose for RNA, and glycerol for LTA), therefore, as PAMPs they are structurally closely related to each other. Since their immune activation properties are all mediated by the same group of receptors—TLRs, it is reasonable to group these TLR ligands into the same type of molecular structures. Furthermore, it seems that the ligand specificity requirement of TLRs is relatively low; thus, we perceive that the hybrid structures of DNA, RNA, LTA, and/or glycerol-based nucleic acid (GNA) might well be potential ligands for multiple TLRs.

Sugar and Phosphate Replacements

The normal vertebral element is composed of a “sugar-equivalent” and a “phosphate equivalent”. The “sugar-equivalent” is any chemical moiety that serves the same structural role as the sugar moiety of DNA or RNA, i.e., linking the nucleobase to the “phosphate equivalent”. It includes, but is not limited to, the normal sugar moieties 2-deoxyribose and ribose. Likewise, the “phosphate equivalent” is any chemical moiety that serves the same structural role as the phosphate moiety of DNA or RNA, i.e., linking the “sugar equivalents” of adjacent nucleosides. It includes, but is not limited to, the normal phosphate moiety.

In general, there is no difficulty identifying the sugar equivalent if the phosphate equivalent remains phosphate, or identifying the phosphate equivalent if the sugar equivalent remains a sugar. However, if both are varied simultaneously, then an arbitrary demarcation is necessary.

The following is suggested: (1) identify the longest chain of atoms in the oligonucleotide strand (or segment thereof, if it is segmented and branched) in question; (2) identify the shortest chain of atoms leading from the nucleobase in question to that chain of (1); and (3) identify the point at which chain (2) is attached to chain (1). The sugar equivalent corresponds to all of chain (2), including any side groups and the attachment site, plus any atom of chain (1) which is (a) a member of the same ring as the attachment site atom (DNA and RNA attach to the nucleobase to the main chain of the oligonucleotide via two ring atoms of the sugar), or (b) immediately adjacent to the attachment site and not a group VI atom (e.g., oxygen).

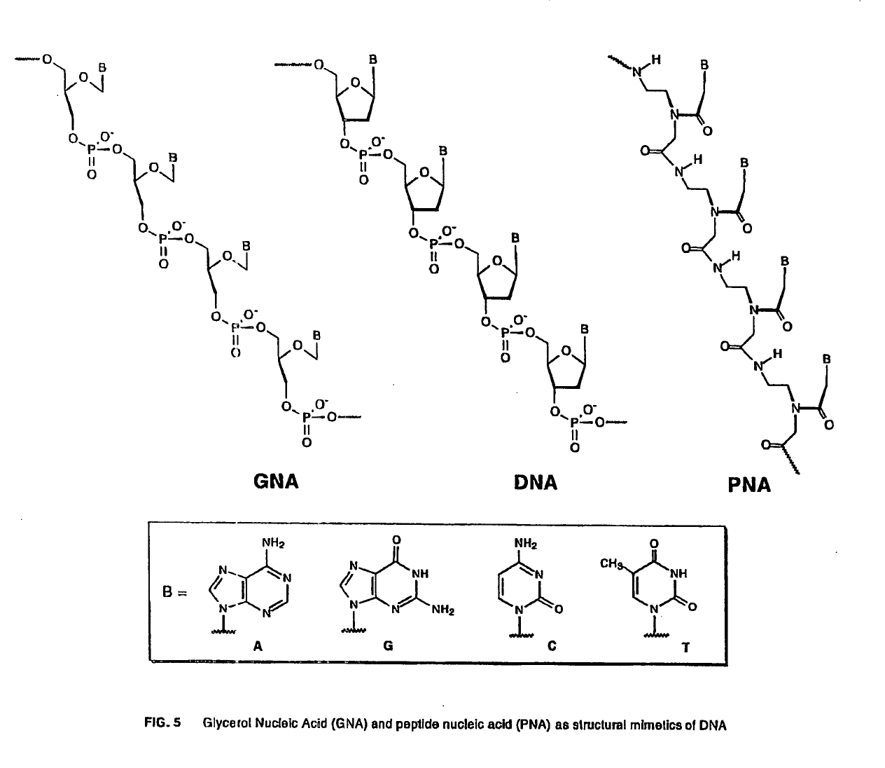

By this definition, the sugar equivalent in DNA and RNA is simply the sugar; the sugar equivalent in the glycerol nucleic acid of FIG. 5 is the glycerol residue —CH2—CH(—O—)—CH2—; and the sugar equivalent of the peptide nucleic acid of FIG. 5 is the —CH2—N(—C(═O)CH2—)—CH2—.

The phosphate equivalent can then be identified as the remainder of the vertebral element; it is the phosphate group for the DNA and GNA of FIG. 5, and the —C(═O)—NH—CH2- for the PNA of FIG. 5. These are also the internucleoside linkers for those molecules. That is, the internuceloside linkage joins the sugar equivalent of one nucleoside to the sugar equivalent of another.

Where the oligonucleotide has a V-topology, the interbase linker may be considered both the sugar-equivalent and the phosphate-equivalent for the purpose of the claims.

Internucleoside Linkages

The normal internucleoside linkage is

$$\begin{matrix}{3}^{′} O \\ {O}^{-} - P = O \\ O {5}^{′}\end{matrix} .$$

Note that in this structure, the oxygen labeled 3′ is the one which, in DNA and RNA, attaches to the 3′ carbon of a nucleoside sugar, and the one labeled 5′ is the one which likewise attaches to the 5′ carbon of another nucleoside sugar. These two oxygens, together with the phosphorus atom, may be called the main chain atoms, because they lie on the longest chain of the oligonucleotide (or oligonucleotide segment, if the molecule has segments and is branched). The other two oxygens are side chain atoms as they are not part of the most direct connection between nucleosides.

Variations of the inter-nucleoside linkage can be characterized as (1) those which affect only the main chain, (2) those which affect only side chains, and (3) those which affect both.

Where there is a side chain modification, the number of non-hydrogen atoms in the side chains may remain unchanged. For example, the doubly bonded oxygen may be replaced by ═S, ═Se, or ═Te, and/or the singly bonded oxygen may be replaced by a monovalent radical with a single non-hydrogen atom such as —CH3, —NH2, —OH, —SH, —SeH, —TeH, or —X.

Alternatively, one or more of the side chains may be a larger substituent. Such substituents include, but are not limited to, those mentioned in connection with nucleobases. The substituent is preferably an organic constituent composed of not more than 50 non-hydrogen atoms (more preferably not more than 25, still more preferably not more than 10, most preferably not more than 5), selected from the group consisting of carbon, silicon, oxygen, nitrogen, sulfur, selenium, tellurium, phosphorus, and boron. The substituent may be, or include, a lipophilic group, especially a strongly or highly lipophilic group as defined elsewhere in this disclosure.

Where there is a main chain modification, the number of non-hydrogen atoms may remain unchanged. Thus, either oxygen (—O—) may be replaced independently with —CH2-, —NH—, —S—, —Se—, or —Te—.

Likewise, the phosphorus atom may be replaced. If the valency of the replacement atom is less than that of the phosphorus atom, this will compel elimination or modification of the side chain oxygens. The replacement atom may be, e.g, boron, carbon, silicon, or nitrogen.

The main chain may also be modified in order to lengthen it, with or without retaining the original oxygen and phosphorus atoms thereof. Such modifications involve inclusion of one or more lengthening moieties, which preferably are independently chosen from the group consisting of —CH2-, —CHZ-, —NH—, —NHZ-, —O—, —C(═O)—, —C(═S)—, —C(═Se)—, —C(═Te)— and —PO4-, where Z is a halogen or an organic constituent composed of not more than 50 non-hydrogen atoms (more preferably not more than 25, still more preferably not more than 10, most preferably not more than 5) selected from the group consisting of carbon, silicon, oxygen, nitrogen, sulfur, selenium, tellurium, phosphorus, and boron.

Alternative linkages include the following:

$$\begin{matrix}{3}^{′} O \\ {S}^{-} - P = O \\ O {5}^{′}\end{matrix}$$

$$\begin{matrix}{3}^{′} O \\ {CH}_{3} - P = O \\ O {5}^{′}\end{matrix}$$

$$\begin{matrix}{3}^{′} O \\ {NR}_{2} - P = O \\ O {5}^{′}\end{matrix}$$

(where the Rₛ are hydrogen and/or alkyl)

$$\begin{matrix}{3}^{′} O \\ RO - P = O \\ O {5}^{′}\end{matrix}$$

(where R is hydrogen or alkyl)

$$\begin{matrix}{3}^{′} O \\ {S}^{-} - P = S . \\ O {5}^{′}\end{matrix}$$

Possible replacements for the 3′-O—P—O-5′ main chain include 3′-O—CH₂C(═O)—O-5′,3′-O—C(═O)—NH-5′, and 3′-CH₂CH₂CH₂S—CH₂-5′.

The entire nucleic acid molecule may be formed of such modified linkages, or only certain portions may be so affected.

Nucleic acid molecules suitable for use in the present invention thus include but are not limited to methylphosphonates, see Mill, et al., Biochemistry, 18:5134-43 (1979), phosphorothioates, see Matsukura, et al., Proc. Nat. Acad. Sci., 84:7706-10 (1987), oligodeoxynucleotides covalently linked to an intercalating agent, see Zerial, et al., Nucleic Acids Res., 15:9909-19 (1987), oligodeoxynucleotide conjugated with poly(L-lysine), see Leonetti, et al., Gene, 72:32-33 (1988), and carbamate-linked oligomers assembled from ribose-derived subunits, see Summerton, J., Antisense Nucleic Acids Conference, 37:44 (New York 1989). Boranophosphates, formacetals, siloxanes, dimethylenethiolates, sulfoxidates and sulfonates are also known in the art.

For a general review, see Uhlmann and Peyman, ‘Antisense Oligonucleotides: A New Therapeutic Principle,” Chem. Revs., 90:544-84 (1990). They discuss oligonucleotides with a modified internucleotide phosphate residue in which a phosphate oxygen not involved in the bridge is replaced (methylphosphonates, phosphorothioates, phosphorodithioates, phosphoroamidates, and phosphate esters), oligonucleotides in which a phosphate oxygen involved in the bridge is replaced (bridged phsophoramidates, bridged phosphorothioates, bridged methylenephosphates), and more radical phosphate replacements such as siloxane, carbonate, carboxymethylester, acetramidate, carbamate, and thioether bridges.

They also discuss the possibility of replacing both the sugar and the phosphate with a synthetic polymer, e.g., poly(N-vinyl), poly(methacryloxyethyl), poly(methacrylamide), and poly(etheylenimine). In some embodiments of the invention, at least one of the internucleoside linkages is one of these structures. In other embodiments of the invention, none of the internucleoside linkages are one of these structures.

Intranucleoside Modifications

It is also possible to replace the ribose (RNA) or 2-doxyribose (DNA) sugar with another moiety which is at least trifunctional (it must bind the nucleobase, and two phosphate groups or replacements thereof). The moiety may be another carbohydrate (sugar), or an unrelated moiety.

If the moiety is a carbohydrate, it is preferably a monosaccharide. Monosaccharides are polyhydroxy aldehydes (H[CHOH]ₙ—CHO) or polyhydroxy ketones (H—[CHOH]ₙ—CO-[CHOH]ₘ—H) with three or more carbon atoms, or derivatives thereof such as those discussed below. It is preferably a triose, tetrose, pentose, hexose, heptose or octose, or derivative thereof, with pentoses (5 carbons) or hexoses (6 carbons), and their derivatives, being more preferred. Ribose is a pentose.

Each monosaccharide unit may be an aldose (having an aldehydic carbonyl or potential aldehydic carbonyl group) or a ketose (having a ketonic carbonyl or potential ketonic carbonyl group). If it is a ketose, the position of the ketonic carbonyl (or potential ketonic carbonyl, for a ketose derivative) may vary.

The monosaccharide unit further may have more than one carbonyl (or potential carbonyl) group, and hence may be a dialdose, diketose, or aldoketose. The term “potential aldehydic carbonyl group” refers to the hemiacetal group arising from ring closure, and the ketonic counterpart (the hemiketal structure).

In some preferred embodiments, the monosaccharide unit is a cyclic hemiacetal (cyclized aldose) or hemiketal (cyclized ketose). Cyclic forms with a three membered ring are oxiroses; with four, oxetoses, with five, furanoses; with six, pyranoses; with seven, septanoses, with eight, octanoses, and so forth. The locants of the positions of ring closure may vary.

The monosaccharide may be linear or cyclic, with cyclic being preferred. Cyclic forms with 3-8 ring atoms are preferred; furanoses and pyranoses are especially preferred. Ribose is a pyranose (one ring atom is oxygen).

The monosaccharide unit may further be, without limitation, a deoxy sugar (alcoholic hydroxy group replaced by hydrogen), amino sugar (alcoholic hydroxy group replaced by amino group), a thiosugar (alcoholic hydroxy group replaced by thiol, or C═O replaced by C═S, or a ring oxygen of cyclic form replaced by sulfur), a seleno sugar, a telluro sugar, a C-substituted monosaccharide, an unsaturated monosaccharide, an aza sugar (ring carbon replaced by nitrogen), an amino sugar (ring oxygen replaced by nitrogen) an alditol (carbonyl group replaced with CHOH group), aldonic acid (aldehydic group replaced by carboxy group), a ketoaldonic acid, a uronic acid, an aldaric acid, and so forth.

Carbohydrate derivatives of particular interest include alditols, preferably of 3-8 carbon atoms, especially ribitol and glycerol. Ribitol is the five carbon alditol corresponding to ribose. Glycerol has three carbons. The nucleobase may be attached, directly or indirectly, to any of the carbons not involved in the intersugar linkages.

RNA analogues of particular interest include those in which the ribonucleotides are 2′ modified, e.g., 2′ O—methyl derivatives.

When the nucleoside contains sugar, the internucleoside linkages may, but need not, be 3′ to 5′ linkages as in DNA or RNA. For example, linkages could be alternately 5′ to 5′ and 3′ to 3′. Or they could involve carbons other than the 3′ and 5′ carbons.

The sugar may also be replaced by a non-carbohydrate moiety, or eliminated without replacement.

For a review of some of the more interesting structures, see Leumann, “DNA Analogues: From Supramolecular Principles to Biological Properties,” Bioorganic & Medicinal Chem., 10: 841-54 (2002). The structures listed include 4′-6′ linked hexopyranosyl-NAs, 2′-4′ linked pentopyranosyl-NAs, 3′-4′ linked pentopyranosyl-NAs, hexitol-NAs, locked nucleic acids (e.g., beta-D-Ribo-LNA), and bicyclo- and tricyclo-DNAs.

Peptide Nucleic Acid Oligomers

A category of alternative backbones of particular interest are those in which the internucleoside linkage comprises a peptide (—NHCO—) bond, as in a PNA oligomer (see FIG. 5, and FIG. 7 compound 7). A PNA oligomer is here defined as a series of contiguous nucleotides wherein the internucleoside linkages comprise peptide (—NHCO—) bonds.

The classic PNA oligomer is composed of (2-aminoethyl)glycine units, with nucleobases attached by methylene carbonyl linkers. That is, it has the structure
H—(—HN—CH₂—CH₂—N(—CO—CH₂—B)—CH₂—CO—)ₙ—OH
where the parenthesized substructure is the PNA monomer.

In the PNA oligomer, the nucleobase B is separated from the backbone N by three bonds, and the points of attachment of the side chains are separated by six bonds. The bases A, G, T, C and U are preferred.

A PNA oligomer may further comprise one or more amino acid residues, especially glycine and proline.

One can readily envision related molecules in which (1) the —COCH2- linker is replaced by another linker, especially one composed of two small divalent linker elements as defined below, (2) a side chain is attached to one of the three main chain carbons not participating in the peptide bond (either instead or in addition to the side chain attached to the N of the classic PNA); and/or (3) the peptide bonds are replaced by pseudopeptide bonds.

A peptide bond has two small divalent linker elements, —NH— and —CO—. Thus, a preferred class of psuedopeptide bonds are those which consist of two small divalent linker elements. Each may be chosen independently from the group consisting of amine (—NH—), substituted amine (—NR—), carbonyl (—CO—), thiocarbonyl (—CS—), methylene (—CH₂—), monosubstituted methylene (—CHR—), disubstituted methylene (—CR1R2-), ether (—O—) and thioether (—S—). The more preferred pseudopeptide bonds include:

N-modified —NRCO—

Carba Ψ —CH₂—CH₂—

Depsi Ψ —CO—O—

Hydroxyethylene Ψ —CHOH—CH₂—

Ketomethylene Ψ —CO—CH₂—

Methylene-Oxy —CH₂—O—

Reduced —CH₂—NH—

Thiomethylene —CH₂—S—

Thiopeptide —CS—NH—

Retro-Inverso —CO—NH—

A single molecule may include more than one kind of pseudopeptide bond.

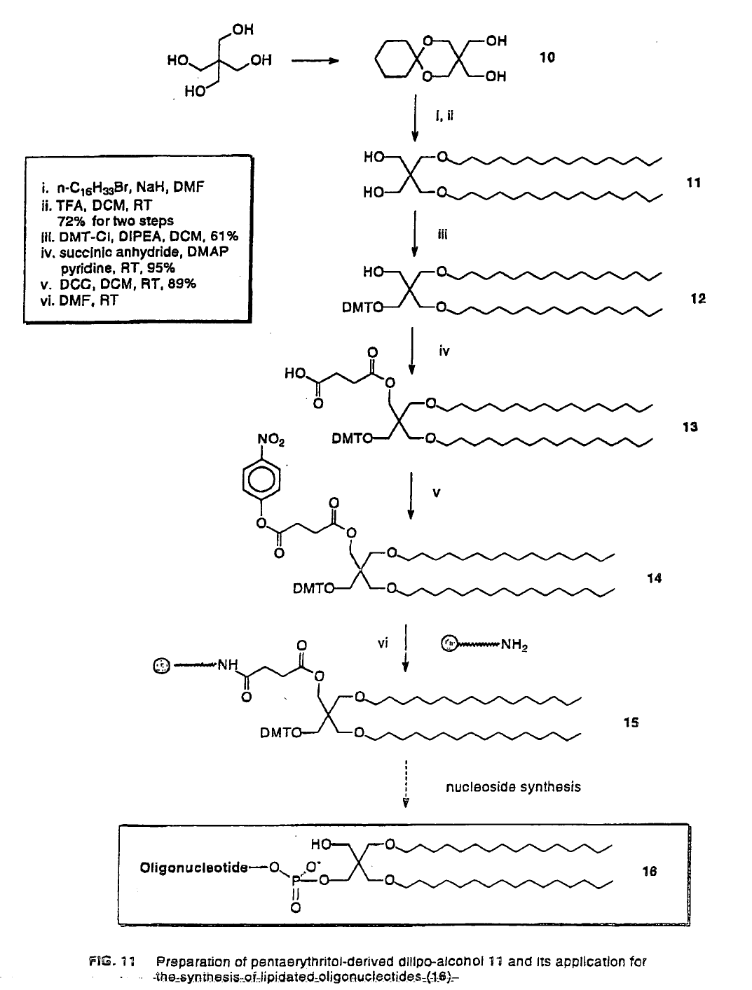

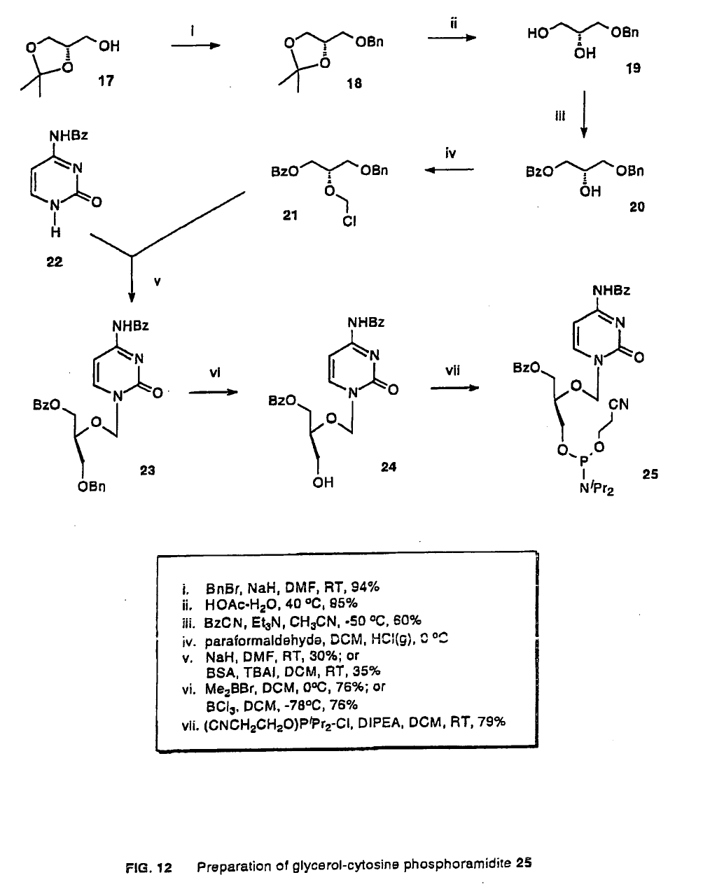

Uhlmann, et al., “PNA: Synthetic Polyamide Nucleic Acids with Unusual Binding Properties,” Angew. Chem. Int. Ed. 37:2796-2823 (1998) describe several PNA analogues. These include phosphonic ester nucleic acids (with N-(2-hydroxyethyl)aminomethyl phosphonic acid backbones), PNA analogues with backbones bacsed on ornithine, proline, diaminocyclohexane, and the phosphoramidate of 2-aminopropanediol, and PNA analogues in which the central amide bond is replaced by a configurationally defined C═C double bond. See monomers A-O in his FIGS. 11 and 12.

It may be particularly advantageous to use, at least in part, a PNA oligomer backbone, when the oligonucleotide is to be engineered to further include a peptide epitope.

Glycerol Nucleic Acid Oligomers

Polyol-derived nucleic acid oligomers are also of interest. The polyol replaces the sugar of a conventional oligonucleotide. Glycerol itself is 1,2,3-propanetriol. However, other polyols may also be of interest. If the polyol has more than three carbons, the interbase spacing will increase. Polyols with 3-6 carbons are preferred. We will use the term “GNA oligomers” to encompass use of the higher polyols, too.

Leumann notes that the 3′ methyl analogue (1′,2′-Seco DNA) of the glycerol-DNA has been made. Neither readily form duplexes.

In 1978 Zamecnik and Stephenson (Zamecnik & Stephenson, 1978) first proposed the use of synthetic oligo-nucleotides for therapeutic purpose. The major problems associated with this principle are the instability of the oligo-nucleotides towards extra- and intracellular enzymes and the difficulty in penetrating through the cell membrane (Uhlmann & Peyman, 1990). For that reason, alternative chemically modified oligo-nucleotides have been prepared as anti-sense oligo-nucleotides to achieve higher stability towards various enzymes and higher ability to penetrate cell membranes. In the same way, GNA is expected to be more stable towards nucleases, and through lipophilic modification GNA is likely to penetrate the cell membrane with more ease.

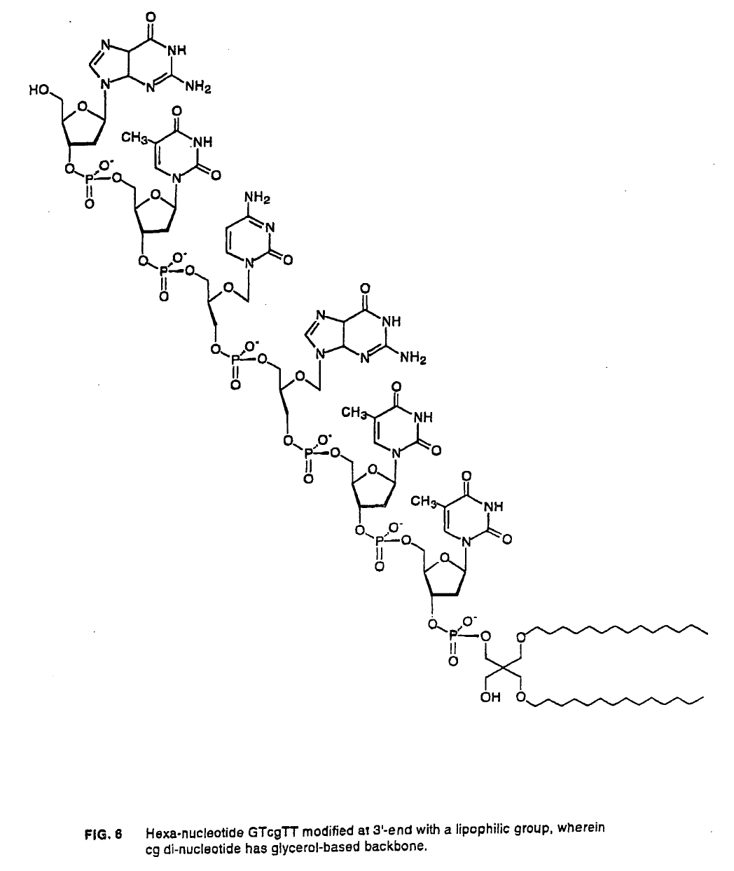

In some embodiments, the normal phosphate internucleoside linkage is retained. As can be seen in FIG. 6, the glycerol linking agent becomes a trifunctional linker of the form —CH₂—CH₂(—O—)CH₂—, with the C-2 carbon being linked through the —O— to a nucleobase. See also FIG. 7, compound 6.

In other embodiments, the normal phosphate linkage is replaced by one of the form

-phosphate-linker Z-phosphate.

In these embodiments, linker Z is preferably aliphatic, more preferably of the form -[(small alkyl)-O—]ₙ, where “small alkyl” is not more than six carbons, and n is 1 to 20. Still more preferably, the small alkyl is —CH₂CH₂—, and/or n=6, as shown in FIG. 3, and in FIG. 7 compound 2.

Segmented Oligonucleotides

Segments are demarcated by the presence of long internucleoside linkages, as defined below.

A short internucleoside linkage is a linkage having a main chain of not more than five atoms (in DNA and RNA, the normal linkage is a phosphate group, having a main chain of three atoms, O—P—O). By this definition, the normal PNA internucleoside linkage, —C(═O)—NH—CH₂—, is a short linkage as its main chain has a length of only three atoms (C—N—C).

A long internucleoside linkage is any linkage between nucleosides that does not qualify as a short linkage. Thus, the linkages shown in FIG. 3 are long linkages. As shown in FIG. 3, the long internucleoside linkage may include at least one lipophilic group.

A segment of the oligonucleotide is defined as (1) a consecutive series of two or more nucleosides within which adjacent pairs are connected by short internucleoside linkages, or (2) a single nucleoside whose only connection to other nucleosides is by long internucleoside linkage(s).

Thus, in FIG. 3, we see two segment oligonucleotides of the form CpG-long linkage-CpG, where each CpG is considered a segment. A structure of the form CxG-long linkage-CxG, where the “x” was a short linkage, would also be a two segment oligonucleotide. A structure of the form GpApCpGpTpT-long linkage-GpApCpGpTpT, ii.e., one in which the hexanucleotide of compound 1 (FIG. 7) replaced each CpG of the FIG. 3 compounds, would likewise be a two segment oligonucleotide. GxAxCxGxTxT-long linkage-GxAxCxGxTxT, wherein the “x”'s are all short linkages (and may be the same or different), would be a two segment oligonucleotide.

In many biological systems, the effect of a ligand is enhanced if it is presented in multiple copies as part of a single molecule. For example, immunogens have been constructed in the form of linear molecules with clustered epitopes, see WO98/46246, or of branched molecules presenting several epitopes (e.g., the MAP-4, where four epitopes are attached to a branched lysine core).

In a preferred embodiment, the oligonucleotide comprises at least two segments, and at least two of the segments each comprise at least one CxG dinucleotide unit. Desirably, all of the segments each comprise at least one CxG dinucleotide unit.

Preferably, at least one pair of CxG-containing segments are connected, directly or indirectly, by a long internucleoside linkage. Thus, we have, in the simplest case, (CxG containing segment)-long linkage-(CxG containing segment). We could also have, e.g., (CxG containing segment)-long linkage-(other segment)- long linkage-(CxG containing segment).

The multisegmented oligonucleotide of the present invention may have more than two segments. In this case, it can be linear, i.e., segment (-long linkage-segment)ₙ, where n is at least two and preferably is not more than 20, more preferably not more than ten, still more preferably not more than five. The linear structure may be acyclic or cyclic. In the latter case, we have, e.g., 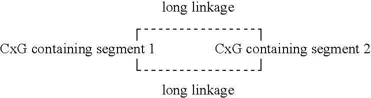
The cyclic multisegmented oligonucleotide may comprise more than two segments, with suitable linkages. Of course, even a single segment oligonucleotide of the present invention can be cyclic, e.g.,

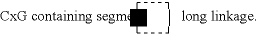

An advantage of a cyclic molecule is that it can form a stable three dimensional structure, which can enhance receptor binding affinity and specificity.

Alternatively, it may be branched, e.g., three segments each linked to a trivalent core element serving as the long internucleoside linkage for each of the three pairs of segments. Or four segments may be linked to a tetravalent core element. More complex branching structures are also possible, with a plurality of branching points.

A branched long linkage of particular interest is one comprising a pentaerythritol (PET) structure; such structures are discussed in Jiang et al., U.S. Provisional Appl. No. 60/378,645, filed May 9, 2002 (Docket: JIANG5-USA), incorporated by reference in its entirety.

The long linkages can, independently, be flexible or rigid. The length of the long linkage can be controlled so that the spacing of the CxG dinucleotide units substantially matches the spacing of receptor binding moieties.

Within a segment, if all of the international internucleoside linkages are chemically identical, we have a simple segment. If the linkages vary, we have a hybrid segment. GpApCpGpTpT would be a simple segment. GpApC x GpTpT, where the x denotes a short linkage other than phosphate (p), would be a hybrid segment. Any of the segments may be simple segments or hybrid segments.

Free Ends; 5′ and 3′ Ends

When, in this disclosure, it is stated that a lipophilic group is attached to a free end, or more particularly to the 5′ or 3′ end, it will be understood that this group is covalently incorporated into the end, that is, that it is at least a part of the end.

A linear oligonucleotide will have two free ends. In DNA and RNA, these free ends are identified as the 5′ and 3′ ends. In DNA and RNA, the nucleotides may be said to be connected by 5′ to 3′ phosphodiester linkages (C—O—P(═O)—O—C), which employ said phosphate groups. More particularly, the 5′ carbon of the sugar of one nucleotide is bonded to an oxygen of its phosphate group. Another oxygen of the same phosphate group is in turn bonded to the 3′ carbon of the sugar of the previous nucleotide.

It follows that the first nucleotide of DNA or RNA has a free phosphate group attached to the 5′ carbon. Likewise, because of the chemistry of the corresponding mononucleotide, the last nucleotide has a free hydroxyl group attached to its 3′ carbon. Hence, DNA and RNA normally have a 5′ phosphate and 3′ hydroxyl ends.

In the molecular biology art, it is known that one may modify the 5′ and/or 3′ ends of DNA or RNA. The most common modification is the conversion of the 3′ hydroxyl to a 31 phosphate. Likewise, the 5′ phosphate can be converted to a 5′ hydroxyl.

The terms 5′ and 3′ ends are readily applied, not only to DNA or RNA, but also to an analogue which retains the sugar unit of DNA or RNA and merely changes the internucleoside linkage without changing the points of attachment (the 5′ and 3′ carbons) on the sugar units. These terms may also be used for oligonucleotides with hybrid backbones, as long as the lipophilic group is attached to a DNA or RNA mononucleotide (see FIG. 6; attachment to 3′ end).

Reference to the 5′ end of an oligonucleotide shall, if the oligonucleotide does not in fact have a true 5′ or 3′ end because of its use of an alternative backbone, be defined as the end closest to the 5′ end of the internal DNA or RNA sequence, if any. If there is no DNA or RNA sequence whatsoever, the 5′ end shall be deemed to mean the end of the oligonucleotide which is closest to the cytosine (C) of at least one CxG dinucleotide unit. The 3′ end will be analogously defined by reference to the 3′ end of the internal DNA or RNA or, if need be, by reference to the location of the guanine of the CxG dinucleotide unit.

In a cyclic oligonucleotide, there are no free ends. However, lipophilic groups may be incorporated into the internucleoside linkages.

In a branched oligonucleotide, the branches have one free end, and one end that is attached, directly or indirectly, to the remainder of the oligonucleotide.

Lipophilic and Strongly Lipophilic Groups

Oligonucleotides, in general, are strongly hydrophilic (and lipophobic) by virtue of their phosphate groups. The standard nitrogenous bases adenine, guanine, cytosine, thymine and uracil are also hydrophilic.

The lipophilicity of the oligonucleotides of the present invention is increased by covalently incorporating into them one or more lipophilic groups, which more preferably are strongly lipophilic or highly lipophilic as defined below.

These lipophilic groups may be incorporated at one or more of the following sites:

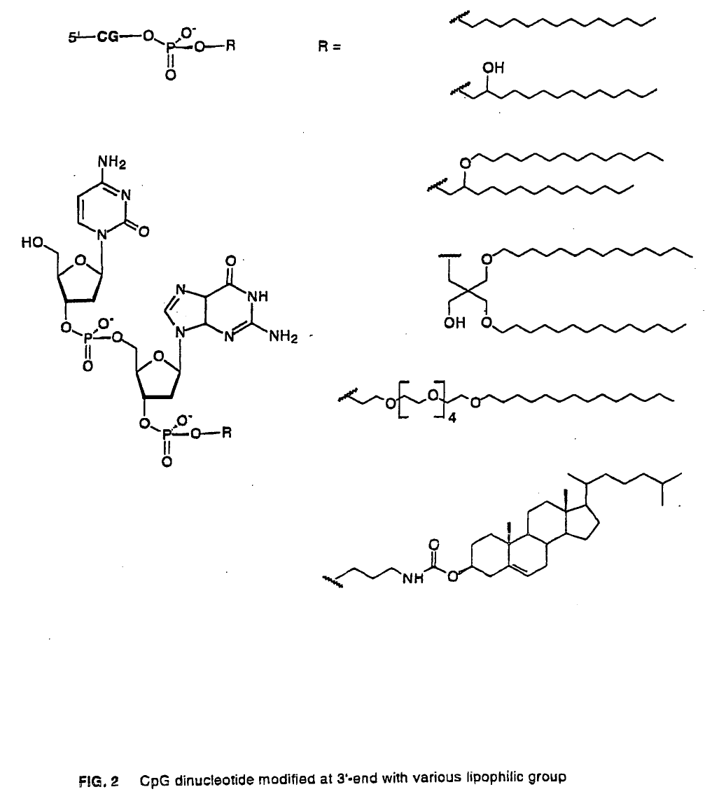

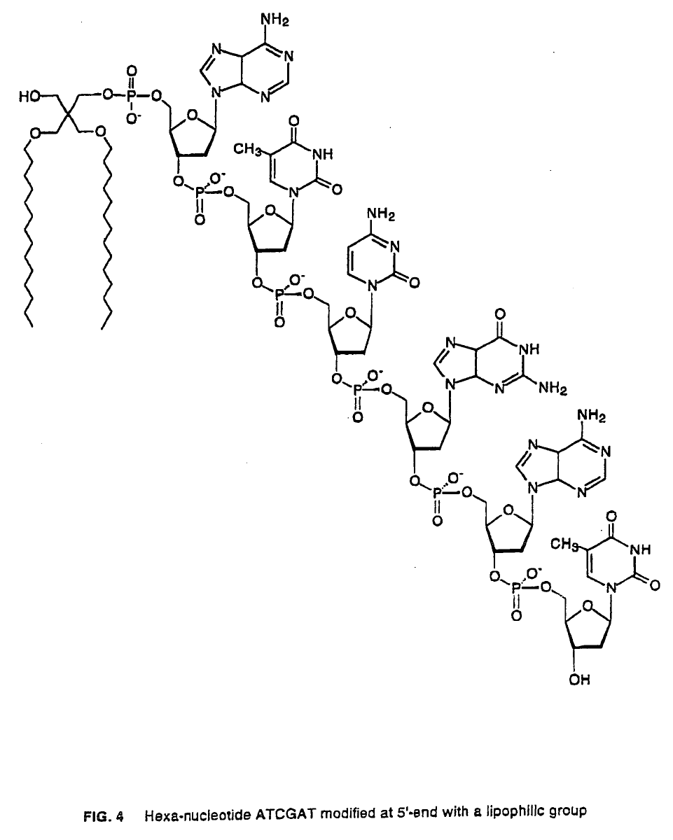

as at least part of one or both of the free ends of the molecule (see FIG. 2 for 3′ modification and FIG. 4 for 5′ modification);

as at least part of a substituent of a nucleoside; or

as at least part of an internucleoside linkage (see FIG. 3).

The oligonucleotide is deemed to comprise a lipophilic group if any moiety consisting of at least 5 atoms other than hydrogen qualifies as a lipophilic group by the criteria set forth below. Thus, the lipophilic group may be, e.g., a side chain on a terminal moiety or on the internucleoside linkage, as opposed to the entire terminal moiety or internucleoside linkage.

The incorporation may be direct or indirect, e.g., the lipophilic group may be attached to the 5′ or 3′ end through a phosphate group or an —O— linkage (the residue of a hydroxyl group), or some other linker.

When the 5′ or 3′ end of the oligonucleotide comprises a lipophilic group, preferably the entire 5′ or 3′ end, exclusive of any phosphate group, or of any analogue of a phosphate group in which one or more oxygens are replaced by sulfur, selenium or tellurium atoms, is lipophilic. Thus, in FIG. 7, compound 1, the lipophilic group has SMILES notation CC(O)CCCCCCCCCC.

FIG. 3 show lipophilic groups attached to a CpG dinucleotide with a standard sugar-phosphate backbone. The 3′ ends are modified to have a phosphate group which in turn is connected to the lipophilic group through one of the oxygens. FIG. 4 shows an oligonucleotide wherein the lipophilic group is attached to the 5′ phosphate end.

The lipophilic moieties may be aliphatic or aromatic, and linear or branched. Some of the preferred lipophilic moieties are depicted in FIG. 2, and may be characterized as follows:

linear aliphatic, 14 carbons

as above, but hydroxylated at C-2

branched aliphatic, one branch is 14 carbons, branching is at C-2 and goes through —O— to another 14 carbon chain.

branched aliphatic of form CH₂—C(CH₂OH) (CH₂—O-14 carbon alkyl)₂

linear aliphatic of form (CH₂CH₂O)₆-14 carbon alkyl

mixed aliphatic-fused aromatic (see FIG. 2).

See also FIG. 7, depicting the following preferred lipophilic groups:

1) —CH2(OH)-10 carbon alkyl

2) —PO4—(CH2CH2O)6-PO4-CH2OH-10 carbon alkyl

3) to 5) —PO4—CH₂OH-10 carbon alkyl

6) as above but 14 carbon alkyl

7) —CH2-O-14 carbon alkyl

The lipophilicity of a group may be determined by measuring the partition coefficient of the molecule HZ (where Z is the group in question) between a nonpolar solvent (e.g., ethanol, dioxane, acetone, benzene, n-octanol) and water, at STP. The lipophilicity may be defined as the logarithm of this partition coefficient; it will then be positive for molecules which prefer the nonpolar solvent. Thus, a lipophilic group is one for which logP is greater than zero.

The partition coefficient (P) is defined as the ratio of the equilibrium concentrations of a dissolved substance in a two-phase system consisting of two largely immiscible solvents. One such system is n-octanol:water; the octanol phase will contain about 20% water and the water phase about 0.008% octanol. Thus, the relevant partition coefficient (Pow) is the ratio of the molar concentration of the solute in octanol (O) saturated with water (w) to its molar concentration in water saturated with octanol. N-octanol is a useful surrogate for biological membranes because it, like many membrane components, is amphiphilic. (Reference hereafter to log P shall mean log Pow, unless otherwise stated.)

At least one lipophilic group is preferably a strongly lipophilic group. For the purpose of this disclosure, a strongly lipophilic group is defined as being one for which the lipophilicity calculated as the log of the n-octanol:water partition coefficient, by any of the three art-recognized methods set forth below as (A)-(C) is greater than that calculated for any of the side chains of the genetically encoded amino acids (hereafter, the reference side chains). The genetically encoded amino acids with lipophilic side chains are the aliphatic amino acids alanine, valine, leucine, isoleucine, and methionine, and the aromatic amino acids tryptophan, tyrosine and phenylalanine. (The rationale for using the lipophilic genetically encoded amino acids as a standard for “strongly lipophilic” is that they are part of PNAs.)

In one embodiment, the side chain in question is more lipophilic than any of the reference side chains when its lipophilicity, and that of the reference side chains, is determined according to method (A) below.

In a second embodiment, the side chain in question is more lipophilic than any of the reference side chains when its lipophilicity, and that of the reference side chains, is determined according to method (B) below.

In a third embodiment, the side chain in question is more lipophilic than any of the reference side chains when its lipophilicity, and that of the reference side chains, is determined according to method (C) below.

In a fourth embodiment, the side chain in question is more lipophilic than any of the reference side chains when its lipophilicity, and that of the reference side chains, are determined in accordance with a preferred method of determining the partition coefficient, which method is chosen on the basis of the predicted log Pow value (this predicted value is itself determined by method (C) below:

(A) for predicted log Pow values of 0 to 4, the shake flask method set forth in EPA Product Properties Test Guidelines OPPTS 830.7550 EPA 712-c-96-038 (August 1996)(Note that negative log Pow values imply that the compound is not lipophilic at all.)

(B) for predicted log Pow values of 4 to 6, the liquid chromatography estimation method set forth in the EPA Product Properties Test Guidelines OPP13 830.7570, EPA 712-C-96-040 (August 1996). (This method may be used for estimating Pow values of 0 to 6.)

(C) for predicted log Pow values higher than 6, the predictive method described in Meylan, et al., Atom/fragment contribution method for estimating octanol-water partition coefficients”, J. Pharm. Sci., 84: 83-92 (1995). (note that if predicted log Pow values are higher than 6, so experimental determination is necessary).

In Meylan's method, the predicted log Pow is obtained by adding weighted coefficients for each fragment (the raw coefficient multiplied by the number of copies of that fragment) to the constant 0.2290. The fragments considered include —CH3 (0.5473), —CH2- (0.4911), —CH (0.3614), —OH (−1.4086), —NH2 (−1.4148), —C(═O)N (−0.5236), —SH (−0.0001), —NH— (−1.4962), —N═C (−0.0010), —O— (−1.2566), ALDEHYDE-cho (−0.9422), -tert C so 3+C attached (0.2676), C no H not tert (0.9723), aromatic C (0.2940), aromatic N (5 membered ring) (−0.5262), and aromatically attached —OH (−0.4802); all aliphatic or aliphatically attached unless otherwise stated.

For more information on methods of determining Pow, see Sangster, J., Octanol-Water Partition Coefficients: Fundamentals and Physical Chemistry (April 1997) (ISBN 0-471-9739).

For tabulations of octanol-water partition coefficients, see the EPA “Chemicals in the Environment: OPPT Chemicals Fact Sheets” the USDA Pesticide Properties Database, Sangster, J., “Octanol-Water Partition Coefficients of Simple Organic Compounds”, J. Phys. Chem. Ref. Data, 18:1111-1230 (1989); Verbruggen, E. M. J., et al., “Physiochemical Properties of Higher Nonaromatic Hydrocarbons: Literature Study,” J. Phys. Chem. Ref. Data, 29:1435-46 (2000). For more sources, see references cited at Penn State University Libraries, Physical Sciences Library, octanol-water Partition Coefficients (last updated Aug. 21, 2001), at the URL libraries.psu.edu/crsweb/physci/coefficients.htm. It should be noted that the Pow values compiled for different compounds may have been determined by different methodologies.

The Meylan algorithm is implemented in the program LogPow (KowWin). An online version of the program, available at esc.syrres.com/interkow/kowdemo.htm, accepts either CAS registry numbers or SMILES structure notations. The program also reports experimentally determined values, if in its database.

A group is expected to be a lipophilic group if its logP, as predicted by the Meylan algorithm, is greater than zero. Preferably, the logP predicted by the Meylan algorithm is at least 1, at least 2, at least 3, at least 4, at least 4, at least 6, at least 7, at least 8, at least 9, or at least 10, the higher the more preferred.

At least one lipophilic group is preferably a “highly lipophilic (Meylan) group”. For the purpose of this disclosure, a “highly lipophilic (Meylan) group” is defined as one for which the lipophilicity calculated by the Meylan algorithm is at least 2.7. The highest logP predicted by the Meylan algorithm for the side chains of the genetically encoded amino acids is 2.60 (Trp); the highest experimentally determined logP for the same side chains is 2.89 (Ile). It should be noted that most “strongly lipophilic groups” will also be “highly lipophilic (Meylan) groups,” and vice versa.

Preferably, the lipophilic group will comprise not more than 100 atoms other than hydrogen, more preferably, not more than 80 such atoms, still more preferably, not more than 60 such atoms, even more preferably not more than 40 such atoms.

Preferably, the lipophilic group will comprise at least five atoms other than hydrogen (leucine has four such atoms), more preferably at least 11 such atoms (tryptophan has 10), still more preferably at least 13 such atoms, even more preferably at least 21 such atoms.

Preferably, the side chain has an elemental composition limited to the elements carbon, silicon, hydrogen, oxygen, nitrogen, sulfur, and phosphorous. Preferably, the majority of the bonds within the side chain which do not involve hydrogen are carbon-carbon bonds.

Preferably, the side chain is of the general form —Y-Z where —Y— is a spacer, and -Z is one or more aliphatic, and/or one or more aromatic moieties. The spacer is preferably selected from the group consisting of —O—, —S—, —NH—, —NR—, —PO4-, —C(═O)— and —C(═S)—. Z is preferably aliphatic. Alternatively, the spacer may be Y′, where Y′ is -alkyl-Y, and alkyl is a small alkyl of 1-4 carbon atoms. Other spacers, and other general forms, are permitted.

The aliphatic moieties, such as those of -Z, may, independently, comprise one or more spacers, which preferably are selected from the group defined above.

The lipophilic side chain may be entirely an aliphatic moiety or moieties, entirely an aromatic moiety or moieties, or a combination of at least one aliphatic moiety and at least one aromatic moiety.

Each aliphatic moiety may, independently, be linear, cyclic, a combination of linear and cyclic, branched but acyclic, or branched but with one or more branches comprising a cyclic moiety. It also may be saturated or unsaturated. If saturated, there may be one or more double and/or one or more triple bonds.

In one preferred embodiment, the side chain is a linear side chain which is an ether, i.e., —(CH2)i-O—(CH2)ⱼ, where “i” is 0 or 1 and j is 6 to 26. In compound 1a, “i” is 0 and “j” is 14.

In another preferred embodiment, the side chain is a two-branched aliphatic moiety, of the general structure —Y1Y2(Z1,Z2), where Y1 is null (i.e., the main chain carbon is directly bonded to Y2), or a spacer as defined above, Y2 is a small branched alkyl group connecting Y1 (or if Y1 is null, the main chain carbon) to Z1 and Z2, Z1 is —O(CH2)mCH3, and Z2 is —O(CH2)nCH3, where m and n are independently chosen integers in the range of 6-26. Preferably, Y1 is —C(═O)— or —NH—, and Y2 is —CH(CH2—)CH2-.

In another preferred embodiment, the side chain is a three branched aliphatic moiety, of the general structure —Y1Y2(Z1,Z2,Z3), where Y1 is null (i.e., the main chain carbon is directly bonded to Y2), or a spacer as defined above, Y2 is a small branched alkyl group connecting Y1 (or if Y1 is null, the main chain carbon) to Z1, Z2 and Z3, Z1 is —O(CH2)mCH3, Z2 is —O(CH2)nCH3, Z3 is —O(CH2)kCH3, and m, n and k are independently chosen from the range of 6-26. Y2 is preferably —C(CH2—)(CH2—)CH2-.

In another preferred embodiment, the side chain comprises one or more fatty acid moieties. Thus, the side chain may be of the form —Y1Y2(Z1 . . . Zi), where i is 1 to 2, Y1 is a previously defined, Y2 is null or is an alkyl group connecting the Y2 to the 1-3 Z groups, and at least one Z group is a fatty acid group of the form —O—CO-Q, where Q is primarily alkyl but may include alkenyl, alkynyl, or ether linkages. The fatty acids are carboxylic acids, often derived from or contained in an animal or vegetable fat or oil. All fatty acids are composed of a chain of hydrocarbon groups containing from 4 to 22 carbon atoms and characterized by a terminal carboxyl radical. They may be designated by “the number of carbon atoms: number of double bonds”, and optionally the locations of cis/trans isomerism. Thus, suitable fatty acids include those with designations 4:0, 6:0, 8:0, 10:0, 12:0, 14:0, 16:0, 16:1 (9c), 18:0, 18:1 (9c), 18:2 (9c, 12c), 18:3 (9c, 12c, 15c), 18:4 (6c, 9c, 12c, 15c), 18:3 (9c, 11t, 13t), 18:1 (9c) 12-OH, 20:1 (9c), 20:1 (11c), 20:4 (8c, 11c, 14c, 17c), 20:5 (5c, 8c, 11c, 14c, 17c), 22:0, 22:1 (11c), 22:1 (13c), 22:5 (7c, 10c, 13c, 16c, 19c) and 22:6 (4c, 7c, 10c, 13c, 16c, 19c), all of which are found in naturally occurring glycosides.

If the side chain comprises a plurality of cyclic moieties, they may be fused (forming a polycyclic moiety) or unfused, and may have the same or a different number of sides. Typically, they will each have 3-6 sides. One or more of the sides may be a double or triple bond. The cyclic moieties may be heterocyclic in character. In one preferred embodiment, the side chain comprises a steroid moiety. This is a polycyclic moiety with four fused rings, one five-sided and three six-sided.

The aliphatic moieties may comprise one or more phosphoryl groups, and, if they do so, the number of such groups in the side chain is preferably not more than two. Phosphoryl groups are found in the lipids of bacterial membranes, e.g., the lipid monophosphoryl lipid A (MPLA).

The aromatic moieties may comprise one or more rings. If there is more than one ring, the rings may be fused or unfused.

Using the program LogKow, we have calculated (see below) logP values for several preferred groups (Table K-1), and for some reference compounds (Table K-2). Compounds in Table K-2 are not necessarily inappropriate. Where LogKow also provided an experimental database value, this is also given below. The greyed rows in Table K-2 are for hydrophilic compounds.

TABLE K-1
Predicted and Experimental LogP Values of
Certain Preferred Lipophilic Groups

| SMILES (lower case | | LogP | |
|---|---|---|---|
| is arom) or CAS Reg. # | Comments | Pred | Exp |
| COCCCCC CCCCC | —C—O— linked 15C | 6.45 | ? |
| CCCCC | | arm | |
| MW 228.42 | | | |
| COCCCCC CCCCC | —C—O— linked 17C | 7.43 | ? |
| CCCCC CC | arm | | |
| COCCCCC CCCCC | —C—O— linked 13C | 5.47 | ? |
| CCC | arm | | |
| CO CCCCC CC | —C—O— linked 7C | 3.01 | ? |
| | arm | | |
| CO CCCCC CCCCC | —C—O— linked 27C | 12.84 | ? |
| CCCCC CCCCC | arm | | |
| CCCCC CC | | | |
| MW 410.77 | | | |
| CCO CCCCC CCCCC | —CCO-linked 15C | 7.43 | ? |
| CCCCC | arm | | |
| O═CC (COCCCCCCC) COCCCCCCC | 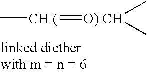 | 5.11 | ? |
| —MW 524.92 | 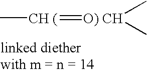 | 12.96 | ? |
| —MW 861.57 | 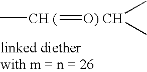 | 24.75 | ? |
| NC (COCCCCCCC) COCCCCCCC | 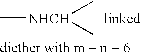 | 5.23 | ? |
| — | 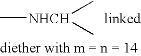 | 13.09 | ? |
| —MW 848.57 | 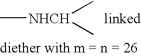 | 24.88 | ? |
| C (COCCCCCCC) COCCCCCCC | 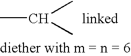 | 6.18 | ? |
| — | 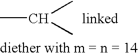 | 14.03 | ? |
| —MW 833.56 | 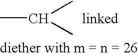 | 25.82 | ? |
| — | triether m = n = k = 6 | 8.78 | ? |
| CC (O) | Compounds 1, 3, | 4.7 | ? |
| CCCCC CCCCC | 4 and 5, FIG. 7, | | |
| | 3′ end | | |
| CCOCCOCCOCCOCCOCC | Compound 2, FIG. | 2.33 | ? |
| OP(═O) (O)O CC(O) | 7, 3′ end | | |
| CCCCC CCCCC | | | |
| CCCCC CCCCC CCCC | Compounds 1 and | 7.22 | 7.20 |
| | 6, FIG. 7, 3′ | | |
| | end | | |
| CC(O) | R of 2^{nd} compound | 5.68 | |
| CCCCC CCCCC | in FIG. 2 | | |
| CC | | | |
| CC | R of 3^{rd} compound | 12.76 | |
| (OCCCCCCCCCCCCCC) | in FIG. 2 | | |
| CCCCCCCCCCCC | | | |
| CC(CO) | R of 4^{th} compound | 12.46 | |
| (COCCCCCCCCCCCCCC) | in FIG. 2 | | |
| COCCCCCCCCCCCCCC | | | |
| CCOCCOCCOCCOCCOCC | R of 5^{th} compound | 5.57 | |
| O | in FIG. 2 | | |
| CCCCC CCCCC | | | |
| CCCC | | | |
| CCCNC(═O) | R of 6^{th} compound | 10.31 | |
| OC(CCC(C1═CCC2C(C | in FIG. 2 | | |
| (C(C3)C(CCCC(C)C) | (cholesterol- | | |
| C) (CC4)C)C3) (C24) | related, note | | |
| C)C1 | boldfaced SMILES) | | |

TABLE K-2
Predicted and Experimental LogP Values for Certain Reference Compounds

| | SMILES | | | |
|---|---|---|---|---|
| | (lower case | | | |
| | is arom) or | | LogP | |
| Compound | CAS Reg. # | Comments | Pred | Exp |
| 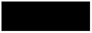 | 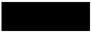 | 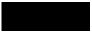 | 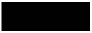 | 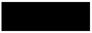 |
| 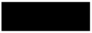 | 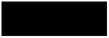 | 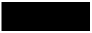 | 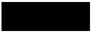 | 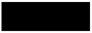 |
| 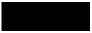 | 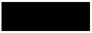 | 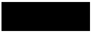 | 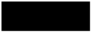 | 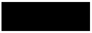 |
| 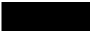 | 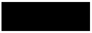 | 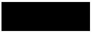 | 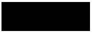 | 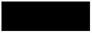 |
| 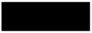 | 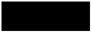 | 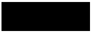 | 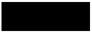 | 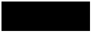 |
| 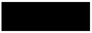 | 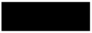 | 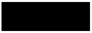 | 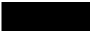 | 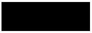 |
| 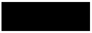 | 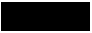 | 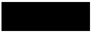 | 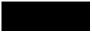 | 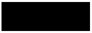 |
| 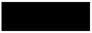 | 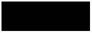 | 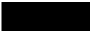 | 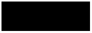 | 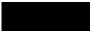 |
| 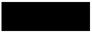 | 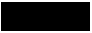 | 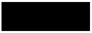 | 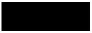 | 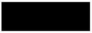 |
| 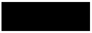 | 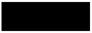 | 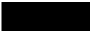 | 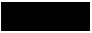 | 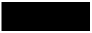 |
| 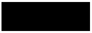 | 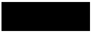 | 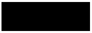 | 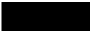 | 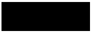 |
| 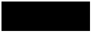 | 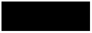 | 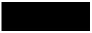 | 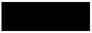 | 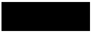 |
| psoralen | 66-97-7 | | 2.06 | 1.67 |
| acridine | 260-94-6 | | 3.32 | 3.40 |
| biotin | 58-85-5 | | 0.39 | |
| cholesterol | 57-88-5 | | 8.74 | |
| methane | C | Ala side c | 0.78 | 1.09 |
| Propane | C(C)C | Val side c | 1.81 | 2.36 |
| Ethane, | CCSC | Met side c | 1.41 | 1.54 |
| (methylthio)- | | | | |
| n-butane | CCCC | Ile side | 2.31 | 2.89 |
| | | chain | | |
| 2-methyl | CC(C)C | Leu side c | 2.23 | 2.76 |
| propane | | | | |
| toluene | Cclcccccl | Phe side c | 2.54 | 2.73 |
| p-cresol | Cclccc(O)ccl | Tyr side c | 2.06 | 1.94 |
| 3-methyl | Cclcnc2ccccc | Trp side c | 2.60 | 2.60 |
| indole | 12 | | | |
| methyl | CO CCCC | | 1.54 | 1.66 |
| n-butyl | | | | |
| ether | | | | |
| n-pentane | CCCCC | one more c | 2.80 | 3.39 |
| | | than Leu | | |
| | OP(═O)(O)O | Compounds | 3.15 | ? |
| | CC(O) | 1, FIG. 7, | | |
| | CCCCC CCCCC | 3′ end | | |
| | | incl. | | |
| | | Phosphate | | |
| | OP(═O)(O)O | Compound | 0.78 | ? |
| | CCOCCOCCOCCO | 2, FIG. 7, | | |
| | CCOCC | 3′ end | | |
| | OP(═O)(O)O | incl. both | | |
| | CC(O) | phosphates | | |
| | CCCCC CCCCC | | | |
| | OP(═O)(O)O | Compound | 5.67 | ? |
| | CCCCC CCCCC | 6, FIG. 7, | | |
| | CCCC | 3′ end, | | |
| | | including | | |
| | | phosphate | | |

Targeting Moieties

The oligonucleotides of the present invention may additionally comprise one or more cell-type specific targeting moieties to improve delivery to a particular cell type. For example, they may comprise a carbohydrate or peptide moiety which is specifically recognized by a cell surface receptor. Useful carbohydrate moieties include galactose, N-acetylgalactosamine (GalNAc), fucose, mannose, and sialic acid (N-acetyl neuraminic acid).

Other Immunomodulatory Moieties

The oligonucleotides of the present invention may further comprise one or more immunomodulatory moieties other than the CxG dinucleotide unit.

For example, they may comprise a thymidine dimer, as previously noted. They may also comprise lipoteichoic acid or a derivative thereof. LTA is a membrane component of gram positive bacteria and can activate the innate immune response. One possible derivative is one in which the ester linkage between D-Alanine and the secondary hydroxyl group of the glycerol moiety is replaced by an amide bond, which will increase resistance to hydrolysis. The LTA-like moiety may be attached to the 3′ or 5′ ends of the oligonucleotide, or incorporated into one or more internucleoside linkages.

Immunogen

The immunogen of the present invention is a molecule comprising at least one disease-associated B or T cell epitope, as defined below, and which, when suitably administered to a subject (which, in some cases, may mean associated with a liposome or with an antigen-presenting cell), elicits a humoral and/or cellular immune response which is protective against the disease.

The immunostimulatory oligonucleotide of the present invention may be administered with the immunogen (in the same or a separate composition), or before or after administration of the immunogen, provided that the interval between the administration of the oligonucleotide and the immunogen is not so long that the oligonucleotide cannot potentiate the immune response to the immunogen.

The most preferred immunogenic composition comprises the BLP25 liposomal vaccine formulation described in Koganty et al., Synthetic Glyco-Lipo-Peptides as Vaccines, U.S. Provisional Appl. No. 60/377,595, filed May 6, 2002 (DOcket: Koganty4.1-USA), which is hereby incorporated by reference in its entirety. This liposomal formulation comprises a MUC1-derived 25-mer lipopeptide, also described therein, which is the most preferred immunogen. The composition may include a oligonucleotide as an adjuvant. It may also include other adjuvants or other immunological agents (e.g., cytokines).

Epitope

The epitopes of the present invention may be B-cell or T-cell epitopes, and they may be of any chemical nature, including without limitation, peptides, carbohydrates, lipids, glycopeptides and glycolipids. The epitope is at least substantially the same as a naturally occurring epitope. It may be identical to a naturally occurring epitope, or a modified form of a naturally occurring epitope.

A term such as “MUC1 epitope”, without further qualification, is intended to encompass, not only a native epitope of MUC1, but also a mutant epitope which is substantially identical to a native epitope. Such a mutant epitope must be cross-reactive with a native MUC1 epitope. Likewise, a term such as “tumor-associated epitope” includes both native and mutant epitopes, but the mutant epitope must be cross-reactive with a native tumor-associated epitope.

B-Cell Epitopes

B-cell epitopes are epitopes recognized by B-cells and by antibodies.

B-cell peptide epitopes are typically at least five amino acids, more often at least six amino acids, still more often at least seven or eight amino acids in length, and may be continuous (“linear”) or discontinuous (“conformational”) (the latter being formed by the folding of a protein to bring noncontiguous parts of the primary amino acid sequence into physical proximity).

B-cell epitopes may also be carbohydrate epitopes.

T Cell Epitopes

The T cell epitope, if any, may be any T cell epitope which is at least substantially the same as a T-cell epitope of an antigen including a hapten) which is associated with a disease or adverse condition to a degree such that it could be prophylactically or therapeutically useful to stimulate or enhance a cellular immune response to that epitope. Such diseases and conditions include, but are not limited to parasitic diseases such as schistosomiasis and leishmania, fungal infections such as candidiasis, bacterial infections such as leprosy, viral infections such as HIV infections, and cancers, especially solid tumors. Of course, the greater the degree of specificity of the epitope for the associated disease or adverse condition, the more likely it is that the stimulation of an immune response to that epitope will be free of adverse effects.

The epitope must, of course, be one amenable to recognition by T-cell receptors so that a cellular immune response can occur. For peptides, the T-cell epitopes may interact with class I or class II MHC molecules. The class I epitopes usually 8 to 15, more often 9-11 amino acids in length. The class II epitopes are usually 5-24 (a 24 mer is the longest peptide which can fit in the Class II groove), more often 8-24 amino acids. If the immunogen is larger than these sizes, it will be processed by the immune system into fragments of a size more suitable for interaction with MHC class I or II molecules.

The carbohydrate T-cell epitopes may be as small as a single sugar unit (e.g., Tn). They are preferably no larger than five sugars.

Many T-cell epitopes are known. Several techniques of identifying additional T-cell epitopes are recognized by the art. In general, these involve preparing a molecule which potentially provides a T-cell epitope and characterizing the immune response to that molecule. Methods of characterizing the immune response are discussed in a later section.

The reference to a CTL epitope as being “restricted” by a particular allele of MHC Class I molecules, such as HLA-Al, indicates that such epitope is bound and presented by the allelic form in question. It does not mean that said epitope might not also be bound and presented by a different allelic form of MHC, such as HLA-A2, HLA-A3, HLA-B7, or HLA-B44.

Disease-Associated and Disease-Specific Epitopes

A disease is an adverse clinical condition caused by infection or parasitization by a virus, unicellular organism, or multicellular organism, or by the development or proliferation of cancer (tumor) cells. (The cancers of interest include those set forth in WO98/18810 and WO99/51259.)

The unicellular organism may be any unicellular pathogen or parasite, including a bacteria, fungus or protozoan. (The viruses, bacteria and fungi of interest include those set forth in WO98/18810 and WO99/51259.) The multicellular organism may be any pathogen or parasite, including a protozoan, worm, or arthropod. Multicellular organisms include both endoparasites and ectoparasites. Endoparasites are more likely to elicit an immune response, but, to the extent they can elicit a protective immune response, ectoparasites and their antigens are within the purview of the present invention.

An epitope may be said to be directly associated with a viral disease if it is presented by a virus particle, or if it is encoded by the viral genome and expressed in an infected cell.

An epitope may be said to be directly associated with a disease caused by a unicellular or multicellular organism if it presented by an intracellular, surface, or secreted antigen of the causative organism.

An epitope may be said to be directly associated with a particular tumor if it is presented by an intracellular, surface or secreted antigen of said tumor. It need not be presented by all cell lines of the tumor type in question, or by all cells of a particular tumor, or throughout the entire life of the tumor. It need not be specific to the tumor in question. An epitope may be said to be “tumor associated” in general if it is so associated with any tumor (cancer, neoplasm).

Tumors may be of mesenchymal or epithelial origin. Cancers include cancers of the colon, rectum, cervix, breast, lung, stomach, uterus, skin, mouth, tung, lips, larynx, kidney, bladder, prostate, brain, and blood cells.

An epitope may be indirectly associated with a disease if the epitope is of an antigen which is specifically produced or overproduced by infected cells of the subject, or which is specifically produced or overproduced by other cells of the subject in specific, but non-immunological, response to the disease, e.g., an angiogenic factor which is overexpressed by nearby cells as a result of regulatory substances secreted by a tumor.

The term “disease associated epitope” also includes any non-naturally occurring epitope which is sufficiently similar to an epitope naturally associated with the disease in question so that antibodies or T cells which recognize the natural disease epitope also recognize the similar non-natural epitope. Similar comments apply to epitopes associated with particular diseases or classes of diseases.

An epitope may be said to be specific to a particular source (such as a disease-causing organism, or, more particular, a tumor), if it is associated more frequently with that source than with other sources, to a detectable and clinically useful extent. Absolute specificity is not required, provided that a useful prophylactic, therapeutic or diagnostic effect is still obtained.

In the case of a “specific tumor-specific” epitope, the epitope is more frequently associated with that tumor that with other tumors, or with normal cells. Preferably, there should be a statistically significant (p=0.05) difference between its frequency of occurrence in association with the tumor in question, and its frequency of occurrence in association with (a) normal cells of the type from which the tumor is derived, and (b) at least one other type of tumor. An epitope may be said to be “tumor-specific” in general is it is associated more frequently with tumors (of any or all types) than with normal cells. It need not be associated with all tumors.

The term “tumor specific epitope” also includes any non-naturally occurring epitope which is sufficiently similar to a naturally occurring epitope specific to the tumor in question (or as appropriate, specific to tumors in general) so that antibodies or T cells stimulated by the similar epitope will be essentially as specific as CTLs stimulated by the natural epitope.

In general, tumor-versus-normal specificity is more important than tumor-versus-tumor specificity as (depending on the route of administration and the particular normal tissue affected), higher specificity generally leads to fewer adverse effects. Tumor-versus-tumor specificity is more important in diagnostic as opposed to therapeutic uses.

The term “specific” is not intended to connote absolute specificity, merely a clinically useful difference in probability of occurrence in association with a pathogen or tumor rather than in a matched normal subject.

Parasite-Associated Epitopes

In one embodiment, the epitope is a parasite-associated epitope, such as an epitope associated with leishmania, malaria, trypanosomiasis, babesiosis, or schistosomiasis. Suitable parasite-associated epitopes include, but are not limited to, the following.

| Parasite | Epitope | References |
|---|---|---|
| Plasmodium Falciparum | (NANP)3 | Good et al (1986) |
| (Malaria | | J. Exp. Med. 164: 655 |
| | Circumsporoz. | Good et al (1987 |
| | protein | Science 235: 1059 |
| | AA 326-343 | |
| Leishmania donovani | Repetitive | Liew et al (1990) |
| | peptide | J. Exp. Med. 172: 1359 |
| Leishmani major | EAEEAARLQA | Longenecker, |
| | (SEQ ID NO: 4) | et al., 08/229,606 |
| Toxoplasma gondii | P30 surface | Darcy et al (1992) |
| | protein | J. Immunolog. 149: 3636 |
| Schistosoma mansoni | Sm-28GST | Wolowxzuk et al (1991) |
| | antigen | J. Immunol 146: 1987 |

Virus-Associated Epitopes

In another embodiment, the epitope is a viral epitope, such as an epitope associated with human immunodeficiency virus (HIV), Epstein-Barr virus (EBV), or hepatitis. Suitable viral epitopes include, but are not limited to:

| Virus | Epitope | Reference |
|---|---|---|
| HIV gp120 | V3 loop, 308-331 | Jatsushita, S. et al (1988) |
| | | J. Viro. 62: 2107 |
| HIV GP120 | AA 428-443 | Ratner et al (1985) |
| | | Nature 313: 277 |
| HIV gp120 | AA 112-124 | Berzofsky et al (1988) |
| | | Nature 334: 706 |
| HIV | Reverse transcriptase | Hosmalin et al (1990) |
| | | PNAS USA 87: 2344 |
| Flu | nucleoprotein | Townsend et al (1986) |
| | AA 335-349, 366-379 | Cell 44: 959 |
| Flu | haemagglutinin | Mills et al (1986) |
| | AA48-66 | J. Exp. Med. 163: 1477 |
| Flu | AA111-120 | Hackett et al (1983) |
| | | J. Exp. Med 158: 294 |
| Flu | AA114-131 | Lamb, J. and Green N. (1983) |
| | | Immunology 50: 659 |
| Epstein-Barr | LMP43-53 | Thorley-Lawson et al (1987) |
| | | PNAS USA 84: 5384 |
| Hepatitis B | Surface Ag AA95-109; | Milich et al (1985) |
| | AA 140-154 | J. Immunol. 134: 4203 |
| | Pre-S antigen | Milich, et al. (1986) |
| | AA 120-132 | J. Exp. Med. 164: 532 |
| Herpes simplex | gD protein AA5-23 | Jayaraman et al (1993) |
| | | J. Immunol. 151: 5777 |
| | gD protein AA241-260 | Wyckoff et al (1988) |
| | | Immunobiology 177: 134 |
| Rabies | glycoprotein AA32-44 | MacFarlan et al (1984) |
| | | J. Immunol 133: 2748 |

Bacteria-Associated Epitopes

The epitope may also be associated with a bacterial antigen. Suitable epitopes include, but are not limited to:

| Bacteria | Epitope ID | Reference |
|---|---|---|
| Tuberculosis | 65 Kd protein | Lamb et al (1987) |
| | AA112-126 | EMBO J. 6: 1245 |
| | AA163-184 | |
| | AA227-243 | |
| | AA242-266 | |
| | AA437-459 | |
| Staphylococcus | nuclease protein | Finnegan et al (1986) |
| | AA61-80 | J. Exp. Med 164: 897 |
| E. coli | heat stable enterotoxin | Cardenas et al (1993) |
| | | Infect Immunity 61: 4629 |
| | heat labile entertoxin | Clements et al (1986) |
| | | Infect. Immunity 53: 685 |
| Shigella sonnei | form I antigen | Formal et al (1981) |
| | | Infect. Immunity 34: 746 |

Cancer-Associated Epitopes

In another embodiment, the epitope is associated with a cancer (tumor), including but not limited to cancers of the respiratory system (lung, trachea, larynx), digestive system (mouth, throat, stomach, intestines) excretory system (kidney, bladder, colon, rectum), nervous system (brain), reproductive system (ovary, uterus, cervix), glandular system (breast, liver, pancreas, prostate), skin, etc. The two main groups of cancers are sarcomas, which are of mesenchymal origin and affect such tissues as bones end muscles, and carcinomas, which are of epithelial origin and make up the great majority of the glandular cancers of breasts, stomach, uterus, skin and tongue. The sarcomas include fibrosarcomas, lymphosarcomas, osteosarcomas, chondrosarcomas, rhabdosarcomas and liposarcomas. The carcinomas include adenocarcinomas, basal cell carcinomas and squamous carcinomas.

Cancer-associated epitopes include, but are not limited to, peptide epitopes such as those of mutant p53, the point mutated Ras oncogene gene product, her 2/neu, c/erb2, and the MUC1 core protein, and carbohydrate epitopes such as sialyl Tn (STn), TF, Tn, CA 125, sialyl Le^{x}, sialyl Le^{a} and P97.

Carbohydrate Epitopes

Carbohydrate epitopes are also of interest. For example, any of three types of tumor-associated carbohydrate epitopes which are highly expressed in common human cancers may be presented. These particularly include the lacto series type 1 and type 2 chains, cancer associated ganglio chains, and neutral glycosphingolipids. Examples of the lacto series Type 1 and Type 2 chains are as follows: Lewis a, dimeric Lewis a, Lewis b, Lewis b/Lewis a, Lewis x, Lewis, y, Lewis a/Lewis x. dimeric Lewis x, Lewis y/Lewis x, trifucosyl Lewis y, trifucosyl Lewis b, sialosyl Lewis x, sialosyl Lewis y, sialosyl dimeric Lewis x, Tn, sialosyl Tn, sialosyl TF, TF. Examples of cancer-associated ganglio chains are as follows: GM3. GD3, GM2, GM4, GD2, GM1, GD-1a, GD-1b. Neutral sphingolipids include globotriose, globotetraose, globopentaose, isoglobotriose, isoglobotetraose, mucotriose, mucotetraose, lactotriose, lactotetraose, neolactotetraose, gangliotriose, gangliotetraose, galabiose, and 9-O-acetyl-GD3.

Numerous antigens of clinical significance bear carbohydrate determinants. One group of such antigens comprises the tumor-associated mucins (Roussel, et al., Biochimie 70, 1471, 1988).

Generally, mucins are glycoproteins found in saliva, gastric juices, etc., that form viscous solutions and act as lubricants or protectants on external and internal surfaces of the body. Mucins are typically of high molecular weight (often >1,000,000 Dalton) and extensively glycosylated. The glycan chains of mucins are O-linked (to serine or threonine residues) and may amount to more than 80% of the molecular mass of the glycoprotein. Mucins are produced by ductal epithelial cells and by tumors of the same origin, and may be secreted, or cell-bound as integral membrane proteins (Burchell, et al., Cancer Res., 47, 5476, 1987; Jerome, et al., Cancer Res., 51, 2908, 1991).

Cancerous tissues produce aberrant mucins which are known to be relatively less glycosylated than their normal counter parts (Hull, et al., Cancer Commun., 1, 261, 1989). Due to functional alterations of the protein glycosylation machinery in cancer cells, tumor-associated mucins typically contain short, incomplete glycans. Thus, while the normal mucin associated with human milk fat globules consists primarily of the tetrasaccharide glycan, gal β1-4 glcNAcpl-6(gal β1-3) gal NAc-α and its sialylated analogs (Hull, et al.), the tumor-associated Tn hapten consists only of the monosaccharide residue, α-2-acetamido-3-deoxy-D-galactopyranosyl, and the T-hapten of the disaccharide β-D-galactopyranosyl-(1-3)α-acetamido-2-deoxy-D-galactopyranosyl. Other haptens of tumor-associated mucins, such as the sialyl-Tn and the sialyl-(2-6)T haptens, arise from the attachment of terminal sialyl residues to the short Tn and T glycans (Hanisch, et al., Biol. Chem. Hoppe-Sevler, 370, 21, 1989; Hakormori, Adv. Cancer Res., 52:257, 1989; Torben, et al., Int. J. Cancer, 45 666, 1980; Samuel, et al., Cancer Res., 50, 4801, 1990).

The T and Tn antigens (Springer, Science, 224, 1198, 1984) are found in immunoreactive form on the external surface membranes of most primary carcinoma cells and their metastases (>90% of all human carcinomas). As cancer markers, T and Tn permit early immunohistochemical detection and prognostication of the invasiveness of some carcinomas (Springer). Recently, the presence of the sialyl-Tn hapten on tumor tissue has been identified as an unfavorable prognostic parameter (Itzkowitz, et al. Cancer, 66, 1960, 1990; Yonezawa, et al., Am. J. Clin. Pathol., 98 167, 1992). Three different types of tumor-associated carbohydrate antigens are highly expressed in common human cancers. The T and Tn haptens are included in the lacto series type, and type 2 chains. Additionally, cancer-associated ganglio chains and glycosphingolipids are expressed on a variety of human cancers.

The altered glycan determinants displayed by the cancer associated mucins are recognized as non-self or foreign by the patient's immune system (Springer). Indeed, in most patients, a strong autoimmune response to the T hapten is observed. These responses can readily be measured, and they permit the detection of carcinomas with greater sensitivity and specificity, earlier than has previously been possible. Finally, the extent of expression of T and Tn often correlates with the degree of differentiation of carcinomas. (Springer).

An extensive discussion of carbohydrate haptens appears in Wong, U.S. Pat. No. 6,013,779. A variety of carbohydrates can be incorporated into a synthetic glycolipopeptide immunogen, according to the present invention, for use particularly in detecting and treating tumors. The Tn, T, sialyl Tn and sialyl (2——>6)T haptens are particularly preferred.

In particular, for detecting and treating tumors, the three types of tumor-associated carbohydrate epitopes which are highly expressed in common human cancers are conjugated to aminated compounds. These particularly include the lacto series type 1 and type 2 chain, cancer associated ganglio chains, and neutral glycosphingolipids.

Examples of the lacto series Type 1 and Type 2 chains are as follows:

Lacto Series Type A and Type 2 Chain

```
Lewis a:
          Fucα 1
                    ↓
                    4
Ga1β1→3G1cNAcβ1→
dimeric Lewis a:
          Fucα 1 Fucα 1
                    ↓ ↓
                    4 4
Ga1β1→3G1cNAcβ1→Ga1β1→3G1cNAcβ1→
Lewis b:
            Fucα 1
                      ↓
                      4
        Ga1β1→3G1cNAcβ1→
          2
          ↑
Fucα 1
Lewis b/Lewis a:
        Fucα 1 Fucα 1
                  ↓ ↓
                  4 4
  Ga1β1→3G1cNacβ1→Ga1β1→G1cNacβ1→
          2
          ↑
Fucα 1
Lewis x:
Ga1β1→4G1cNAcβ1→
                    3
                    ↑
          Fucα 1
Lewis y:
        Ga1β1→4G1cNAcβ1→
          2 3
          ↑ ↑
Fucα 1 Fucα 1
Lewis a/Lewis x:
Ga1β1→3G1cNAcβ1→3Ga1β1→4G1cNAcβ→
                                                  3
                                                  ↑
                                        Fucα 1
Lewis x/Lewis x (dimeric Le^{x}):
Ga1β1→4G1cNAcβ1→3Ga1β1→4G1cNAcβ
                    3 3
                    ↑ ↑
          Fucα 1 Fucα 1
Lewis y/Lewis x:
        Ga1β1→4G1cNAcβ1→3Ga1β1→4G1cNAcβ→
          2 3 3
          ↑ ↑ ↑
Fucα 1 Fucα 1 Fucα 1
Trifucosyl Lewis y:
        Ga1β1→4G1cNAcβ1→3Ga1β1→4G1cNAcβ1→3Ga1β1→4G1cβ1→
          2 3 3
          ↑ ↑ ↑
Fucα 1 Fucα 1 Fucα 1
Trifucosyl Lewis b:
                  Fucα 1
                            ↓
        Ga1β1→3G1cNAcβ1→3Ga1β1→4G1cNAcβ1→3Ga1β1→4G1cβ1→
          2 3
          ↑ ↑
Fucα 1 Fucα 1
Sialosyl Le^{x}:
NeuAcα2→3Ga1β1→4G1cNAcβ1→
                                    3
                                    ↑
                          Fucα 1
Sialosyl Le^{a}:
                          Fucα 1
                                    ↓
                                    4
NeuAcα2→3Ga1β1→3GlcNAcβ1→
Sialosyl Dimeric Le^{x}:
NeuAcα2→3Ga1β1→4G1cNAcβ1→3Ga1β1→4G1cNAcβ1→
                                    3 3
                                    ↑ ↑
                          Fucα 1 Fucα 1
Tn:
GalNAcα1→
Sialosyl-Tn:
NeuAcα→6Ga1NAcα1→
Sialosyl-T:
NeuAcα→6 (Ga1β1→3) Ga1NAcα1→
NeuAcα→6Ga1NAcα1→
                  3
                  ↓
        Ga1β 1
T:
Ga1β1→3Ga1NAcα1→
```

Examples of cancer-associated ganglio chains that can be conjugated to aminated compounds according to the present invention are as follows:

Cancer Associated Ganglio Chains

```
GM3:
NeuAcα2→3Ga1β1→4G1cβ1→
GD3:
NeuAcα2→8NeuAcα2→3Ga1β1→4G1cβ1→
GM2:
Ga1NAcβ1→4Ga1β1→4G1cβ1→
                          3
                          ↑
            NeuAcα 2
GM4:
NeuAcα2→3Galβ1→
GD2:
Ga1NAcβ1→4Ga1β1→4G1cβ1→
                                3
                                ↑
NeuAcα2→8NeuAcα 2
GM1:
Ga1β1→3Ga1NAcβ1→4Ga1β1→4G1cβ1→
                          3
                          ↑
            NeuAcα 2
GD-1a:
NeuAcα2→3Ga1β1→3Ga1NAcβ1→4Galβ1→4G1cβ1→
                                      3
                                      ↑
                        NeuAcα 2
GD-1b:
      Ga1β1→3Ga1NAcβ1→4Ga1β1→4G1cβ1→
                                3
                                ↑
NeuAcα2→8NeuAcα 2
```

In addition to the above, neutral glycosphingolipids can also be conjugated to aminated compounds according to the present invention:

Selected Neutral Glycosphingolipids

Globotriose: Galα→4Galβ1→4Glcβ1→

Globotetraose: GalNAcβ1→3Galα→4Galβ1→4Glcβ1→

Globopentaose: GalNAcα1→3GalNAcβ1→3Galα→4Galβ1→4Glcβ1→

Isoglobotriose: Galα→3Galβ1→4Glcβ1→

Isoglobotetraose: GalNAcβ1→3Galα1→3Galβ1→4Glcβ1→

Mucotriose: Galβ1→4Galβ1→4Glcβ1→

Mucotetraose: Galβ1→3Galβ1→4Galβ1→4Glcβ1→

Lactotriose: GalNAcβ1→3Galβ1→4Glcβ1→

Lactotetraose: GalNAcβ1→3GalNAcβ1→3Galβ1→4Glcβ1→

Neolactotetraose: Galβ1→4GlcNAcβ1→3Galβ1→4Glcβ1→

Gangliotriose: GalNAcβ1→4Galβ1→4Glcβ1→

Gangliotetraose: Galβ1→GlcNAcβ1→4Galβ1→4Glcβ1→

Galabiose: Galα→4Galβ1→

9-0-Acetyl-GD3: 9-0-Ac-NeuAcα2→8NeuAcα2→3Galβ1→4Glcβ1→

Clustered Epitopes

In some embodiments, more than one epitope is provided, and the epitopes are clustered.

Carbohydrate epitope clusters have been reported in the literature, but the significance of these have not yet been clearly defined. See Reddish, et al., Glycoconjugate J., 14:549-60 (1997) (clustered STn), Ragapathi, et al. Cancer Immunol. Immunother. 48:1-8 (1999). Likewise, clusters of O-glycosylation sites have been reported. See Gendler, et al., J. Biol. Chem., 263:12820 (1988).

The following U.S. patents use the phrase “clustered epitopes”:

U.S. Pat. No. 6,376,463

U.S. Pat. No. 6,258,937

U.S. Pat. No. 6,180,371

U.S. Pat. No. 5,929,220

U.S. Pat. No. 5,888,974

U.S. Pat. No. 5,859,204

U.S. Pat. No. 5,744,446

The following U.S. patents recited “clustered” and “carbohydrate epitopes”:

U.S. Pat. No. 6,365,124

U.S. Pat. No. 6,287,574

U.S. Pat. No. 6,013,779

U.S. Pat. No. 5,965,544

U.S. Pat. No. 5,268,364

U.S. Pat. No. 4,837,306

Mucin Epitope

In a preferred embodiment, the epitope is an epitope of a cancer-associated mucin. Mucins are glycoproteins characterized by high molecular weight (>1,000,000 daltons) and extensive glycosylation (over 80%). Mucins may be expressed extracellularly, or as an integral cell membrane glycoprotein with distinct external, transmembrane, and cytoplasmic domains. Cell membrane mucins exist as flexible rods and protrude relatively great distances from the cell surface forming an important component of the glycocalyx (Jentoff, 1990) and the terminal carbohydrate portions thereof are probably the first point of contact with antibodies and cells of the immune system.

Abberant or cancer-associated mucins are known to be relatively less glycosylated (Hull et al, 1989) and hence antigenically different from their normal cell counterpart mucins exposing normally cryptic carbohydrate—(Hanish et al, 1989; Torben et al, 1990; Samuel et al, 1990), peptide—(Burchell et al, 1987) and perhaps even glycopeptide-epitopes. Therefore, because cell surface mucins protrude, they themselves may serve as targets for immune attack (Henningson, et al., 1987; Fung, et al., 1990; Singhal, et al., 1991; Jerome et al., 1991; Oncogen, EP 268,279; Biomembrane Institute, WO89/08711; Longenecker, U.S. Pat. No. 4,971,795). Under some circumstances, cancer-associated cell membrane mucins can actually “mask” other cell surface antigens and protect cancer cells from immune attack (Codington et al, 1983; Friberg, 1972; Miller et al, 1977).

The mucin epitope may be a core peptide, a carbohydrate, or a glycopeptide. Non-limiting examples of mucins which may carry epitopes are the human tumor associated Thomsen-Friedenreich antigen, (MacLean, 1992), epiglycanin-related glycoprotein (Codington, 1984) ovine submaillary mucin, bovine submaxillary mucin, breast tumor mucins (e.g., human polymorphic epithelial mucin, including breast tumor mucins, Gendler, 1988, 1990; breast cancer epithelial tumor antigen, Hareuveni, 1990, breast carcinoma, Hull, 1989), mammary tumor mucins (e.g., such as murine mammary adenocarcinoma, Fung, 1990) carcinoma mucins such as mucins arising from the kidney (e.g., renal cell carcinoma), ovary (e.g., ovarian carcinoma-associated sebaceous gland antigen, Layton, 1990), bladder, colon (e.g., Sialosyl-Tn in colorectal cancer, Itzkowitz, 1990) pancreatic tumor mucin (Lan, 1990), gallbladder, bladder, colon (e.g., malignant colon mucosa mucins, Torbin, 1980) and some lung tissues, melanoma mucins (e.g., melanoma-associated antigen, Kahn, 1991) epithelial tumor cell mucins, leukemia associated mucins, carcinoembryonic antigen, or any other mucin associated with abnormal cells according to known characteristics of cancer associated mucins or abnormal mucins, such as abberant glycosylation (Hakomori, 1989, and Singhal, 1990).

MUC1 Epitopes

The human MUC1 gene product has been referred to by various names, including MAM6, milk mucin; human milk fat globule antigen (HMFG); human mammary epithelial antigen, CA 15-3, CA 27.29; episialin; and polymorphic epithelial mucin (PEM) (reviewed in Taylor-Papadimitriou et al, 1988) (for complete cites to the incompletely cited references in this section, see Longenecker, et al., Ser. No. 08/229,606). This mucin is strongly expressed on human breast (Gendler et al, 1988), pancreatic (Lan et al, 1990) and certain ovarian cancer cells (Layton et al, 1990). Although the MUC1 encoded mucins expressed on various cancers contain the same tandem repeat core peptide sequence, glycosylation differences do exist (Gendler et al, 1988; Lan et al, 1990). Because of underglycosylation in cancer cells, MUC-1 molecules on cancer cells express cryptic epitopes which are not expressed (i.e, are cryptic) on normal epithelial cells.

MUC1 is the first cancer-associated mucin gene to be cloned and mapped (Gendler et al, 1990), and has recently been transfected into a murine mammary cell line, 410.4 (Lalani et al, 1991). MUC1 transfected 410.4 cells express the MUC1 gene product on the cell surface.

The pattern of glycosylation is similar to, but different from, malignant cell derived mucins expressing the same cryptic peptide epitopes as expressed by human cancer associated MUC1 (Taylor-Papadimitriou et al, 1988). Lalani and co-workers (1991) have examined the immunogenicity of the 410.4 transfectants in mice. These workers demonstrated that mice which rejected a low dose of transfected 410.4 cells did not develop tumors after a subsequent transplant of a high dose of transfected 410.4 cells although no effect on tumor development of untransfected wild type 410.4 cells was seen (Taylor-Papadimitriou et al, 1988). (For complete cites, see Longenecker5-USA, and see also refs 4-11 thereof).

It has been shown that cancer vaccines composed of synthetic peptide antigens which mimic cryptic MUC-1 peptide sequences on cancer cells are able to induce effective anti-cancer immunotherapy against MUC-1 expressing tumor cells in a murine model Finn and co-workers have shown that cancer patients are able to produce specific non-MHC restricted cytotoxic T-lymphocytes (CTL) which recognize peptide epitopes expressed on MUC-1 molecules on cancer cells. (See refs. 12 and 53-55 of U.S. Ser. No. 08/229,606, docket Longenecker5-USA, hereby incorporated by reference.) Indeed the MUC1 sequence SAPDTRP (AAs 11-17 of SEQ ID NO: 2) has been shown to be both a T-and a B-cell epitope. It has been demonstrated that the immunization of chimpanzees with synthetic MUC-1 antigens induces the development of specific antibodies and CMI against MUC-1.

The human epithelial mucin MUC1 is over-expressed in more than 90% of carcinomas of the breast, ovary and pancreas, and in those tumors it is aberrantly glycosylated. The SM3 antibody binds the core protein of MUC1; it also binds the tumor glycoproteins, presumably because the SM3 epitope is exposed as a result of the aforementioned aberrant glycosylation.

The amino acid sequence of Human MUC1 is available in the SWISS-PROT database as P15941. The number of repeats is highly polymorphic. It varies from 21 to 125 in the northern european population. The most frequent alleles contains 41 and 85 repeats. The tandemly repeated icosapeptide underlies polymorphism at three positions, as shown by brackets: PAPGSTAP[P/A/Q/T]AHGVTSAP[D/E][T/S]R (SEQ ID NO: 5). The common polymorphisms are the coordinated double mutation DT—>ES and the single replacements P—>A, P—>Q and P—>T. The most frequent replacement DT>ES occurs in up to 50% of the repeats. For Mouse MUC1, see SWISS-PROT Q02496.

Moller, et al., Eur. J. Biochem. 269:1444-55 (March 2002) has used NMR spectroscopy to study the binding of the SM3 antibody to the pentapeptide MUC1 epitope PDTRP and to the related glycopentapeptide in which the threonine is O-lined to alpha-d-GalNAc. Moller found that the PDT interacted with the SM3 antibody more strongly than did the RP, suggesting that the RP would be more tolerant of mutation. In contrast, the glycopeptide interacted with SM3 using all of its amino acids, although the strongest effect was with the Prol. Docking studies were conducted; these could be performed with mutant peptides for which 3D structures are deducible or determined.

Hiltbold, et al., Cancer Res., 58:5066-70 (1998) showed that CD4+ T-cells primed in vitro with a synthetic MUC1 peptide of 100 amino acids, representing five unglycosylated tandem repeats, and presented by dendritic cells, produced IFN-gamma and had moderate cytolytic activity. They also identified a core peptide sequence, PGSTAPPAHGVT (SEQ ID NO: 6), which elicits this response when it is presented by HLA-DR3.

Heukamp, et al., Int. J. Cancer, 91:385-92 (2001) elicited peptide-specific CTL immunity in A2/K(b) transgenic mice with three MUC1-derived peptides that map outside the variable number tandem repeat region. These peptides were MUC(79-87)(TLAPATEPA)(SEQ ID NO: 7), MUC(167-175)(ALGSTAPPV)(SEQ ID NO: 8) and MUC(264-72)(FLSFHISNL)(SEQ ID NO: 9). All comply with the peptide binding motif for HLA-A*0201.

Engelmann, et al., J. Biol. Chem. 276:27764-9 (July 2001) report that there are three sequence variants in the tandem repeat region of MUC1. Variant 1 replaced DT with ES.

Soares et al., J. Immunol. 166: 6555-63 (June 2001) used a seven tandem repeat MUC1 peptide to elicit an immune response. If the peptide was delivered on dendritic cells, it only elicited T cell immunity. If injected together with soluble peptide, Ab production was also triggered.

Von Mensdorff-Pouilly et al., J. Clin. Oncol. 18:574-83 (February 2000) used a MUC1 triple tandem repeat peptide conjugated to BSA in an immunoassay of anti-MUC1 antibody levels in breast cancer patients.

Denton, et al., Pept. Res. 7:258-64 (September/October 1994), colinearly liked a MUC1 mucin B cell peptide epitope to a known murine T cell epiope in both T-B and B-T orientations.

Brossart et al., Blood, 93:4309-17 (June 1999) analyzed the MUC1 amino acid sequence and identified two novel peptides with a high binding probability to the HLA-A2 molecule. One was from the variable tandem repeat region, and the other from outside it.

Carmon, et al., Int. J. Cancer, 85:391-7 (February 2000) evaluated the anti-tumor potential of HLA-A2.1 motif-selected peptides from non-tandem repeat regions of the molecule. See also Pietersz et al., Vaccine, 18:2059-71 (April 2000).

Keil, et al. Angew. Chem. Int. Ed. Engl. 40:366-9(January 2001) conjugated a MUC1 epitope to a tetanus toxin epitope.

Schreiber, et al., Anticancer Res. 20:3093-8(September/October 2000) showed that the peptide with five MUC1 tandem repeats had three times the binding affinity for the bacterial heat shock protein DnaK (mammalian heat shock proteins are involved in antigen processing) as did the peptide with just one repeat.

Von Mensdorff-Pouilly et al., Int. J. Cancer, 86:702-12 (June 2000) reported that the most frequent minimal epitopic sequences of natural MUC1 IgG and IgM antibodies were RPAPGS (AAs 9-14 of SEQ ID NO: 10), PPAHGVT (SEQ ID NO: 11; equivalent to AAs 17-20 followed by AAS 1-3 of SEQ ID NO:10) and PDTRP (AAs 6-10 of SEQ ID NO: 10). MUC1 peptide vaccination induced high titers of IgM and IgG antibodies predominantly directed, respectively, to the PDTRPAP (AAs6-12 of SEQ ID NO: 10) and the STAPPAHGV (AAs 1-9 of SEQ ID NO:2) sequences of the tandem repeat. Natural MUC abs from breast cancer patients reacted more strongly with GalNac-glycosylated peptides than with unglycosylated peptides.

See also EP Appl 1,182,210; Sandrin, U.S. Pat. No. 6,344,203; Finn, U.S. Pat. No. 5,744,144.

See also, Petrakou, et al., “Epitope Mapping of Anti-MUC1 Mucin protein Core Monoclonal Antibodies” (21-29); Imai, et al., “Epitope Characterization of MUC1 Antibodies” (30-34), Schol, et al., “Epitope Fingerprinting Using Overlapping 20-mer peptides of the MUC1 Tandem repeat sequence” (35-45), and Blockzjil, “Epitope characterization of MUC1 Antibodies” (46-56), all in ISOBM TD-4 International Workshop on Monoclonal Antibodies against MUC1 November 1996), reprinted in Tumor Biology, 19 Suppl. 1: 1-152 (1998).

See also Von Mensdorff-Pouilly, et al., “Human MUC1 mucin: a multifacted glycoprotein,” Int J. Biol. Markers, 15:343-56 (2000)

The present invention therefore contemplates immunogens which comprise at least one native B and/or T cell epitope of MUC1, or at least one mutant epitope substantially identical to such a native epitope. It may further comprise additional MUC1 sequence which is not part of an epitope.

Preferably, theimmunogen comprises both a B cell epitope and a T cell epitope of MUC1 (which, in each case, may be a natural epitope or an allowed mutant thereof), and these epitopes may be identical, overlapping, or distinct.

T and B cell epitopes of an antigen may overlap. For example, in the case of MUC-1, SAPDTRP (AAs 4-10 of SEQ ID NO:10) is a T-cell epitope, while PDTRP (AAs 6-10 of SEQ ID NO:10) is merely a B-cell epitope.

It may further comprise additional B cell epitopes, and/or additional T cell epitopes. The B cell epitopes may be the same or different, and likewise the T cell epitopes may be the same or different.

If the immunogen of the present invention comprises a MUC1-related sequence at least substantially identical to a MUC1 sequence of at least five amino acids, the MUC1-related sequence may comprise one or more glycosylation sites found in the corresponding MUC1 sequence. It may differ from the corresponding MUC1 sequence in the number of potential glycosylation sites, as a result of mutation, or it may have the same number of potential glycosylation sites.

The potential glycosylation sites may be (1) sites actually glycosylated in the MUC1-derived tumor glycoprotein, (2) sites potentially glycosylatable but not actually glycosylated in that tumor glycoprotein, and/or (3) sites foreign to said glycoprotein. Likewise, the actual glycosylation sites may be (1) sites actually glycosylated in the MUC1-derived tumor glycoprotein, (2) sites potentially glycosylatable but not actually glycosylated in that tumor glycoprotein, and/or (3) sites foreign to said glycoprotein. None, one, some or all of the glycosylation sites normally glycosylated in the MUC1-derived tumor glycoprotein may be glycosylated in the immunogen of the present invention.

MUC1 is a polymorphic antigen characterized by a variable number (typically 21-125, especially 41 or 85) of perfect and imperfect repeats of the following sequence:

| | |
|---|---|
| GVTSAPDTRPAPGSTAPPAH | (SEQ ID NO: 10) |

Since there are multiple repeats of this sequence, the starting point shown is arbitrary, and an epitope may bridge two repeats.
Consequently, the immunogens of the present invention may comprise the aforementioned complete repeat sequence or a cyclic permutation thereof. Moreover, they may comprise two or more copies of the aforementioned repeat or a cyclic permutation thereeof. Thus, in compounds 1a and 1b, there are two copies of a cyclic permutation (starting at TSA . . . and ending with HGV) of the above sequence, followed by the unrelated SSL sequence.

Each MUC1 epitope in question may correspond to an epitope of the variable tandem repeat region, or to an epitope outside that region. The former include RPAPGS (AAs 9-14 of SEQ ID NO:10), PPAHGVT (SEQ ID NO:11) and PDTRP (AAs 6-10 of SEQ ID NO:10). The sequence PDTRPAPGS (AAs 6-14 of SEQ ID NO:10) is of particular interest, as it includes two overlapping epitopes. The PDTRP sequence forms the tip of a protruding knob exposed to solvents and forming a stable type II beta-turn.

The non-VNTR region epitopes include MUC(79-87)(TLAPATEPA)(SEQ ID NO:7), MUC(167-175)(ALGSTAPPV)(SEQ ID NO:8) and MUC(264-72)(FLSFHISNL)(SEQ ID NO:9).

Preferably, the immunogen comprises the polymorphic epitope P[D/E][T/S]RP or a substantially identical mutant thereof. More preferably it comprises PDTRP (AAs 6-10 of SEQ ID NO:10) or a substantially identical mutant thereof.

In some embodiments, the immunogen comprises at least one 20 amino acid sequence (an effective tandem repeat) which differs solely by one or more conservative substitutions and/or a single nonconservative substitution from a tandem repeat of MUC1, and comprises an epitope of the variable tandem repeat region of MUC1 (either identically, or an allowed mutant). Preferably, it differs solely, if at all, by conservative substitutions, more preferably, by no more than a single conservative substitutions, and most preferably, is identical to such a tandem repeat. It should be noted that the tandem repeats of MUC1 are imperfect and hence the sequence could be identical to one repeat but not to another. Also, there are allelic variations in these repeats, and so the sequence could be identical to the sequence for one allele and not for another.

In a subset of these embodiments, the immunogen comprises a plurality of nonoverlapping effective tandem repeats, such as two (for a total of 40 amino acids), three (for a total of 60 amino acids), four, five, six, seven or eight. These effective tandem repeats may, but need not be, identical to each other. (In contrast, note that in the natural human MUC1 mucin, the number of repeats is typically 21-125.)

Besides one or more effective tandem repeats, the peptide portion of the immunogen may comprise additional amino acid subsequences. If so, these subsequences may comprise additional epitopes, which may be MUC1 variable tandem repeat region epitopes (falling short of a effective tandem repeat), MUC1 epitopes from outside that region, or epitopes of other cancer antigens. It may also include an immunomodulatory element, see Longenecker, et al., Ser. No. 08/229,606.

Preferably, one or more of the serines and/or threonines of the MUC1 tandem repeat are glycosylated, preferably with Tn or sialyl. Tn. In the natural human MUC1 mucin, there are five normal glycosylation sites per repeat. In normal MUC1, an average of 2.6 of these five sites is in fact occupied. The average number of glycosylated amino acids per repeat may be less than, the same as, or greater than the “natural” value.

Preferably, the immunogen is lipidated, as disclosed in Koganty, et al., U.S. Provisional Appl. 60/377,595, filed May 6, 2002 (docket: Koganty4.1-USA), incorporated by reference. More preferably, it comprises, in its C-terminal region, the sequence SSL, where both serines are lipidated.

Identification of Naturally Occurring Epitopes

Other naturally occurring epitopes may be identified by the methods set forth in Koganty et al., U.S. Prov. 60/377,595 (Koganty4.1-USA).

Mutant Epitopes

Generally speaking, in addition to epitopes which are identical to the naturally occurring disease- or tumor-specific epitopes, the present invention embraces epitopes which are different from but substantially identical with such epitopes, and therefore disease- or tumor-specific in their own right. It also includes epitopes which are not substantially identical to a naturally occurring epitope, but which are nonetheless cross-reactive with the latter as a result of a similarity in 3D conformation.

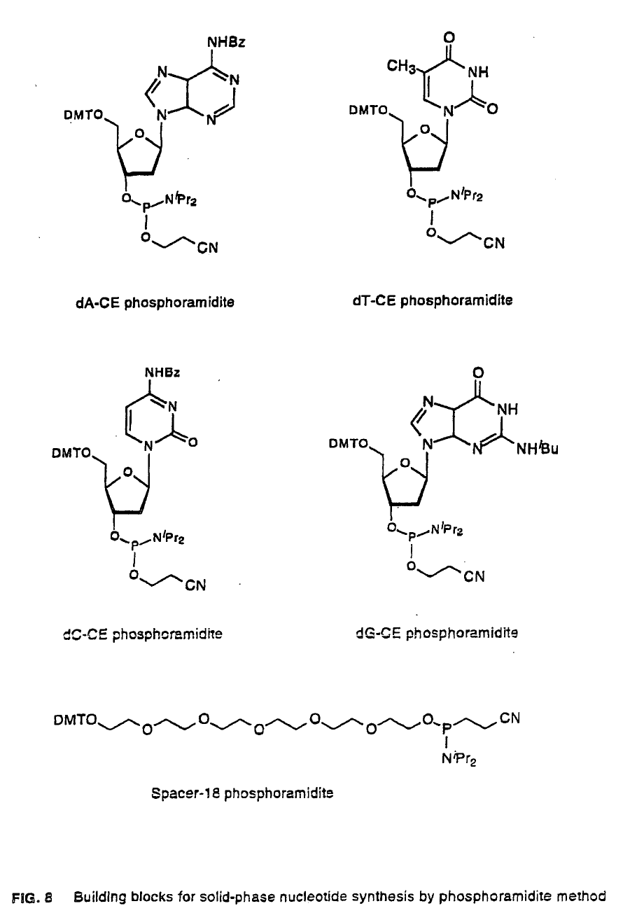

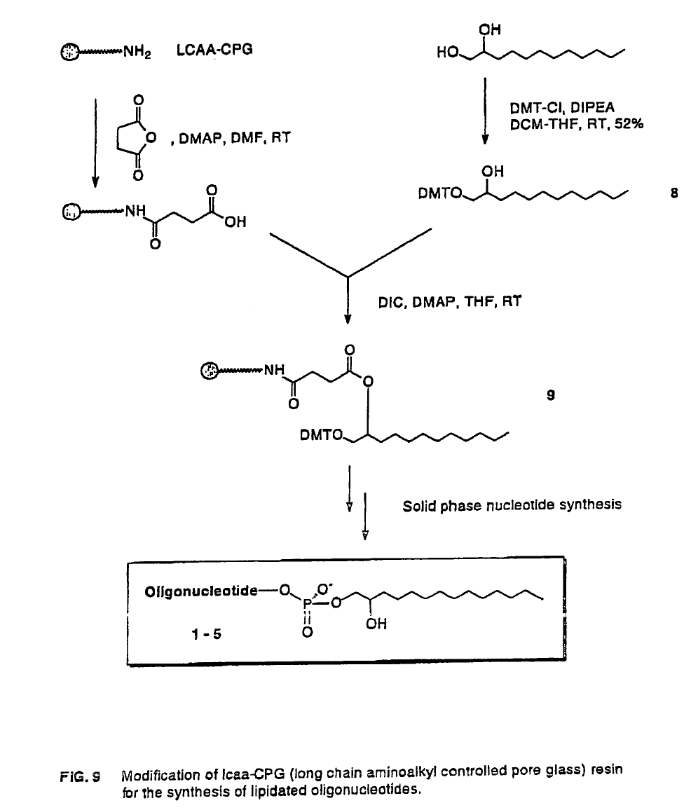

One class of allowable modifications of the amino acid sequence of a peptide moiety are amino acid substitutions. Conservative substitutions replace an amino acid with another of like size, charge and polarity; these are less likely to substantially alter the conformation of the peptide. The types of substitutions which may be made in the protein or peptide molecule of the present invention may be based on analysis of the frequencies of amino acid changes between a homologous protein of different species, such as those presented in Table 1-2 of Schulz et al., supra and FIGS. 3-9 of Creighton, supra. Based on such an analysis, conservative substitutions are defined herein as exchanges within one of the following five groups:

TABLE V

| | |
|---|---|
| 1. | Small aliphatic, nonpolar or slightly polar |
| | residues: Ala, Ser, Thr (Pro, Gly); |
| 2. | Polar, negatively charged residues and their |
| | amides: Asp, Asn, Glu, Gln; |
| 3. | Polar, positively charged residues: |
| | His, Arg, Lys; |
| 4. | Large aliphatic, nonpolar or slightly polar |
| | residues: |
| | Met, Leu, Ile, Val (Cys); and |
| 5. | Large aromatic residues: Phe, Tyr, Trp. |
| | Groups 1-3 are somewhat related and mutations within that |
| | set may be considered semi-conservative. Similarly, |
| | mutations within groups 4-5 may be considered semi- |
| | conservative. |

Residues Pro, Gly and Cys are parenthesized because of their special role in protein architecture. Pro imparts rigidity to the peptide chain, and has a tendency to interfere with alpha helix formation. Gly imparts flexibility to the peptide chain, and is often found in “loops” between alpha helices or beta strands. The thiol groups of cysteine residues can be oxidized to form disulfide bonds between nonadjacent cysteinyl residues.

Within the foregoing groups, the following substitutions are considered “highly conservative”:

Asp/Glu

His/Arg/Lys

Phe/Tyr/Trp

Met/Leu/Ile/Val

Semi-conservative substitutions are defined to be exchanges between two of groups (I)-(V) above which are limited to supergroup (A), comprising (I), (II) and (III) above, or to supergroup (B), comprising (IV) and (V) above. Also, Ala is considered a semi-conservative substitution for all non group I amino acids.

An epitope is considered substantially identical to a reference epitope (e.g., a naturally occurring epitope) if it has at least 10% of an immunological activity of the reference epitope and differs from the reference epitope by no more than one non-conservative substitution (except as provided below). Preferably, any non-conservative substitution is a semi-conservative substitution. Preferably, there are no non-conservative substitutions.

There may be any number of conservative substitutions. Preferably, there are no more than three such substitutions, more preferably, not more than two, and still more preferably, not more than one.

It will be appreciated that highly conservative substitutions are less likely to affect activity than other conservative substitutions, conservative substitutions are less likely to affect activity than merely semi-conservative substitutions, and semi-conservative substitutions less so than other non-conservative substitutions. In addition, single substitutions are less likely to affect activity than are multiple mutations.

Although a substitution mutant, either single or multiple, of the peptides of interest may not have quite the potency of the original peptide, such a mutant may well be useful.

Substitutions are not limited to the genetically encoded, or even the naturally occurring amino acids. When the epitope is prepared by peptide synthesis, the desired amino acid may be used directly. Alternatively, a genetically encoded amino acid may be modified by reacting it with an organic derivatizing agent that is capable of reacting with selected side chains or terminal residues.

A non-genetically encoded amino acid is considered a conservative substitution for a genetically encoded amino acid if it is more similar in size (volume) and hydrophobicity (lipophilicity) to the original amino acid, and to other amino acids in the same exchange group, than it is to genetically encoded amino acids belonging to other exchange groups.

Methods of identifying mutant epitopes are further described in Koganty et al., U.S. Prov. 60/377,595 (Koganty4.1-USA).

Analogues

Also of interest are analogues of the disclosed compounds.

Analogues may be identified by assigning a hashed bitmap structural fingerprint to the compound, based on its chemical structure, and determining the similarity of that fingerprint to that of each compound in a broad chemical database. The fingerprints are determined by the fingerprinting software commercially distributed for that purpose by Daylight Chemical Information Systems, Inc., according to the software release current as of Jan. 8, 1999. In essence, this algorithm generates a bit pattern for each atom, and for its nearest neighbors, with paths up to 7 bonds long. Each pattern serves as a seed to a pseudorandom number generator, the output of which is a set of bits which is logically ored to the developing fingerprint. The fingerprint may be fixed or variable size.

The database may be SPRESI'95 (InfoChem GmbH), Index Chemicus (ISI), MedChem (Pomona/Biobyte), World Drug Index (Derwent), TSCA93(EPA) May bridge organic chemical catalog (Maybridge), Available Chemicals Directory (MDLIS Inc.), NCI96 (NCI), Asinex catalog of organic compounds (Asinex Ltd.), or IBIOScreen SC and NP (Inter BioScreen Ltd.), or an inhouse database.

A compound is an analogue of a reference compound if it has a daylight fingerprint with a similarity (Tanamoto coefficient) of at least 0.85 to the Daylight fingerprint of the reference compound.

A compound is also an analogue of a reference compound id it may be conceptually derived from the reference compound by isosteric replacements.

Homologues are compounds which differ by an increase or decrease in the number of methylene groups in an alkyl moiety.

Classical isosteres are those which meet Erlenmeyer's definition: “atoms, ions or molecules in which the peripheral layers of electrons can be considered to be identical”. Classical isosteres include

| Monovalents | Bivalents | Trivalents | Tetra | Annular |
|---|---|---|---|---|
| F, OH, NH₂, CH₃ | —O— | —N═ | ═C═ | —CH═CH— |
| | | | ═Si═ | |
| Cl, SH, PH₂ | —S— | —P═ | —N+═ | —S— |
| Br | —Se— | —As— | ═P+═ | —O— |
| I | —Te— | —Sb— | ═As+═ | —NH— |
| | | —CH═ | ═Sb+═ | |

Nonclassical isosteric pairs include —CO— and —SO₂—, —COOH and —SO₃H, —SO₂NH₂ and —PO(OH)NH₂, and —H and —F, —OC(═O)— and C(═O)O—, —OH and —NH₂.

Characterizing the Immune Response

A cell-mediated immune response may be assayed in vitro or in vivo. The conventional in vitro assay is a T cell proliferation assay. A blood sample is taken from an individual who suffers from the disease of interest, associated with that disease, or from a vaccinated individual. The T cells of this individual should therefore be primed to respond to a new exposure to that antigen by proliferating. Proliferation requires thymidine because of its role in DNA replication.

Generally speaking, T cell proliferation is much more extensive than B cell proliferation, and it may be possible to detect a strong T cell response in even an unseparated cell population. However, purification of T cells is desirable to make it easier to detect a T cell response. Any method of purifying T cells which does not substantially adversely affect their antigen-specific proliferation may be employed. In our preferred procedure, whole lymphocyte populations would be first obtained via collection (from blood, the spleen, or lymph nodes) on isopycnic gradients at a specific density of 10.7, ie Ficoll-Hypague or Percoll gradient separations. This mixed population of cells could then be further purified to a T cell population through a number of means. The simplest separation is based on the binding of B cell and monocyte/macrophage populations to a nylon wool column. The T cell population passes through the nylon wool and a >90% pure T population can be obtained in a single passage. Other methods involve the use of specific antibodies to B cell and or monocyte antigens in the presence of complement proteins to lyse the non-T cell populations (negative selection). Still another method is a positive selection technique in which an anti-T cell antibody (CD3) is bound to a solid phase matrix (such as magnetic beads) thereby attaching the T cells and allowing them to be separated (e.g., magnetically) from the non-T cell population. These may be recovered from the matrix by mechanical or chemical disruption.

Once a purified T cell population is obtained it is cultured in the presence of irradiated antigen presenting cells (splenic macrophages, B cells, dendritic cells all present). (These cells are irradiated to prevent them from responding and incorporating tritiated thymidine). The viable T cells (100,000-400,000 per well in 100 μl media supplemented with IL2 at 20 units) are then incubated with test peptides or other antigens for a period of 3 to 7 days with test antigens at concentrations from 1 to 100 μg/mL.

At the end of the antigen stimulation period a response may be measured in several ways. First the cell free supernatants may be harvested and tested for the presence of specific cytokines. The presence of α-interferon, IL2 or IL12 are indicative of a Th helper type 1 population response. The presence of IL4, IL6 and IL10 are together indicative of a T helper type 2 immune response. Thus this method allows for the identification of the helper T cell subset.

A second method termed blastogenesis involves the adding tritiated thymidine to the culture (e.g., 1 μcurie per well) at the end of the antigen stimulation period, and allowing the cells to incorporate the radiolabelled metabolite for 4-16 hours prior to harvesting on a filter for scintillation counting. The level of radioactive thymidine incorporated is a measure of the T cell replication activities. Negative antigens or no antigen control wells are used to calculated the blastogenic response in terms of a stimulation index. This is CPM test/CPM control. Preferably the stimulation index achieved is at least 2, more preferably at least 3, still more preferably 5, most preferably at least 10. CMI may also be assayed in vivo in a standard experimental animal, e.g., a mouse. The mouse is immunized with a priming antigen. After waiting for the T cells to respond, the mice are challenged by footpad injection of the test antigen. The DTH response (swelling of the test mice is compared with that of control mice injected with, e.g., saline solution.

Preferably, the response is at least 0.10 mm, more preferably at least 0.15 mm, still more preferably at least 0.20 mm, most preferably at least 0.30 mm.

The humoral immune response, in vivo, is measured by withdrawing blood from immunized mice and assaying the blood for the presence of antibodies which bind an antigen of interest. For example, test antigens may be immobilized and incubated with the samples, thereby capturing the cognate antibodies, and the captured antibodies then measured by incubating the solid phase with labeled anti-isotypic antibodies.

Preferably, the humoral immune response, if desired, is at least as strong as that represented by an antibody titer of at least 1/100, more preferably at least 1/1000, still more preferably at least 1/10,000.

Pharmaceutical Compositions and Methods

Subjects

The recipients of the vaccines of the present invention may be any vertebrate animal which can acquire specific immunity via a humoral or cellular immune response.

Among mammals, the preferred recipients are mammals of the Orders Primata (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

The preferred animal subject of the present invention is a primate mammal. By the term “mammal” is meant an individual belonging to the class Mammalia, which, of course, includes humans. The invention is particularly useful in the treatment of human subjects, although it is intended for veterinary uses as well. By the term “non-human primate” is intended any member of the suborder Anthropoidea except for the family Hominidae. Such non-human primates include the superfamily Ceboidea, family Cebidae (the New World monkeys including the capuchins, howlers, spider monkeys and squirrel monkeys) and family Callithricidae (including the marmosets); the superfamily Cercopithecoidea, family Cercopithecidae (including the macaques, mandrills, baboons, proboscis monkeys, mona monkeys, and the sacred hunaman monkeys of India); and superfamily Hominoidae, family Pongidae (including gibbons, orangutans, gorillas, and chimpanzees). The rhesus monkey is one member of the macaques.

Immunostimulatory Compositions

The present invention contemplates use of at least one pharmaceutical composition comprising at least one immunostimulatory oligonucleotide molecule as disclosed above.

This immunostimulatory composition may be used by itself to nonspecifically increase or otherwise alter the immunological state of readiness of a subject.

Thus, it may be administered, other than in conjunction with a pharamaceutically administered immunogen, to enhance the innate immune response to an immunogen presented by a microbial or non-microbial pathogen, or by a cancer. It may thereby be used to increase a subject's resistance to contracting a disease, or to treat an existing disease. In these uses, it optionally may be used in conjunction with a non-immunological agent directed against said disease, e.g., an antibiotic for treating a bacterial infection.

Or it may be used as part of or otherwise in conjunction with a pharmaceutical composition comprising an immunogen, which elicits a specific immune response protective against a disease, in which case it will have the effect of potentiating that disease-specific response. Again, these immunological agents may be used in conjunction with non-immunological agents directed against said disease.

Some of the pharmaceutical compositions of the present invention comprise at least one immunogen in an amount effective to elicit a protective immune response. The response may be humoral, cellular, or a combination thereof. The composition may comprise a plurality of immunogens.

At least one immunogen is preferably either a glycolipopeptide which is immunogenic per se, or a glycolipopeptide which is immunogenic as a result of its incorporation into a liposome. Glycolipopeptides are described in U.S. Provisional Appl. 60/377,595, filed May 6, 2002 (docket Koganty4.1-USA), incorporated by reference in its entirety.

The immunogen may be administered before, after, or at the same time as the immunostimulatory oligonucleotide is administered. If administered simultaneously, they may be presented in a single composition, or in different compositions; different compositions can be administered by different routes. The oligonucleotide molecule then is considered to be an adjuvant, although other adjuvants may also be used, and although it may have useful activities other than that of nonspecific immunostimulation.

It is furthermore possible to combine the functions of the immunogen and the immunostimulatory oligonucleotide into a single molecule comprising at least one CxG dinucleotide unit, at least one lipophilic group, and at least one epitope.

The pharmaceutical composition preferably further comprises a liposome. Preferred liposomes include those identified in Jiang, et al., PCT/US00/31281, filed Nov. 15, 2000 (our docket JIANG3A-PCT), and Longenecker, et al., Ser. No. 08/229,606, filed Apr. 12, 1994 (our docket LONGENECKER5-USA, and PCT/US95/04540, filed Apr. 12, 1995 (our docket LONGENECKER5-PCT).

The composition may comprise antigen-presenting cells, and in this case the immunogen may be pulsed onto the cells, prior to administration, for more effective presentation.

The composition may contain auxiliary agents or excipients which are known in the art. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Katzung, ed. Basic and Clinical Pharmacology, Fifth Edition, Appleton and Lange, Norwalk, Conn. (1992), which references and references cited therein, are entirely incorporated herein by reference.

A pharmaceutical composition may further comprise an adjuvant, other than the instant immunostimulatory lipidated oligonucleotides, to nonspecifically enhance the immune response. Some adjuvants potentiate both humoral and cellular immune response, and other s are specific to one or the other. Some will potentiate one and inhibit the other. The choice of adjuvant is therefore dependent on the immune response desired.

A pharamceutical composition may include other immunomodulators, such as cytokines which favor or inhibit either a cellular or a humoral immune response, or inhibitory antibodies against such cytokines.

A pharmaceutical composition according to the present invention may comprise at least one cancer chemotherapeutic compound, such as one selected from the group consisting of an anti-metabolite, a bleomycin peptide antibiotic, a podophyllin alkaloid, a Vinca alkaloid, an alkylating agent, an antibiotic, cisplatin, or a nitrosourea.

A pharmaceutical composition may comprise at least one viral chemotherapeutic compound selected from gamma globulin, amantadine, guanidine, hydroxybenzimidazole, interferon-α, interferon-β, interferon-γ, thiosemicarbarzones, methisazone, rifampin, ribvirin, a pyrimidine analog, a purine analog, foscarnet, phosphonoacetic acid, acyclovir, dideoxynucleosides, or ganciclovir. See, e.g., Katzung, supra, and the references cited therein on pages 798-800 and 680-681, respectively, which references are herein entirely incorporated by reference.

A pharmaceutical composition may comprise at least one antibacterial agent. Such agents include penicillins, cephalosporins, chloramphenicol, tetracyclines, aminoglycosides, polymyxins, sulfonamides, and trimethoprim. Some specific agents of particular interest are amoxicillin; ampicillin; benzylpenicillin; chloramphenicol; clindamycin; enrofloxacin; erythromycin; lincomycin; and rifampicin.

A pharmaceutical composition may comprise at least one anti-parasitic agent. Anti-parasitic agents include agents suitable for use against arthropods, helminths (including roundworns, pinworms, threadworms, hookworms, tapeworms, whipworms, and Schistosomes), and protozoa (including amebae, and malarial, toxoplasmoid, and trichomonad organisms). Examples include thiabenazole, various pyrethrins, praziquantel, niclosamide, mebendazole, chloroquine HCl, metronidazole, iodoquinol, pyrimethamine, mefloquine HCl, and hydroxychloroquine Hcl.

A pharmaceutical composition may comprise at least one agent which ameliorates a symptom of the disease. Symptoms include, e.g., pain and fever.

These various active agents can be administered as part of the same composition as said immunogen or said immunostimulatory lipidated oligonucleotide molecule, as part of a separate composition administered simultaneously, or as part of a separate composition administered at a different time to the subject.

Pharmaceutical Purposes

A purpose of the invention is to protect subjects against a disease. The term “protection”, as in “protection from infection or disease”, as used herein, encompasses “prevention,” “suppression” or “treatment.” “Prevention” involves administration of a Pharmaceutical composition prior to the induction of the disease. “Suppression” involves administration of the composition prior to the clinical appearance of the disease. “Treatment” involves administration of the protective composition after the appearance of the disease. Treatment may be ameliorative or curative.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.” See, e.g., Berker, supra, Goodman, supra, Avery, supra and Katzung, supra, which are entirely incorporated herein by reference, including all references cited therein.

The “protection” provided need not be absolute, i.e., the disease need not be totally prevented or eradicated, provided that there is a statistically significant improvement (p=0.05) relative to a control population. Protection may be limited to mitigating the severity or rapidity of onset of symptoms of the disease. An agent which provides protection to a lesser degree than do competitive agents may still be of value if the other agents are ineffective for a particular individual, if it can be used in combination with other agents to enhance the level of protection, or if it is safer than competitive agents.

The effectiveness of a treatment can be determined by comparing the duration, severity, etc. of the disease post-treatment with that in an untreated control group, preferably matched in terms of the disease stage.

The effectiveness of a prophylaxis will normally be ascertained by comparing the incidence of the disease in the treatment group with the incidence of the disease in a control group, where the treatment and control groups were considered to be of equal risk, or where a correction has been made for expected differences in risk.

In general, prophylaxis will be rendered to those considered to be at higher risk for the disease by virtue of family history, prior personal medical history, or elevated exposure to the causative agent.

Pharmaceutical Administration

At least one protective agent of the present invention may be administered by any means that achieve the intended purpose, using a pharmaceutical composition as previously described.

Administration may be oral or parenteral, and, if parenteral, either locally or systemically. For example, administration of such a composition may be by various parenteral routes such as subcutaneous, intravenous, intradermal, intramuscular, intraperitoneal, intranasal, transdermal, or buccal routes. Parenteral administration can be by bolus injection or by gradual perfusion over time. A preferred mode of using a pharmaceutical composition of the present invention is by subcutaneous, intramuscular or intravenous application. See, e.g., Berker, supra, Goodman, supra, Avery, supra and Katzung, supra, which are entirely incorporated herein by reference, including all references cited therein.

A typical regimen for preventing, suppressing, or treating a disease or condition which can be alleviated by an immune response by active specific immunotherapy, comprises administration of an effective amount of a pharmaceutical composition as described above, administered as a single treatment, or repeated as enhancing or booster dosages, over a period up to and including between one week and about 24 months.

It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. This will typically involve adjustment of a standard dose, e.g., reduction of the dose if the patient has a low body weight. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Chabner et al., supra; De Vita et al., supra; Salmon, supra; Schroeder et al., supra; Sartorelli et al., supra; and Katsung, supra, which references and references cited therein, are entirely incorporated herein by reference.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow, et al., eds., The Merck Manual, 15th edition, Merck and Co., Rahway, N.J., 1987; Goodman, et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered in multiple doses (which may be the same or different) or in a single dose, according to an immunization schedule, which may be predetermined or ad hoc. The schedule is selected so as to be immunologically effective, i.e., so as to be sufficient to elicit an effective immune response to the antigen and thereby, possibly in conjunction with other agents, to provide protection. The doses adequate to accomplish this are defined as “therapeutically effective doses.” (Note that a schedule may be immunologically effective even though an individual dose, if administered by itself, would not be effective, and the meaning of “therapeutically effective dose” is best interpreted in the context of the immunization schedule.) Amounts effective for this use will depend on, e.g., the peptide composition, the manner of administration, the stage and severity of the disease being treated, the weight and general state of health of the patient, and the judgment of the prescribing physician.

Typically, the daily dose of an active ingredient of a pharmaceutical, for a 70 kg adult human, is in the range of 10 nanograms to 10 grams. For immunogens, a more typical daily dose for such a patient is in the range of 10 nanograms to 10 milligrams, more likely 1 microgram to 10 milligrams. However, the invention is not limited to these dosage ranges.

It must be kept in mind that the compositions of the present invention may generally be employed in serious disease states, that is, life-threatening or potentially life threatening situations. In such cases, in view of the minimization of extraneous substances and the relative nontoxic nature of the peptides, it is possible and may be felt desirable by the treating physician to administer substantial excesses of these peptide compositions.

The doses may be given at any intervals which are effective. If the interval is too short, immunoparalysis or other adverse effects can occur. If the interval is too long, immunity may suffer. The optimum interval may be longer if the individual doses are larger. Typical intervals are 1 week, 2 weeks, 4 weeks (or one month), 6 weeks, 8 weeks (or two months) and one year. The appropriateness of administering additional doses, and of increasing or decreasing the interval, may be reevaluated on a continuing basis, in view of the patient's immunocompetence (e.g., the level of antibodies to relevant antigens).

The appropriate dosage form will depend on the disease, the immunogen, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

The immunogen may be delivered in a manner which enhances immunogenicity, e.g., delivering the antigenic material into the intracellular compartment such that the “endogenous pathway” of antigen presentation occurs. For example, the immunogen may be entrapped by a liposome (which fuses with the cell), or incorporated into the coat protein of a viral vector (which infects the cell).

Another approach, applicable when the immunogen is a peptide, is to inject naked DNA encoding the immunogen into the host, intramuscularly. The DNA is internalized and expressed.

It is also possible to prime autologous PBLs with the compositions of the present invention, confirm that the PBLs have manifested the desired response, and then administer the PBLs, or a subset thereof, to the subject.

Liposome Formulations

Liposomes are microscopic vesicles that consist of one or more lipid bilayers surrounding aqueous compartments. See e.g., Bakker-Woudenberg et al., Eur. J. Clin. Microbiol. Infect. Dis. 12 (Suppl. 1): S61 (1993) and Kim, Drugs, 46: 618 (1993). Because liposomes can be formulated with bulk lipid molecules that are also found in natural cellular membranes, liposomes generally can be administered safely and are biodegradable.

Liposomes are globular particles formed by the physical self-assembly of polar lipids, which define the membrane organization in liposomes. Liposomes may be formed as uni-lamellar or multi-lamellar vesicles of various sizes. Such liposomes, though constituted of small molecules having no immunogenic properties of their own, behave like macromolecular particles and display strong immunogenic characteristics.

A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019369, incorporated herein by reference.

Depending on the method of preparation, liposomes may be unilamellar or multilamellar, and can vary in size with diameters ranging from about 0.02 microm to greater than about 10 microm. A variety of agents can be encapsulated in liposomes. Hydrophobic agents partition in the bilayers and hydrophilic agents partition within the inner aqueous space(s). See e.g., Machy et al., Liposomes in Cell Biology and Pharmacology (John Libbey, 1987), and Ostro et al., American J. Hosp. Pharm. 46: 1576 (1989).

Liposomes can adsorb to virtually any type of cell and then release an incorporated agent. Alternatively, the liposome can fuse with the target cell, whereby the contents of the liposome empty into the target cell. Alternatively, a liposome may be endocytosed by cells that are phagocytic. Endocytosis is followed by intralysosomal degradation of

liposomal lipids and release of the encapsulated agents. Scherphof et al., Ann. N.Y. Acad. Sci., 446: 368 (1985).

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. See Colloidal Drug Delivery Systems, vol. 66 (J. Kreuter, ed., Marcel Dekker, Inc., 1994).

A “liposomal formulation”is an in vitro-created lipid vesicles in which an antigen of the present invention can be incorporated. Thus, “liposomally-bound” refers to a antigen that is partially incorporated or attached to a liposome. The immunogen of the present invention may be a liposomally-bound antigen which, but for said liposome, would not be an immunogen, or it may be immunogenic even in a liposome-free state.

Several different immunogens may be incorporated into the same liposome, or each into a different liposome and the liposomes administered together or separately to a subject.

A lipid-containing immunogen can be incorporated into a liposome because the lipid portion of the molecule will spontaneously integrate into the lipid bilayer. Thus, a glycolipopeptide may be presented on the “surface” of a liposome. Alternatively, a peptide may be encapsulated within a liposome. Techniques for preparing liposomes and formulating them with molecules such as peptides are well known to the skilled artisan.

Formation of a liposome requires one or more lipids. Any lipids may be used which, singly or in combination, can form a liposome bilayer structure. Usually, these lipids will include at least one phospholipid. The phospholipids may be phospholipids from natural sources, modified natural phospholipids, semisynthetic phospholipids, fully synthetic phospholipids, or phospholipids (necessarily synthetic) with normatural head groups. The phospholipids of greatest interest are phosphatidyl cholines, phosphatidyl phosphatidyl ethanolamines, phosphatidyl serines, phosphatidyl glycerols, phosphatidic acids, and phosphatidyl inositols.

The liposome may include neutral, positively charged, and/or negatively charged lipids. Phosphatidyl choline is a neutral phospholipid. Phosphatidyl glycerol is a negatively charged glycolipid. N-[1-(2,3-dioleylox)propyl]-N,N,N-trimethylammonium chloride is a positively charged synthetic lipid. Another is 3beta-[N—(N′,N″-dimethylaminoethane)-carbamoyl]-cholesterol.

Usually, the lipids will comprise one or more fatty acid groups. These may be saturated or unsaturated, and vary in carbon number, usually from 12-24 carbons. The phospholipids of particular interest are those with the following fatty acids: C12:0, C14:0, C16:0, C18:0, C18:1, C18:2, C18:3 (alpha and gamma), C20:0, C20:1, C20:3, C20:4, C20:5, C22:0, C22:5, C22:6, and C24:0, where the first number refers to the total number of carbons in the fatty acids chain, and the second to the number of double bonds. Fatty acids from mammalian or plant sources all have even numbers of carbon atoms, and their unsaturations are spaced at three carbon intervals, each with an intervening methylene group.

A liposome may include lipids with a special affinity for particular target cells. For example, lactosylceramide has a specific affinity for hepatocytes (and perhaps also for liver cancer cells).

In a preferred liposome formulation, the component lipids include phosphatidyl choline. More preferably they also include cholesterol, and still more preferably, also phosphatidyl glycerol. Cholesterol reduces the permeability of “fluid-crystalline state” bilayers.

Taking advantage of the self-assembling properties of lipids, one or more immunogens may be attached to the polar lipids that in turn become part of the liposome particle. Each immunogen comprises one or more antigenic determinants (epitopes). These epitopes may be B-cell epitopes (recognized by antibodies) or T-cell epitopes (recognized by T-cells). The liposome can act to adjuvant the immune response elicited by the associated immunogens. It is likely to be more effective than an adjuvant that is simply mixed with an immunogen, as it will have a higher local effective concentration.

Moreover, a hapten may be attached in place of the aforementioned immunogen. Like an immunogen, a hapten comprises an antigenic determinant, but by definition is too small to elicit an immune response on its own (typically, haptens are smaller than 5,000 daltons). In this case, the lipid moiety may act, not only as an adjuvant, but also as an immunogenic carrier, the conjugate of the hapten and the lipid acting as a synthetic immunogen (that is, a substance against which humoral and/or cellular immune responses may be elicited).

Even if the lipid does not act as an immunogenic carrier, the liposome borne hapten may still act as a synthetic antigen (that is, a substance which is recognized by a component of the humoral or cellular immune system, such as an antibody or T-cell). The term “antigen” includes both haptens and immunogens.

Adjuvants

It is generally understood that a synthetic antigen of low molecular weight can be weakly immunogenic, which is the biggest obstacle to the success of a fully synthetic vaccine. One way to improve the imunogenicity of such a synthetic antigen is to deliver it in the environment of an adjuvant. As conventionally known in the art, adjuvants are substances that act in conjunction with specific antigenic stimuli to enhance the specific response to the antigen. An ideal adjuvant is believed to non-specifically stimulate the immune system of the host, which upon the subsequent encounter of any foreign antigen can produce strong and specific immune response to that foreign antigen. Such strong and specific immune response, which is also characterized by its memory, can be produced only when T-lymphocytes (T-cells) of the host immune system are activated.

T-cell blastogenesis and IFN-g production as two important parameters for measuring the immune response. Experimentally, T-cell blastogenesis measures DNA synthesis that directly relates to T-cell proliferation, which in turn is the direct result of the T-cell activation. On the other hand, IFN-g is a major cytokine secreted by T-cells when they are activated. Therefore, both T-cell blastogenesis and IFN-g production indicate T-cell activation, which suggests the ability of an adjuvant in helping the host immune system to induce a strong and specific immune response to any protein-based antigen.

The compound is considered an adjuvant if it significantly (p=0.05) increases the level of either T-cell blastogenesis or of interferon gamma production in response to at least one liposome/immunogen combination relative to the level elicited by the immunogen alone. Preferably, it does both. Preferably, the increase is at least 10%, more preferably at least 50%, still more preferably, at least 100%.

A large number of adjuvants are known in the art, including Freund's complete adjuvant, saponin, DETOX (Ribi Immunochemicals), Montanide ISA-51, -50 and -70, QS-21, monophosphoryl lipid A and analogues thereof. A lipid adjuvant can be presented in the context of a liposome.

Several adjuvants are especially suitable for adjuvanting a liposome-delivered immunogen. Monophosphoryl lipid A (MPLA), for example, is an effective adjuvant that causes increased presentation of liposomal antigen to specific T Lymphocytes. Alving, C. R., Immunobiol., 187:430-446 (1993). The skilled artisan will recognize that lipid-based adjuvants, such as Lipid A and derivatives thereof, are also suitable. A muramyl dipeptide (MDP), when incorporated into liposomes, has also been shown to increase adjuvancity (Gupta R K et al., Adjuvants-A balance between toxicity and adjuvancity,” Vaccine, 11, 293-306 (1993)).

The oligonucleotides of the present invention are of course capable of serving as adjuvants, but other adjuvants may optionally be used.

### EXAMPLES

General: Melting points were not corrected. All air and moisture sensitive reactions were performed under nitrogen atmosphere. Anhydrous THF, DMF and dichloromethane were purchased from Aldrich and other dry solvents were prepared in the usual way. ACS grade solvents were purchased from Fisher and used for chromatography without distillation. TLC plates (silica gel 60 F₂₅₄, thickness 0.25 mm, Merck) and flash silica gel 60 (35-75 μm) for column chromatography were purchased from Rose Scientific, Canada. ¹H and ³¹P spectra were recorded either on a Brucker A M 300 MHz, Brucker A M 400 MHz, Varian Unity 500 MHz, or Brucker DRX 600 MHz spectrometers with TMS as internal standard for proton chemical shifts. Electron-spray mass spectrometric analyses were performed either on a MS50B or MSD1 SPC mass spectrometers.

### Example 1

### Preparation of Compound 8

To a solution of 1,2-dodecandiol (5.0 g, 24.7 mmol) and DIPEA (3.86 g, 5.2 mL, 27.7 mmol) in DCM/THF (10:1, 500 mL) was slowly added DMT-Cl solution (0.2 g, 27.2 mmol, in 100 mL DCM) over a period of 2 h at 0° C. The mixture was stirred at 0° C. for 3 h, followed by usual aqueous work-up. The product was purified by flash chromatography (hexane/ethyl acetate, 3:1) to give 8 (6.5 g, 52%). C₃₃H₄₄O₄ (504.44). TLC: R_{f}=0.67 (hexane/ethyl acetate, 2:1). ¹H NMR (300 MHz, CDCl₃): δ 0.88 (t, J=6.5 Hz, 3H), 1.24 (br s, 16H), 1.37 (m, 2H), 2.32 (d, J=3.5 Hz, 1H, OH), 3.00 (dd, _{J}=9.0, 7.0 Hz, 1H), 3.16 (dd, J=9.0, 3.5 Hz, 1H), 3.77 (m, 1H), 3.79 (s, 6H, 2 OCH₃), 6.85-7.60 (m, 13H, Ar—H).

### Example 2

### Preparation of Modified CPG Resin 9

Controlled pore glass (CPG) resin modified with long-chain-amino-alkyl group (lcaa-CPG) (0.6 g, 57 μmol) was treated with succinic andydride (57 mg, 0.57 mmol) in DMF (1.5 mL) overnight. Solvent was drained out and the resin washed with DMF (×5). The process was repeated once. The unreacted free amine on the resin was capped by treatment with NMI/Ac₂O/THF (1:1:8, v/v/v) for 5 min. The resin was then thoroughly washed with THF (×5) and CH₃CN (×5), dried under high vacuum to give the succinic acid-attached support.

The succinic acid-attached CPG resin (0.3 g, ˜28.5 mmol) was suspended in THF (2 mL) and 8 (43 mg, 85.5 μmol), DIC (14 μL), and DMAP (cat.) were added. The mixture was stirred at room temperature for 12 h. the solvent was drained out and the reaction was repeated once. The unreacted free carboxylic acid was capped by treating with methanol/DIC/DMAP for 3 h. The resin was then thoroughly washed with THF (×5) and DCM (×5), dried under vacuum to afford the lipid-modified CPG resin 9.

### Example 3

### Preparation of Compound 1-5

a) De-Tritylation

Lipid-modified CPG resin 9 (0.3 g, 28.5 μmol) was treated with trichloroacetic acid (TCA) (1% in DCM, w/v) for 5 min to remove the DMT-group. The process was repeated once for 1 min, followed by thorough wash with DCM (×5), acetonitrile (×5) and THF (×3).

b) Coupling

The resin was then swelled in dry THF (2 mL), and phosphoramidite reagent (42.8 μmol, 1.5 eq.) and tetrazole (3.0 mg, 42.8 mol) were added. The mixture was bubbled with N₂ gas for 16 h. After the removal of solvent and washing of the resin, the coupling with phosphoramidite was repeated once.

c) Capping

The unreacted free hydroxyl group on the resin was capped by treating with NMI/Ac₂O/THF (1:1:8, v/v/v) for 5 min. followed by thorough wash with DCM, acetonitrile and THF.

d) Oxidation

The resin was then treated with 2-butanone peroxide (, 2.0 mL, 0.1 M in DCM) for 5 min. and washed thoroughly with DCM, acetonitrile, and THF.

The synthesis was continued by repeating the cycle of De-tritylation, Coupling, Capping and Oxidation steps.

e) Cleavage and Final Deblocking

When the synthesis was complete, the resin was first treated with TCA (1% in DCM, w/v) for 5 min to remove the 5′-end DMT protection group, followed by thorough wash with DCM and acetonitrile. The resin was then treated with 20% ammonium hydroxide (aq.) at 50-55° C. for 24 h. The resin was filtered and the filtrate concentrated in vacuo, and the residue was re-dissolved in water-methanol (1:1) and purified by HPLC to give compound 1-5 (2.5-5.0 mg).

f) HPLC Conditions:

Column: Vydac C4 semi-Prep column, 10×250 mm; solvent A: 5% acetonitrile in water with 50 mM triethyl ammonium acetate (TEAA), pH=7.0; solvent B: 50% acetonitrile in water with 50 mM TEAA, pH=7.0. Gradient: from 20% acetronitrile to 45% acetonitrile in 30 min. flow rate: 3.0 mL/min. UV detection at e260 nm.

g) Structure Confirmation

The structures of compound 1-5 were confirmed by electron spray mass spectra data.

Compound 1: C₃₁H₅₀N8O₁₄P₂ (820.44), ES-MS (m/z, negative mode) found: 819 (M−H).

Compound 2: C₄₃H₇₅N₈O₂₃P₃ (1164.66), ES-MS (m/z, negative mode) found: 1163 (M−H).

Compound 3: C₇₃H₁₀₄N₂₂O₃₉P₆ (2098.86), ES-MS (m/z, negative mode) found: 2098 (M−H), 2121 (M+Na-2H).

Compound 4: C₇₁H₁₀₁N₁₉O₄₁P₆ (2061.83), ES-MS (m/z, negative mode) found: 2060 (M−H), 2083 (M+Na-2H), 2105 (M+2Na-3H), 2127 (M+3Nn−4H), 2171 (M+5Na-6H).

Compound 5: C₇₁H₁₀₀N₂₂O₃₈P₆ (2054.82), ES-MS (m/z, negative mode) found: 2054 (M−H), 2076 (M+Na-2H), 2098 (M+2Na-3H).

### Example 4

### Preparation of Compound 11

To a solution of 10 (5.0 g, 23.1 mmol) in dry DMF (200 ml) was added slowly NaH (2.2 g, 91.7 mmol) at 0° C. and the mixture was stirred for 1 h. 1-Bromohexadecane (17.8 g, 17.8 mL, 58.4 mmol) in DMF (30 mL) was added dropwise over a period of 1 h and the resulting mixture was stirred at room temperature for 16 h. water (5.0 ml) was added to quench the reaction and the DMF then removed under vacuum. Usual aqueous work-up gave a yellow syrup (crude 23.0 g). The syrup was treated with DCM/TFA/water (100:2.5:0.25, 200 mL) at room temperature for 3 h. The mixture was washed with water (50 mL×2) and sat. NaHCO₃ (50 mL). The organic layer was dried with Na₂SO₄, concentrated, and the residue was purified by flash chromatography (hexane/ethyl acetate, 4:1 then 2:1) to give 11 (9.73 g, 72% for two steps).

C₃₇H₇₆O4 (585.01). TLC: R_{f}=0.29 ((hexane/ethyl acetate, 3:1). ¹H NMR (300 MHz, CDCl₃): δ 0.88 (t, J=6.5 Hz, 6H, 2 CH₃), 1.25 (br s, 52H), 1.56 (m, 4H), 2.85 (t, J=5.0 Hz, 2H, 20H), 3.42 (t, J=6.5 Hz, 4H), 3.51 (s, 4H), 3.65 (d, J=5.0 Hz, 4H).

### Example 5

### Preparation of Compound 12

Compound 11 (2.33 g, 4.00 mmol) was dissolved in dry pyridine (30 mL) and DCM (27 mL), and DMAP (46 mg, 0.38 mmol) was added. DMT-CL (1.49 g, 4.40 mmol) was dissolved in DCM (13 mL) and added dropwise to the reaction flask. The reaction mixture was stirred at room temperature for 3 h. Methanol (5 ml) was added to quench the reaction and the solvent was removed by codistillation with toluene. The residue was re-dissolved in DCM (400 mL) and the solution was washed with sat. NaHCO₃ (150 mL). The organic layer was dried with Na₂SO₄, concentrated and the residue purified by flash chromatography (hexane/ethyl acetate, 10:1 then 5:1) to give 12 (3.05 g, 86%). C₁₈H₉₄O₆ (886.94). TLC: R_{f}=0.57 ((hexane/ethyl acetate, 5:1). ¹H NMR (500 MHz, CDCl₃): δ 0.86 (t, J=6.5 Hz, 6H, 2 CH₃), 1.23 (br s, 52 H), 1.48 (m, 4H), 2.95 (t, J=6.0 Hz, 1H, OH), 3.09 (s, 2H), 3.34 (m, 4H), 3.45 (d, J=9.0 Hz, 2H), 3.49 (d, J=9.0 Hz, 2H), 3.66 (d, J=6.0 Hz, 2H), 3.77 (s, 6H, 2 OCH₃), 6.78-7.41 (m, 13H, Ar—H).

### Example 6

### Preparation of Compound 13

Compound 12 (423 mg, 0.48 mmol) was dissolved in pyridine (7 mL) and succinic andydride (140 mg, 1.4 mmol) and DMAP (120 mg, 1.0 mmol) were added. The mixture was stirred at room temperature for 60 h and then concentrated by codistillation with toluene. The residue was purified by flash chromatography (hexane/ethyl acetate, 4:1:2%, with 0.1% pyridine) to give 13 (425 mg, 94%). R_{f}=0.19 (hexane/ethyl acetate, 5:1:2%). C₆₂H₉₈O₉ (986.72). ES-MS (m/z): found 1009.7 (M+Na). ¹H NMR (300 MHz, CDCl₃): δ 0.86 (t, J=6.5 Hz, 6H, 2 CH₃), 1.25 (br s, 52H), 1.48 (m, 4H), 2.46-2.59 (m, 4H), 3.09 (s, 2H), 3.32 (t, J=6.5 Hz, 4H), 3.36 (d, J=9.0 Hz, 2H), 3.42 (d, J=9.0 Hz, 2H), 3.80 (s, 6H, 2 OCH₃), 4.18 (s, 2H), 6.80-7.43 (m, 13H, Ar—H).

### Example 7

### Preparation of Compound 14

To a solution of 13 (0.18 g, 0.18 mmol) and p-nitrophenol (33.8 mg, 0.24 mmol) in DCM (10 mL) was added DCC (50 mg, 0.24 mmol). The mixture was stirred at room temperature for 6 h. Urea was filtered out and the filtrate concentrated. The residue was purified by flash chromatography (hexane/ethyl acetate, 4:1) to give 14 (0.18 g, 89%) as a clear syrup. C₆₈H₁₀₁NO₁₁ (1108.01). TLC: R_{f}=0.75 (hexane/ethyl acetate, 3:1). ¹H NMR (500 MHz, CDCl₃): δ 0.88 (t, J=6.5 Hz, 6H, 2 CH₃), 1.25 (br s, 52H), 1.47 (m, 4H), 2.59 (t, J=6.5 Hz, 2H), 3.10 (s, 2H), 3.32 (m, 4H), 3.37 (d, J=9.0 Hz, 2H), 3.41 (d, J=9.0 Hz, 2H), 3.78 (s, 6H), 2 OCH₃), 4.20 (s, 2H), 6.80-8.20 (m, 17H, Ar—H).

### Example 8

### Preparation of Modified CPG Resin 15

lcaa-CPG resin (1.0 g, 95 μmol) was suspended in dry DMF (3.0 mL) and 14 (0.26 g, 235 μmol) was added. DIPEA was added to adjust the pH to 9 and the mixture was bubbled with N₂ gas for three days. The solvent was drained out and the resin successively washed with DMF (×5) and DCM (×5). Unreacted free amine on the resin was capped with (NMI)/Ac₂O/THF (1:1:8, v/v/v) for 15 min. The resin was then thoroughly washed with acetonitrile (×5) and DC M (×5) and dried under vacuum to give lipid-modified CPG resin 15. The resin was tested with p-toluene sulfonic acid (0.1 M in acetonitrile) to give orange color, indicating the existence of DMT-group. Likewise, negative ninhydrin test result indicates the absence of the free amine on the resin.

### Example 9

### Preparation of Compound 18

Sodium hydride (817 mg, 34 mmol) was added to dry DMF (20 mL) and compound 17 (3.0 g, 23 mmol, dissolved in 20 mL of dry DMF) was added dropwise slowly at 0° C. The mixture was stirred at room temperature for 30 min and benzyl bromide (4.3 g, 2.97 mL, 25 mmol) was added dropwise slowly. The mixture was stirred at room temperature for 2 h. The solvent was then removed under high vacuo, followed by usual aqueous work-up. The crude product was purified by flash chromatography (hexane/ethyl acetate, 6:1) to give 18 (4.65 g, 94%). TLC: R_{f}=0.4⁶ (hexane/ethyl acetate, 6:1). C₁₃H₁₈O₃ (222.13). ES-MS (positive mode, m/z) found: 245 (M+Na). ¹H NMR (300 MHz, CDCl₃): δ 1.37 (s, 3H, CH₃), 1.42 (s, 3H, CH₃), 3.45 (dd, J=10.0, 5.5 Hz, 1H), 3.55 (dd, J=10.0, 5.5 Hz, 1H), 3.73 (dd, J=8.5, 6.5 Hz, 1H), 4.04 (dd, J=8.56.5 Hz, 1H), 4.30 (m, 1H), 4.53 (d, J=12.0 Hz, 1H), 4.59 (d, J=12.0 Hz, 1H), 7.35 (m, 5H, Ar—H).

### Example 10

### Preparation of Compound 19

Compound 18 (4.6 g, 20.7 mmol) was dissolved in HOAc—H₂O (4:1, 40 mL) and stirred at 40° C. for 1.5 h. the solvent was removed and the residue purified by flash chromatography (hexane/ethyl acetate/methanol, 1:1:0.1) to give 19 (3.6 g, 95%). TLC: R_{f}=0.36 (hexane/ethyl acetate/methanol, 1:2:0.1). C₁₀H₁₄O₃ (182.09). ¹H NMR (300 MHz, CDCl₃): δ 3.55 (dd, J=10.0, 6.5 Hz, 1H), 3.58 (dd, J=10.0, 4.0 Hz, 1H), 3.64 (dd, J=11.0, 5.5 Hz, 1H), 3.71 (dd, J=11.0, 3.5 Hz, 1H), 3.90 (m, 1H), 4.55 (s, 2H).

### Example 11

### Preparation of Compound 20

Compound 19 (5.4 g, 29.6 mmol) was dissolved in dry acetonitrile (700 ml) and triethyl amine (2.0 ml) was added. The mixture was cooled to −50° C. and benzoyl cyanide (3.5 g, 26.7 mmol, dissolved in 150 mL of dry acetonitrile) was added dropwise slowly under nitrogen atmosphere. The reaction mixture was further stirred at −50° C. for 1 h and the reaction was then quenched with methanol (20 mL). Solvent was then removed and the residue was purified by flash chromatography (hexane/ethyl acetate, 4:1) to give 20 (5.1 g, 60%) and the unreacted starting material 19 (1.0 g, 18%) was recovered. TLC: R_{f}=0.33 (hexane/ethyl acetate, 3:1). C₁₇H₁₈O₄ (286.12). ES-MS (positive mode, m/z) found: 287 (M+H), 309 (M+Na). ¹H NMR (300 MHz, CDCl₃): δ 2.65 (d, J=5.0 Hz, 1H, OH), 3.59 (dd, J=9.5, 6.0 Hz, 1H), 3.65 (dd, J=9.5, 5.0 Hz, 1H), 4.18 (m, 1H), 4.39 (dd, J=11.0, 5.5 Hz, 1H), 4.44 (dd, J=11.0, 4.5 Hz, 1H), 4.59 (s, 2H), 7.26-8.04 (m, 10H, Ar—H).

### Example 12

### Preparation of Compound 21

Compound 20 (1.8 g, 6.29 mmol) was dissolved in dry dichloroethane (20 mL) and paraformaldehyde (2.6 g) was added. The mixture was kept at 0° C. and HCl (g) was bubbled in for 3 h. HCl (g) was then removed and dry calcium chloride was added and the mixture stirred for 10 min. The solid was filtered and the washed with dry dichloroethane. The filtrate was concentrated and the residue dried briefly under high vacuo to afford crude 21 (2.0 g) which was used directly for the next step reaction.

### Example 13

### Preparation of Compound 23

Method A Compound 22 (480 mg, 2.23 mmol) was dissolved in dry DMF (10 mL) and sodium hydride (71 mg, 2.96 mmol) was added. The mixture was stirred at room temperature for 40 min and crude 21 (500 mg, ˜1.48 mmol, dissolved in 2 mL of DCM) was added dropwise slowly. The mixture was warmed to 60° C. and stirred for 20 h and then the reaction was quenched by adding methanol (1 mL). Solvent was removed, followed by usual aqueous work-up. From the organic layer, a viscous oily residue was obtained which was purified by flash chromatography (hexane/ethyl acetate/methanol, 1:1:0.01) to give 23 (230 mg, 30%).

Method B Compound 22 (480 mg, 2.23 mmol) was dissolved in dry DCM (12 mL) and Bis(trimethylsilyl)acetamide (BSA, 640 mg, 3.14 mmol) was added under nitrogen atmosphere. The mixture was stirred at room temperature for 45 min and the clear solution cooled to 0° C. Compound 21 (500 mg, ˜1.48 mmol, dissolved in 2 mL of DCM) and tetrabutyl ammonium iodide (8.0 mg, 0.02 mmol) were added and the mixture was stirred at room temperature for 16 h. The temperature was then brought to 60° C. and the mixture stirred for another 2 h. Dichloromethane (100 mL) was added and the organic layer was washed with saturated sodium bicarbonate solution (20 mL×3) and brine (20 mL), dried over sodium sulfate, and concentrated. The residue was purified by flash chromatography (hexane/ethyl acetate/methanol, 1:1:0.01) to give 23 (255 mg, 34%).

TLC: R_{f}=0.33 (hexane/ethyl acetate/methanol, 1:2:0.1). C₁₉H₂₇N₃O₆ (513.18). ES-MS (positive mode, m/z) found: 514 M+H), 536 (M+Na). ¹H NMR (600 MHz, CDCl₃): δ 3.63 (dd, J=10.5, 6.0 Hz, 1H), 4.34 (m, 1H), 4.42 (dd, J=12.0, 6.5 Hz, 1H), 4.46 (dd, J=12.0, 4.0 Hz, 1H), 4.53 (d, J=12.0 Hz, 1H), 4.57 (d, J=12.0 Hz, 1H), 5.44 (d, J=10.5 Hz, 1H), 5.51 (d, J=10.5 Hz, 1H), 7.26 (m, 1H), 7.34 (m, 4H), 7.41 (m, 2H), 7.49 (m, 1H), 7.53 (m, 2H), 7.63 (m, 1H), 7.76 (br s, 1H), 7.88 (br s, 2H), 7.96 (m, 1H), 7.98 (m, 1H), 8.55 (br s, 1H).

### Example 14

### Preparation of Compound 24

Compound 23 (800 mg, 1.56 mmol) was dissolved in dry DCM (65 mL) and boron trichloride (1 M solution in DCM, 2.5 mL) was added slowly at −78° C. under nitrogen atmosphere. The mixture was stirred at −78° C. for 1 h. The reaction was quenched by adding DCM-MeOH (1:1, 10 mL) and the solvent was removed. The residue was purified by flash chromatography (dichloromethane/methanol, 100:5) to give 24 (500 mg, 76%). TLC: R_{f}=0.58 (dichloromethane/methanol, 96:4). C₂₂H₂₁N₃O₆ (423.13). ES-MS (positive mode, m/z): 424.1 (M+H), 446.1 (M+Na). ¹H NMR (300 MHz, CDCl₃): δ 330 (br s, 1H, OH), 3.76 (dd, J=12.0, 6.0 Hz, 1H), 3.85 (dd, J=12.0, 4.0 Hz, 1H), 4.23 (m, 1H), 4.41 (dd, J=12.0, 6.0 Hz, 1H), 4.48 (dd, J=12.0, 4.5 Hz, 1H), 5.46 (d, J=11.0 Hz, 1H), 5.51 (d, J=11.0 Hz, 1H), 7.38-8.01 (m, 12H, Ar—H), 9.00 (br s, 1H).

### Example 15

### Preparation of Compound 25

Compound 24 (120 mg, 0.28 mmol) was dissolved in DCM (12 mL) and diisopropyl ethyl amine (DIPEA, 0.5 mL) was added. Phosphoramidite Cl—P(OCH₂CH₂CN)N(ⁱPr)₂ (93 mg, 91.3 μL, 0.39 mmol) was added under nitrogen atmosphere at room temperature. The mixture was stirred for 30 min and then diluted with DCM (50 mL). The DCM layer was washed with 10% NaHCO₃ solution (15 mL) and brine (15 mL), dried, and concentrated. The residue was purified by flash chromatography (hexane/ethyl acetate, 1:2, with 0.1% Et₃N) to give 25 (140 mg, 79%). TLC: R_{f}=0.54 (hexane/ethyl acetate, 1:2). C₃₁H₃₈N₅O₇P (623.25). ES-MS (positive) found 624.2 (M+H), 646.2 (M+Na). ¹H NMR (300 MHz, CDCl₃): δ 1.18 (s, 6H, 2 CH₃), 1.20 (s, 6H, 2 CH₃), 2.68 (t, J=6.0 Hz, 2H), 3.60 (m, 2H), 3.85 (m, 4H), 4.31 (m, 1H), 4.38-4.55 (m, 2H), 5.40 (dd, J=10.5, 1.5 Hz, 1H), 5.57 (dd, J=10.5, 8.0 Hz, 1H), 7.35-8.00 (m, 12H), 9.00 (br s, 1H). ³¹P NMR (500 MHz, CDCl₃): ä 150 27, 150.40.

### Example 16

### Preparation of Compound 26

Compound 17 (500 mg, 0.47 mL, 3.78 mmol), tert-butyl diphenylsilyl chloride (TBDPS—Cl, 1.25 g, 1.18 mL, 4.536 mmol) and imidazole (309 mg, 4.536 mmol) were dissolved in dry DMF (4 mL) and the mixture was stirred at room temperature for 3 h. The solvent was removed, followed by usual aqueous work-up. The product was purified by flash chromatography (hexane/ethyl acetate, 10:1) to give 26 (1.4 g, 100%). TLC: R_{f}=0.43 (hexane/ethyl acetate, 9:1). C₂₂H₃₀O₃Si (370.20). ES-MS (positive) found 393 (M+Na). ¹H NMR (300 MHz, CDCl₃): δ 1.05 (s, 9H, 3 CH₃), 1.35 (s, 3H, CH₃), 1.39 (s, 3H, CH₃), 3.65 (dd, J=10.5, 6.5 Hz, 1H), 3.74 (dd, J=10.5, 4.5 Hz, 1H), 3.92 (dd, J=8.5, 6.0 Hz, 1H), 4.07 (dd, J=8.5, 6.5 Hz, 1H), 4.20 (m, 1H), 7.35-7.68 (m, 10H).

### Example 17

### Preparation of Compound 27

Compound 26 (31.0 g, 83.78 mmol)<was treated with HOAc—H₂O (4:1, 100 mL) at 40° C. for 40 min. The solvent was removed and the residue purified by flash chromatography (hexane/ethyl acetate, 10:1 and then 4;1) to give 27 (23.8 g, 95%). TLC: R_{f}=0.40 (hexane/ethyl acetate, 1:1). C₁₉H₂₆O₃Si (330.20). ES-MS (positive) found 353 (M+Na). ¹H NMR (400 MHz, CDCl₃): δ 1.05 (s, 9H, 3 CH₃), 2.60 (br s, 2H, 2 OH), 3.64 (dd, J=11.5, 5.5 Hz, 1H), 3.69 (dd, J=11.5, 4.0 Hz, 1H), 3.70 (dd, J=10.0, 6.0 Hz, 1H), 3.74 (dd, J=10.0, 4.5 Hz, 1H), 3.81 (m, 1H), 7.37-7.67 (m, 10H, Ar—H).

### Example 18

### Preparation of Compound 28

To a solution of compound 27 (8.0 g, 24.2 mmol) and DIPEA (3.12 g, 4.2 mL, 24.2 mmol) in dry DCM (800 mL) was added dropwise slowly the solution of 4,4′-dimethoxytrityl chloride (DMT-Cl, dissolved in 200 mL of DCM) at room temperature under nitrogen atmosphere, and the mixture was stirred further for 2 h. The solvent was removed and the product purified by flash chromatography (DCM/ethyl acetate, 100:1, with 0.1% Et₃N) to give 28 (12.3 g, 80%). TLC: R_{f}=0.35 (DCM/ethyl acetate, 100:2). C₁₀H₄₄O₅Si (632.24). ES-MS (positive) found 655 (M+Na). ¹H NMR (300 MHz, CDCl₃): δ 0.99 (s, 9H, 3 CH₃), 2.44 (d, J=5.0 Hz, 1H, OH), 3.77 (m, 2H), 3.78 (s, 6H, 2 OCH₃), 3.88 (m, 1H), 6.80-7.85 (m, 23H, Ar—H).

### Example 19

### Preparation of Compound 29

Compound 28 (3.20 g, 5.06 mmol) was dissolved in dry THF (100 mL) and DIPEA (15 mL) was added. Methoxymethyl chloride (MOM-Cl, 4.89 g, 4.6 mL, 60.76 mmol) was added dropwise slowly at 0° C. and then the reaction mixture was stirred at 55° C. for 5 h. The mixture was cooled to 0° C. and sat NaHCO₃ (aq.) (20 mL) was added. The organic layer was separated and the aqueous layer extracted with ethyl acetate (3×40 mL). The combined organic layer was dried over Na₂SO4 and concentrated. The residue was purified by flash chromatography (hexane/ethyl acetate, 10:1, with 0.1% Et₃N) to give 29 (2.7 g, 79%). TLC: R_{f}=0.57 (hexane/ethyl acetate, 4:1). C₄₂H₄₈O₆Si (676.20). ES-MS (positive mode, m/z): found 699 (M+Na). ¹H NMR (300 MHz, CDCl₃): δ 1.00 (s, 9H, 3 CH₃), 3.21 (dd, J=10.0, 6.0 Hz, 1H), 3.27 (dd, J=10.0, 4.5 Hz, 1H), 3.31 (s, 3H, OCH₃), 3.74 (d, J=5.5 Hz, 2H). 3.77 (s, 6H, 2 OCH₃), 3.91 (m, 1H), 4.72 (d, J=11.5 Hz, 1H), 4.75 (d, J=11.5 Hz, 1H), 6.70-7.60 (m, 23H, Ar—H).

### Example 20

### Preparation of Compound 31

Compound 29 (500 mg, 0.74 mmol) was dissolved in dry DCM (10 mL) and DIPEA (2.0 mL) was added. Dimethylboron bromide solution (1.5 M in DCM, 2.5 mL, 3.75 mmol) was added at −78° C. and stirred for 1 h. The reaction was slowly warmed to room temperature and sodium iodide was added. The mixture was stirred at room temperature for 16 h. Meanwhile, compound 30 (246 mg, 1.11 mmol) was dissolved in dry DMF (6 mL) and sodium hydride (40 mg, 1.67 mmol) was added. The mixture was stirred at room temperature for 10 min and added slowly to the above reaction flask. The reaction mixture was stirred at room temperature for 1 h, then at 60° C. for 16 h and finally at 80° C. for 6 h. The solvent was then removed and the residue purified by flash chromatography (ethyl acetate/hexane/methanol, 2:1.5:0.1) to give 31 (185 mg. 29%). TLC: R_{f}=0.57 (hexane/ethyl acetate/methanol, 5:10:1). C₅₀H₅₅N₅O₇Si (865.40). ES-MS (positive mode, m/z): found 866 (M+H). ¹H NMR (600 MHz, CDCl₃): δ 0.95 (s, 9H, 3 CH₃), 1.25 (d, J=7.0 Hz, 3H, CH₃), 1.26 (d, J=7.0 Hz, 3H, CH₃), 2.72 (m, 1H), 3.14 (dd, J=10.0, 6.5 Hz, 1H), 3.18 (dd, J=10.0, 4.0 Hz, 1H), 3.66 (d, J=5.5 Hz, 2H), 3.76 (s, 6H, 2 OCH₃), 4.05 (m, 1H), 5.84 (d, J=10.5 Hz, 1H), 5.87 (d, J=10.5 Hz, 1H), 6.76 (m, 4H, Ar—H), 7.15-7.58 (m, 19H, Ar—H), 7.89 (s, 1H).

### Example 21

### Preparation of Compound 32

Compound 31 (220 mg, 0.254 mmol) was dissolved in dry THF (60 mL) and tetrabutyl ammonium fluoride solution (1.0 M in THF, 0.51 mL, 0.51 mmol) was added. The mixture was stirred at room temperature for 1 h and then the solvent was removed. The residue was purified by flash chromatography (hexane/ethyl acetate/methanol, 1:2:0.5) to give 32 (140 mg, 91%). TLC: R_{f}=0.36 (hexane/ethyl acetate/methanol, 1:2:0.5). C₃₄H₃₇N₅O, (627.28). ES-MS (positive mode, m/z) found: 650.3 (M+Na). ¹H NMR (600 MHz, CDCl₃): δ 1.16 (2 d, J=7.0 Hz, each 3H, 2 CH₃), 2.55 (m, 1H), 3.05 (dd, J=10.5, 4.0 Hz, 1H), 3.09 (dd, J=10.5, 6.0 Hz, 1H), 3.46 (dd, J=12.0, 7.0 Hz, 1H), 3.49 (dd, J=12.0, 3.5 Hz, 1H), 3.69 (s, 6H, 2 OCH₃), 3.79 (m, 1H), 5.75 (s, 2H), 6.70-7.27 (m, 13H), 7.85 (s, 1H).

### Example 22

### Preparation of Compound 33

1-Tetradecanol (200 mg 0.933 mmol) and DIPEA (0.5 mL) were dissolved in dry DCM (10 mL) and phosphoramidite reagent Cl—P(OCH₂CH₂CN)N(ⁱPr)₂ (265 mg, 260 μL, 1.1 mmol) was added. The mixture was stirred at room temperature for 1 h and then diluted with DCM (50 mL). The DCM layer was washed with 10% NaHCO₃ (aq.) (10 mL) and brine (10 mL), dried over Na₂SO₄, and concentrated. The residue was purified by flash chromatography (hexane/ethyl acetate, 1:4, with 1% Et₃N) to give 33 (382 mg, 100%) which was used directly in the next step reaction.

### Example 23

### Preparation of Compound 34

The mixture of 32 (50 mg, 0.080 mmol), 33 (70 mg, 0.160 mmol) and tetrazole (20 mg) in dry DCM (6 mL) was stirred at room temperature for 1 h. Then 2-butanone peroxide solution (1 M in DCM, 1.0 mL) was added and the reaction mixture was stirred at room temperature for 10 min. the solvent was removed and the residue purified by flash chromatography (hexane/ethyl acetate/methanol, 1:2:0.5) to give 34 (53 mg, 79%). TLC: R_{f}=0.22 (hexane/ethyl acetate/methanol, 1:2:0.2). C₅₁H₆₉N₆O₁₀P (956.48). ES-MS (positive mode, m/z) found: 957 (M+H), 979 (M+Na) ¹H NMR (500 MHz, CDCl₃): δ 0.88 (t, J=7.0 Hz, 3H, CH₃) 1.25 (m, 28H, 11 CH₂, 2 CH₃), 1.64 (m, 2H), 2.75 (m, 2H), 2.88 (m, 1H), 3.20 (m, 2H), 3.80 (s, 6H, 2 OCH₃), 4.00 (m, 2H), 4.05-4.24 (m, 5H). 5.85 (m, 2H), 6.80 (m, 4H), 7.20 (m, 9H), 8.00 (s, 1H), 9.25 (s, 1H), 12.20 (s, 1H).

### Example 24

### Preparation of Compound 35

Compound 34 (150 mg, 0.157 mmol) was treated with trichloroacetic acid solution (3% in DCM, w/v, 3 mL) at room temperature for 10 min. The mixture was concentrated in vacuo and the residue purified by flash chromatography (ethyl acetate/DCM/methanol, 2:8:0.5) to give 35 (101 mg, 99%). TLC: R_{f}=0.31 (ethyl acetate/DCM/methanol, 2:1:0.3). C₃₀H₅₁N₆O₈P (654.34). ES-MS (positive mode, m/z) found): 655.4 (M+H), 677.4 (M+Na).

### Example 25

### Preparation of Compound 36

The mixture of 25 (140 mg, 0.225 mmol), 35 (100 mg, 0.153 mmol) and tetrazole (20 mg) in dry DCM (7 mL) was stirred at room temperature for 3 h. 2-Butanone peroxide solution (1.0 M in DCM, 2.0 mL, 2.0 mmol) was added and the mixture was stirred for 10 min. The solution was then diluted with DCM (100 mL) and the organic layer washed successively with 10% NaHCO₃ (aq.) (20 mL) and brine (20 mL), dried over sodium sulfate and concentrated. The residue was purified by flash chromatography (ethyl acetate/hexane/DCM/methanol, 5:2:2:1) to give 36 (117 mg, 64%). TLC: R_{f}=0.34 (DCM/methanol, 9:1). C₅₅H₇₄N₁₀O₁₆P₂ (1192.47). ES-MS (positive mode, m/z) found: 1193.5 (M+H), 1215.5 (M+Na). ¹H NMR (600 MHz, CDCl₃): δ 0.86 (t, J=7.5 Hz, 3H, CH₃) 1.25 (m, 28H, 11 CH₂, 2 CH₃), 1.65 (m, 2H), 2.75 (m, 5H), 3.30 (m, 2H), 4.10-4.60 (m, 14H), 5.44 (m, 1H), 5.58 (m, 1H), 5.82 (m, 2H), 5.88 (m, 2H), 7.38-8.20 (m, 13H), 9.40 (br s, 1H, NH), 12.20 (br s, 1H, NH).

### Example 26

### Preparation of Compound 6

Compound 36 was dissolved in ethanol (2.0 mL) and conc. ammonium hydroxide (58%, 4.0 mL) was added. The mixture was stirred at 55° C. for 24 h and then concentrated in vacuo. The residue was re-dissolved in water (8.0 mL) and filtered through 0.22 μm filter and the clear solution was lyophilized to give the crude product (80 mg) which was further purified by HPLC to give 6 (41.3 mg, 61%). (HPLC condition was given in Example 3, as that described for the purification of compound 1-5). TLC: R_{f}=0.16 (chloroform/methanol/water/conc. ammonium hydroxide, 5:3:0.5:0.5). C₃₁H₅₄N₈O₁₃P₂ (808.33). ES-MS (positive mode, m/z) found: 831 (M+Na), 853 (M+2Na—H), 875 (M+3Na-2H), 897 (M+4Na-3H). ¹H NMR (500 MHz, DMSO-d₆): δ 0.85 (t, J=7.0 Hz, 3H, CH₃), 1.22 (br s, 22H, 11 CH₂), 1.41 (m, 2H), 3.39 (m, 2H), 3.52-3.72 (m, 9H), 3.94 (m, 1H), 5.12 (d, J=10.0 Hz, 1H), 5.16 (d, J=10.0 Hz, 1H), 5.68 (s, 2H), 5.75 (d, J=7.5 Hz, 1H), 6.25 (br s, 2H), 7.12 (s, 1H), 7.48 (s, 1H), 7.68 (d, J=7.5 Hz, 1H), 8.11 (s, 1H).

### Example 27

### Preparation of Compound 7

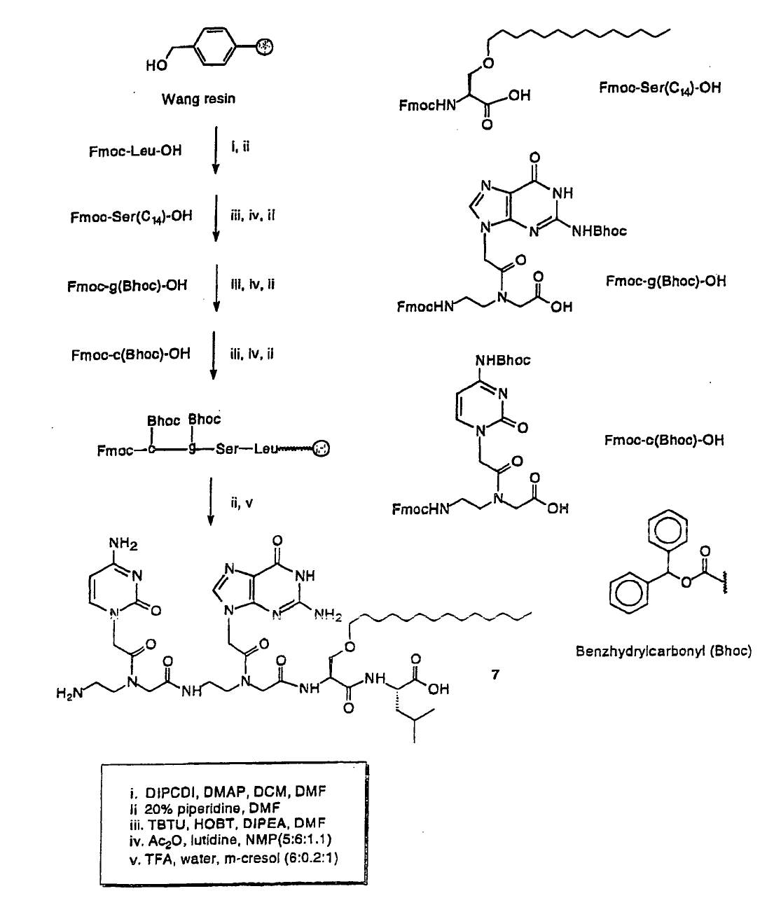

The hybrid structure of peptide and peptide nucleic acid was prepared using peptide synthesizer by employing the technique of standard solid phase peptide synthesis. Wang resin was chosen as the solid support using Fmoc/Bhoc chemistry. The reaction scheme and reaction conditions are described in FIG. 15. after the cleavage from the resin, the product was purified by HPLC and the structure confirmed by ES-MS spectroscopic data.

Compound 7: C₄₄H₇₂N₁₄O₁₀ (956.55). ES-MS (positive mode, m/z) found: 957.5 (M+H), 979.5 (M+Na), 1001.5 (M+2Na—H), 1023.5 (M+3Nz-2H), 1045.5 (M+4Nz-3H).

### Example 28

### Preparation of BLP25 Liposomal Vaccine

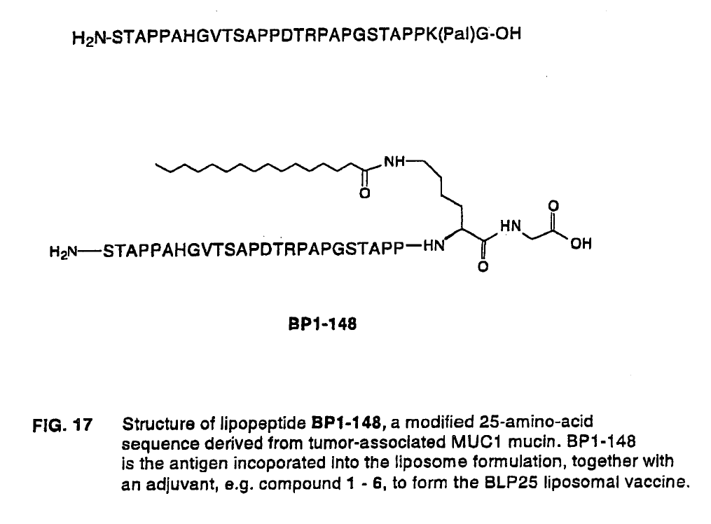

Typically, the liposomal formulation was composed of 400 μg of MUC1-based lipopeptide BP1-148 (FIG. 17), H₂N-STAPPAHGVTSAPDTRPAPGSTAPPK(Pal)G-OH, 200 μg of CpG analog 1-6, 6.94 mg of cholesterol, 1.46 mg of dimyristoyl phosphatidylglycerol (DMPC) and 11.62 mg of dipalmitoyl phosphatidylcholine (DPPC) per 1 ml of saline (0.9% NaCl solution).

The liposomal constructs were formulated by first dissolving the phospholipids, cholesterol and CpG analog 1-6 in tert-butanol at about 53° C. Lipopeptide and water (5%, v/v) were then added to the tert-butanol solution. The resulting clear 95% tert-butanol solution was injected into about 4 volumes of rapidly stirred water at about 50° C., using a glass syringe with an 18-gauge needle. The small unilamellar vesicles (SUV) formed in this process were cooled, sterilized by filtration through a 0.22 μm membrane filter, filled into vials and lyophilized. The dry powder was re-hydrated with sterile saline before injection, resulting in the formation of multilamellar large vesicles (MLV). The liposomes formed are used to immunize mice.

### Example 29

### Mice Immunized with Liposomal Vaccines

Groups of C57-Black mice were immunized subcutaneously with the BLP25 liposomal vaccine containing 400 μg of MUC1-based lipopeptide BP1-148 (FIG. 17), and 200 μg of CpG analog per dose. Nine days after vaccine injection mice were sacrificed and lymphocytes were taken from the draining lymph nodes (local response) or from the spleens (systemic response) to determine the immune response in each group. The lymphocytes taken from immunized mice were incubated in in vitro cultures in the presence of MUC1-based boosting antigen BP1-151, which has the peptide sequence H₂N-STAPPAHGVTSAPDTRPAPGSTAPPK-OH (SEQ ID NO:11) This sequence corresponds to SEQ ID NO:2 plus a terminal lysine.

### Example 30

### Measurement of T-Cell Proliferation

T-cell proliferation is evaluated using a standard ³H thymidine incorporation assay. Briefly, nylon wool passed inguinal lymph node lymphocytes, at 0.25×10⁶/well, pooled from each mouse group, are added to a culture containing naive mitomycin C-treated syngeneic splenocytes at 0.25×10⁶/well, which serve as antigen presenting cells (APCs). To each well 20 μg of MUC1-based 25-mer peptide is added as boosting antigen. The culture is incubated for 72 h in a total volume of 300 μL/well, followed by the addition of 1 μCi of ³H-thymidine in a volume of 50 μL. The plates are incubated for an additional 18-20 h. Cells are harvested and [³H]dTh incorporation is measured by liquid scintillation counter. T-cell proliferation results corresponding to various liposomal vaccines adjuvanted with 1-6 or the reference natural R595 lipid A are shown in FIG. 16.

R595 lipid A is the natural detoxified lipid A product isolated from the bacteria Salmonella minnesota, R595. This material is commercially available from Avanti Polar Lipids, Inc., USA. R595 lipid A is a strong vaccine adjuvant currently under clinical investigation for human use, and therefore it is chosen here as a reference to compare the immune stimulatory (adjuvant) properties of compound 1-6.

As shown in FIG. 16, compound 1-6 demonstrate strong immunoadjuvant activity in enhancing antigen specific T cell proliferation. All these CpG analogues show the same or higher magnitude of activity compared to R595 lipid A. Compound 3-6 demonstrate obviously stronger activity then R595 lipid A. Interestingly, the glycerol-based CpG analogue 6 has stronger activity than compound 1 and 2 which are based on natural DNA backbone. Collectively, these data show that short oligonucleotide sequence (as small as dinucleotide) and their structural mimics containing unmethylated CpG unit, when modified with strong lipophilic group(s), has strong immune stimulatory properties.

The biological activity of compound 7 has not been evaluated.

### REFERENCES

Akira, S., Takeda, K. & Kaisho, T. Nature Immunol. 2001, 2, 675-680.
Alexopoulou, L., Czopik-Holt, A, Medzhitov, R. & Flavell, R. Nature, 2001, 413, 732-738.
Hemmi, H. et al, Nataure, 2000, 408, 740-745.
Janeway, C. W. Jr. Immunol. Today, 1992, 13, 11-16.
Krieg, A. M. et al, Nature, 1995, 374, 546-549.
Krutzik, S. R., Sieling, R. A. & Modlin, R. L., Curr. Opin. Immunol. 2001, 13, 104-108.
Medzhitov, R., Nature Rev. Immunol. 2001, 1, 135-145.
Takeuchi, O. et al, Immunity, 1999, 11, 443-451.
Uhlmann, E. & Peyman, A. Chem. Rev. 1990, 90, 543-586.
Zamecnik, P. C. & Stephenson, M. L. Proc. Natl. Acad. Sci. U.S.A. 1978, 75. 280-284.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

The invention, as contemplated by applicant(s), includes but is not limited to the subject matter set forth in the appended claims, and presently unclaimed combinations thereof. It further includes such subject matter further limited, if not already such, to that which overcomes one or more of the disclosed deficiencies in the prior art. To the extent that any claims encroach on subject matter disclosed or suggested by the prior art, applicant(s) contemplate the invention(s) corresponding to such claims with the encroaching subject matter excised.

All references, including patents, patent applications, books, articles, and online sources, cited anywhere in this specification are hereby incorporated by reference, as are any references cited by said references.

1. A method of stimulating the immune system of a subject which comprises administering to the subject an immunologically effective amount of an immunostimulatory molecule which comprises at least one oligonucleotide strand which comprises
(1) at least one nucleotide sequence comprising a plurality of nucleotides, each nucleotide comprising a nucleobase, and thereby also comprising at least one CxG dinucleotide unit or analogue thereof, and
(2) at least one covalently incorporated lipophilic group.

2. The method of claim 1 in which element (1) comprises a CxG dinucleotide unit.

3. The method of claim 1 in which the CxG dinucleotide unit is a CpG dinucleotide unit.

4. The method of claim 1 in which at least one lipophilic group is a strongly lipophilic group.

5. The method of claim 1 in which at least one lipophilic group is a highly lipophilic (Meylan) group.

6. The method of claim 1 in which at least one lipophilic group has a predicted logP, according to the Meylan algorithm, of at least 4.

7. The method of claim 1 in which at least one lipophilic group has a predicted logP, according to the Meylan algorithm, of at least 7.

8. The method of claim 1 in which at least one lipophilic group has a predicted logP, according to the Meylan algorithm, of at least 10.

9. The method of claim 1 in which at least one lipophilic group is selected from the group consisting of
—(CH₂)₄NH·CO—(CH₂)ₙCH₃ (a)
where n=an integer with values ranging from 6 to 26, 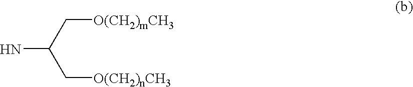
where m and n are independent integers with values ranging from 6 to 26,
(c)
—XR wherein X is —O—, —S—, or —NH— and —R is aliphatic, and
(d)
—XR wherein X is —O—, —S—, or —NH— and —R is at least partially aromatic.

10. The method of claim 9 where —R is a group selected from the group consisting of one of the following structures: 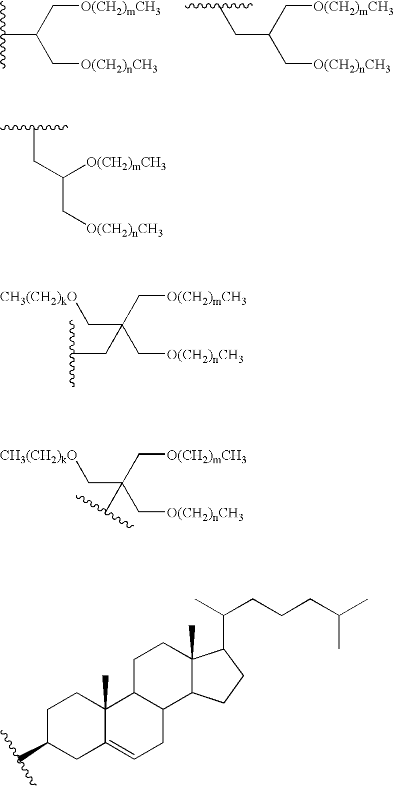
where m, n, and k are independent integers with values ranging from 3 to 30.

11. The method of claim 1 in which at least one lipophilic group is one of the lipophilic groups depicted in FIG. 2.

12. The method of claim 1 where said molecule comprises at least two lipophilic groups.

13. The method of claim 12 where said molecule comprises at least two strongly lipophilic groups.

14. The method of claim 12 where said molecule comprises at least two highly lipophilic (Meylan) groups.

15. The method of claim 1 in which there are fewer than 8 nucleobases on each nucleotide strand.

16. The method of claim 15 in which there are fewer than 5 nucleobases on each nucleotide strand.

17. The method of claim 1 in which each of the nucleobases is selected from the group consisting of adenine, guanine, thymine, cytosine, uracil, and hypoxanthine.

18. The method of claim 1 in which each of the nucleobases is selected from the group consisting of adenine, guanine, thymine, and cytosine.

19. The method of claim 1 wherein at least one nucleotide comprises a nucleobase-carbohydrate nucleoside.

20. The method of claim 19 in which the carbohydrate is a monosaccharide.

21. The method of claim 19 in which the number of carbon atoms in the monosaccharide is 3-8.

22. The method of claim 21 in which the monosaccharide is a pentose.

23. The method of claim 20 in which the monosaccharide is an aldose.

24. The method of claim 20 in which the monosaccharide is cyclized.

25. The method of claim 19 in which the monosaccharide is a pyranose.

26. The method of claim 20 in which the monosaccharide is a ribose or a 2-deoxyribose.

27. The method of claim 1 in which all of the nucleotides of one strand comprise a nucleobase-carbohydrate nucleoside.

28. The method of claim 1 in which at least one nucleotide further comprises a phosphate.

29. The method of claim 28 in which at least one nucleotide comprises one and only one phosphate.

30. The method of claim 29 in which at least one strand is DNA or RNA.

31. The method of claim 1 in which at least one lipophilic group is covalently incorporated into a free end of at least one strand.

32. The method of claim 1 in which at least one lipophilic group is covalently incorporated into the 3′ end of at least one strand.

33. The method of claim 31 in which the lipophilic group is attached to the end through a phosphate group.

34. The method of claim 1 in which at least one lipophilic group is incorporated into an internucleoside linkage.

35. The method of claim 1 in which at least one lipophilic group is a substituent of a nucleobase.

36. The method of claim 1 in which at least part of the oligonucleotide has a backbone which differs from that of DNA and RNA.

37. The method of claim 36 in which the backbone differs in that the internucleoside linkage is not a phosphate group.

38. The method of claim 36 in which the backbone differs in that at least one nucleotide is a non-normal nucleotide which does not comprise ribose or 2-deoxyribose.

39. The method of claim 38 in which the non-normal nucleoside comprises a sugar.

40. The method of claim 38 in which the non-normal nucleotide does not comprise a sugar.

41. The method of claim 40 in which the oligonucleotide is at least partially a PNA oligomer.

42. The method of claim 40 in which at least one non-normal nucleotide comprises a non-normal nucleoside of the form
nucleobase-O-alkyl
where the —O-alkyl is the residue of a polyol, and the alkyl is not more than 6 carbon atoms.

43. The method of claim 42 wherein the polyol is glycerol, and hence the alkyl is 3 carbon atoms.

44. The method of claim 36 in which at least non-normal nucleoside is bound to a phosphate group.

45. The method of claim 44 in which there are two adjacent such non-normal nucleosides and the internucleoside linkage between them is a phosphate group.

46. The method of claim 44 in which there are two adjacent such non-normal nucleosides and the internucleoside linkage between them is
-phosphate group-linker Z-phosphate group-,
where linker Z is aliphatic.

47. The method of claim 46 in which linker Z is of the form -[small alkyl-O]ₙ, where n is 1 to 20, and small alkyl is not more than 6 carbon.

48. The method of claim 47 in which linker Z is —[CH₂CH₂O]ₙ—.

49. The method of claim 1 in which the dinucleotide unit comprises a non-natural nucleoside, or an internucleoside linkage which is not a phosphate group.

50. The method of claim 1 in which the CxG dinucleotide unit comprises two non-natural nucleosides and the internucleoside linkage between them is a phosphate group.

51. The method of claim 49 in which the dinucleotide unit comprises two non-natural nucleosides and the internucleoside linkage between them is
-phosphate group-linker Z-phosphate group-,
where linker Z is aliphatic.

52. The method of claim 51 in which linker Z is —[CH₂CH₂O]ₙ— and n is 1 to 20.

53. The method of claim 49 in which the dinucleotide unit is a PNA oligomer.

54. The method of claim 49 in which the dinucleotide unit is a GNA oligomer.

55. The method of claim 1 in which the molecule lacks double stranded structure.

56. The method of claim 1 in which the molecule has at least some double stranded structure.

57. The method of claim 1 in which there are no more than seven nucleobases in each oligonucleotide strand.

58. The method of claim 1 in which there are no more than four nucleobases in each oligonucleotide strand.

59. The method of claim 1 in which the molecule further comprises at least one epitope.

60. The method of claim 59 wherein at least one epitope is a carbohydrate epitope.

61. The method of claim 59 wherein at least one epitope is a peptide epitope.

62. The method of claim 59 wherein at least one epitope is a B-cell epitope.

63. The method of claim 59 wherein at least one epitope is a T-cell epitope.

64. The method of claim 59 wherein at least one epitope is a MUC1 epitope.

65. The method of claim 1 in which the oligonucleotide is cyclized, so as to lack a free end, and the lipophilic groups are incorporated elsewhere in the molecule.

66. The method of claim 1 where said molecule which comprises two or more segments, each segment consisting of nucleosides joined to each other by short internucleoside linkages, each segment being joined to at least one other segment by a long internucleoside linkage,
at least two of said segments each comprising at least one CxG dinucleotide unit or analogue thereof.

67. The method of claim 66 in which said segments are connected by said internucleoside linkages to form one or more linear chains.

68. The method of claim 66 in which two or more segments are cyclized by two or more internucleoside linkages.

69. The molecule of claim 66 in which at least one internucleoside linkage is a higher order linkage which simultaneously links three or more segments to each other, whereby said segments are connected to form a branched structure.

70. The molecule of claim 66 in which at least one internucleoside linkage comprises a pentaerythritol unit.

71. The molecule of claim 66 in which the molecule has the following structural feature 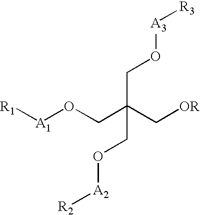
wherein R is a lipophilic group; A₁, A₂ and A₃ are independently a linking arm of 0 to 20 atoms length; R₁, R₂ and R₃ are independently chosen from the group consisting of a hydrogen atom, a lipophilic group or an oligo-nucleotide residue.

72. The method of claim 1 where said nucleotide sequence comprises at least one pair of adjacent thymine nucleobases which are dimerized to form a thymine dimer.

73. The method of claim 1 in which at least one lipophilic group is covalently incorporated into the 5′ end of an oligonucleotide strand.

74. The method of claim 1 in which the molecule does not have cytotoxic activity against cancer cells.

75. The method of claim 1 in which the subject is not suffering from a cancer.

76. The method of claim 1 in which the subject is not being medicated with any otehr cancer preventative.

77. The method of claim 1 in which the molecule potentiates the specific innate immune response to a pathogen or cancer already present in the subject.

78. The method of claim 1 which further comprises administering a pharmaceutical composition comprising an immunogen to the subject, said molecule potentiating the specific elicited immune response to said immunogen.

79. The method of claim 78 in which the molecule and the immunogen are administered simultaneously.

80. The method of claim 79 in which the molecule and the immunogen are administered in the same composition.

81. The method of claim 78 in which the immunostimulatory oligonucleotide molecule is also an immunogen which elicits a specific immune response protective against said pathogen or cancer.

82. (canceled)

83. An immunostimulatory molecule which comprises at least one oligonucleotide strand, said strand comprising a nucleotide sequence, the nucleotide sequence comprising a plurality of nucleotides, each nucleotide comprising a nucleobase, and thereby also comprising at least one CxG dinucleotide unit or analogue thereof, and
(2) at least one covalently incorporated lipophilic group,
where the number of nucleotides in said strand is less than eight.

84. The molecule of claim 83 which comprises at least one oligonucleotide strand, said strand comprising a nucleotide sequence, the nucleotide sequence comprising a plurality of nucleotides, each nucleotide comprising a nucleobase, and thereby also comprising at least one CxG dinucleotide unit, and
(2) at least one covalently incorporated lipophilic group, where the number of nucleotides in said strand is less than eight.

85. An immunostimulatory molecule which comprises (I) at least one oligonucleotide strand which comprises
(1) at least one nucleotide sequence comprising a plurality of nucleotides, each nucleotide comprising a nucleobase, and thereby also comprising at least one CxG dinucleotide unit or analogue thereof, and
(2) at least one covalently incorporated lipophilic group, and
(II) an epitope.

86. The molecule of claim 85 wherein at least one epitope is a carbohydrate epitope.

87. The molecule of claim 85 wherein at least one epitope is a peptide epitope.

88. The molecule of claim 85 wherein at least one epitope is a B-cell epitope.

89. The molecule of claim 85 wherein at least one epitope is a T-cell epitope.

90. The molecule of claim 85 wherein at least one epitope is a MUC1 epitope.

91. (canceled)

92. An immunostimulatory molecule which comprises (I) at least one oligonucleotide strand which comprises
(1) at least one nucleotide sequence comprising a plurality of nucleotides, each nucleotide comprising a nucleoside, each nucleoside comprising a nucleobase, each nucleoside being joined to at least one other nucleoside by an internucleoside linkage,
said molecule comprising two or more segments, each segment consisting of nucleosides joined to each other by short internucleoside linkages, each segment being joined to at least one other segment by a long internucleoside linkage,
at least two of said segments each comprising at least one CxG dinucleotide unit or analogue thereof, and
(2) at least one covalently incorporated lipophilic group.

93. The molecule of claim 92 in which said segments are connected by said internucleoside linkages to form one or more linear chains.

94. The molecule of claim 92 in which one or more segments are connected by one or more of said long internucleoside linkages to form a cyclic structure.

95. The molecule of claim 92 in which at least one internucleoside linkage is a higher order linkage which simultaneously links three or more segments to each other, whereby said segments are connected to form a branched structure.

96. The molecule of claim 92 in which at least one internucleoside linkage comprises a pentaerythritol unit

97. The molecule of claim 92 in which the molecule has the following structural feature 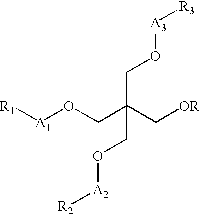
wherein R is a lipophilic group; A₁, A₂ and A₃ are independently a linking arm of 0 to 20 atoms length; R₁, R₂ and R₃ are independently chosen from the group consisting of a hydrogen atom, a lipophilic group or an oligo-nucleotide residue.

98. The molecule of claim 92 which comprises (I) at least one oligonucleotide strand which comprises
(1) at least one nucleotide sequence comprising a plurality of nucleotides, each nucleotide comprising a nucleoside, each nucleoside comprising a nucleobase, each nucleoside being joined to at least one other nucleoside by an internucleoside linkage,
said molecule comprising two or more segments, each segment consisting of nucleosides joined to each other by short internucleoside linkages, each segment being joined to at least one other segment by a long internucleoside linkage,
at least two of said segments each comprising at least one CxG dinucleotide unit, and (2) at least one covalently incorporated lipophilic group.

99. An immunostimulatory molecule which comprises at least one oligonucleotide strand, said strand comprising
(1) a nucleotide sequence, the nucleotide sequence comprising a plurality of nucleotides, each nucleotide comprising a nucleobase, and thereby also comprising at least one CxG dinucleotide unit or analogue thereof, and
(2) at least one covalently incorporated lipophilic group,
where said nucleotide sequence comprises at least one pair of adjacent thymine nucleobases which are dimerized to form a thymine dimer.

100. The method of claim 1 where none of the internucleoside linkages is selected from the group consisting of poly(N-vinyl), poly (methacryloxyethyl), poly(methacrylamide), and poly(etheylenimine).


SEQUENCE LISTING (12 sequences)

SEQ ID NO: 1 - DNA, 10 bp, Artificial Sequence
  Synthesizable sequence, corresponds to 128H described in Cheng et al. USP 5,646,126
cacacgtgtg

SEQ ID NO: 2 - protein, 25 aa, Artificial Sequence
  Synthesized peptide shown in Fig. 17 BP1-148
STAPPAHGVTSAPDTRPAPGSTAPP

SEQ ID NO: 3 - DNA, 20 bp, Artificial Sequence
  Synthesizable activating sequence, shown in Fig. 1
ggtgcatcgatgcagggggg

SEQ ID NO: 4 - protein, 10 aa, Leishmani major
EAEEAARLQA

SEQ ID NO: 5 - protein, 25 aa, Artificial Sequence
  MUC1 repeat consensus sequence
PAPGSTAPPAQTAHGVTSAPDETSR

SEQ ID NO: 6 - protein, 12 aa, Artificial Sequence
  MUC1 fragment
PGSTAPPAHGVT

SEQ ID NO: 7 - protein, 9 aa, Artificial Sequence
  MUC1 fragment
TLAPATEPA

SEQ ID NO: 8 - protein, 9 aa, Artificial Sequence
  MUC1 fragment
ALGSTAPPV

SEQ ID NO: 9 - protein, 9 aa, Artificial Sequence
  MUC1 fragment
FLSFHISNL

SEQ ID NO: 10 - protein, 20 aa, Artificial Sequence
  MUC1 repeat
GVTSAPDTRPAPGSTAPPAH

SEQ ID NO: 11 - protein, 28 aa, Artificial Sequence
  Synthesized peptide shown in Fig. 17 as intermediate
STAPPAHGVTSAPPDTRPAPGSTAPPKG

SEQ ID NO: 12 - protein, 12 aa, Plasmodium falciparum
NANPNANPNANP